(19) United States
(12) Patent Application Publication
(10) Pub. No.: US 2009/0247579 A1
(43) Pub. Date: Oct. 1, 2009

(54) 2-[3H-THIAZOL-2-YLIDINEMETHYL]PYRIDINES AND RELATED COMPOUNDS AND THEIR USE

(75) Inventors: Robert Brown, Strathclyde (GB); Jane Plumb, Strathclyde (GB); Gladys Wynne Aherne, Surrey (GB); Edward McDonald, Surrey (GB); Keith Jones, Surrey (GB); Stephen Hilton, Surrey (GB)

Correspondence Address: MICHAEL BEST & FRIEDRICH LLP, ONE SOUTH PINCKNEY STREET, P O BOX 1806, MADISON, WI, 53701, US

(73) Assignee: CANCER RESEARCH TECHNOLOGY LIMITED

(21) Appl. No.: 12/280,081

(22) PCT Filed: Feb. 21, 2007
(86) PCT No.: PCT/GB2007/000608

§ 371 Date: Aug. 20, 2008

(30) Foreign Application Priority Data
Feb. 21, 2006 (GB) 0603455.7

(51) Int. Cl. A61K 31/4709; A61K 31/4439; C07D 417/06

(52) U.S. Cl. 514/314; 435/375; 514/342; 546/176; 546/269.7

(57) ABSTRACT

The present invention pertains to certain 2-[3H-thiazol-2-ylidinemethyl]pyridine compounds and analogs thereof, which, inter alia, inhibit cell proliferation, treat cancer, etc., and more specifically to compounds of the following formula, wherein R^{A1}, R^{A2}, R^{A3}, R^{A4}, R^{B1}, R^{B2}, R^{NA}, R^{NB}, and X⁻ are as defined herein:

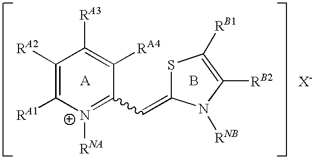

The present invention also pertains to pharmaceutical compositions comprising such compounds, and the use of such compounds and compositions, both in vitro and in vivo, to inhibit cell proliferation, and in the treatment of proliferative conditions such as cancer, etc.

### RELATED APPLICATION

This application is related to: United Kingdom patent application number 0603455.7 filed 21 Feb. 2006; the contents of which are incorporated herein by reference in their entirety.

### TECHNICAL FIELD

The present invention relates to therapeutic compounds, and more particularly, to certain 2-[3H-thiazol-2-ylidinemethyl]pyridine compounds and analogs thereof, which, inter alia, inhibit cell proliferation, treat cancer, etc. The present invention also pertains to pharmaceutical compositions comprising such compounds, and the use of such compounds and compositions, both in vitro and in vivo, to inhibit cell proliferation, and in the treatment of proliferative conditions such as cancer, etc.

### BACKGROUND

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiments.

Cancer is the uncontrolled growth of cells due to aberrations in growth control mechanisms. During cancer initiation and development, most tumours and leukaemia develop certain hallmarks such as evasion of apoptosis, insensitivity to anti-growth signals, limitless replicative potential, self-sufficiency in growth signals, sustained angiogenesis, and tissue invasion. Although many tumours can be treated successfully by surgical removal if detected early, once the tumour cells have metastasised and spread, then radio- or chemo-therapy are usually required, either to control symptoms or to improve patient survival. Some tumours and leukaemia are intrinsically resistant to chemotherapy and show limited response to treatment, however many are initially chemosensitive and will respond to chemotherapy. The vast majority of tumours or leukaemia that initially respond to chemotherapy will however eventually recur months or years following the end of chemotherapy. Tumours that recur can respond again to the initial treatment, but most eventually will fail to respond to chemotherapy following multiple treatment cycles. Such tumours have acquired resistance to chemotherapy and indeed can acquire resistance to therapies that have not yet been used to treat the tumour.

Mutations in DNA mismatch repair (MMR) genes occurs in humans in the cancer susceptibility syndrome hereditary non-polyposis colorectal carcinoma (HNPCC), which results in a predisposition towards colorectal carcinoma as well as a number of other tumors, including adenocarcinomas of the endometrium, stomach tumors and ovarian tumors (see, e.g., Lynch, 1993). The terms “MMR” and “DNA MMR” are used interchangeably herein. Defective MMR as measured by acquisition of microsatellite instability (MSI) has also been detected in a wide variety of sporadically occurring tumour types, including ones not associated with HNPCC (see, e.g., Eshleman et al., 1995). However, mutations of MMR genes have only been observed at low frequency in sporadic tumors with MSI and evidence suggests that methylation of the promoter of MMR genes leading to transcriptional silencing of MMR genes is an alternative to mutational inactivation of MMR genes in such sporadic tumours (see, e.g., Kane et al., 1997). Normal cells have functional MMR and do not show microsatellite instability (see, e.g., Boyer et al., 1998).

Loss of MMR leads to resistance to a wide variety of conventional cytotoxic agents, including alkylating agents, cisplatin, doxorubicin, 6-thioguanine, etc. (see, e.g., Fink et al., 1998). Chemotherapy of ovarian and breast tumours has been reported to select for cells defective in expression of MMR proteins (see, e.g., Mackay et al., 2000; Strathdee et al., 1999). It has been demonstrated that MMR is necessary for engagement of apoptosis (see, e.g., Anthoney et al., 1996). Downstream-signalling events from DNA damage such as phosphorylation of p53 (see, e.g., Duckett et al., 1999) and activation of p73 (see, e.g., Gong et al., 1999) are reduced or absent in MMR defective cells. Thus, it has been argued that MMR is essential for engaging cell death in response to these cytotoxic agents, and this, together with the role of MMR interacting with sites of persistent DNA damage during DNA replication, may explain the anticancer activity of these cytotoxic agents (see, e.g., Brown, 1999).

### SUMMARY OF THE INVENTION

One aspect of the invention pertains to certain compounds, specifically, certain 2-[3H-thiazol-2-ylidinemethyl]pyridine compounds and analogs thereof, as described herein, and their surprising and unexpected activity as antiproliferative agents.

Another aspect of the invention pertains to a composition comprising a compound as described herein and a pharmaceutically acceptable carrier or diluent.

Another aspect of the present invention pertains to a compound as described herein for use in a method of treatment of the human or animal body by therapy.

Another aspect of the present invention pertains to use of a compound, as described herein, in the manufacture of a medicament for use in treatment.

Another aspect of the present invention pertains to a method of treatment comprising administering to a patient in need of treatment a therapeutically effective amount of a compound as described herein, preferably in the form of a pharmaceutical composition.

In one embodiment (e.g., of use in methods of therapy, of use in the manufacture of medicaments, of methods of treatment), the treatment is treatment of a proliferative condition (e.g., cancer).

As will be appreciated by one of skill in the art, features and preferred embodiments of one aspect of the invention will also pertain to other aspect of the invention.

### BRIEF DESCRIPTION OF THE DRAWINGS

FIG. 1 shows two graphs, one of relative tumour volume versus time (days), and one of relative body weight versus time (days), for HCT116 human colon tumour cell line grown as xenograft in nude mice. Mice were treated with a single bolus injection on day 0 with (a) control, (b) cisplatin (6 mg/kg) i.p., (c) MMR201 (1 mg/kg) i.v., and (d) MMR201 (2 mg/kg) i.v. Error bars represent standard error of the mean.

FIG. 2 shows two graphs, one of relative tumour volume versus time (days), and one of relative body weight versus time (days), for A2780/cp70 human ovarian, cisplatin resistant, cell line grown as xenograft in nude mice. Mice were treated with a single bolus injection on day 0 with (a) control, (b) MMR201 (1 mg/kg) i.v., and (c) cisplatin (6 mg/kg), i.p. Error bars represent standard error of the mean.

FIG. 3 shows two graphs, one of relative tumour volume versus time (days), and one of relative body weight versus time (days), for A2780 human ovarian tumour cell line grown as xenograft in nude mice. Mice were treated with a single bolus injection on day 0 with (a) control, (b) MMR201 (2 mg/kg) i.v., and (c) cisplatin (6 mg/kg), i.p. Error bars represent standard error of the mean.

FIG. 4 shows two graphs, one of relative tumour volume versus time (days), and one of relative body weight versus time (days), for A2780/cp70 human ovarian, cisplatin resistant, cell line grown as xenograft in nude mice. Mice were treated with a bolus injection of MMR201 (2 mg/kg) i.v. on each of days 0, 2, 4, and 6. Error bars represent standard error of the mean.

FIG. 5 shows one graphs of relative tumour volume versus time (days) for A2780/cp70 human ovarian, cisplatin resistant, cell line grown as xenograft in nude mice. Mice were treated with a single bolus injection of MMR201 (1 mg/kg, 2 mg/kg or 3 mg/kg) i.v. on day 0. Error bars represent standard error of the mean.

FIG. 6 shows two graphs, one of relative tumour volume versus time (days), and one of relative body weight versus time (days), for A2780/cp70 human ovarian, cisplatin resistant, cell line grown as xenograft in nude mice. Mice were treated with a single bolus injection of MMR201 (2 mg/kg) i.v. or MMR203 (2 mg/kg) i.v. on day 0. Error bars represent standard error of the mean.

FIG. 7 shows two graphs, one of relative tumour volume versus time (days), and one of relative body weight versus time (days), for A2780/cp70 human ovarian, cisplatin resistant, cell line grown as xenograft in nude mice. Mice were treated with a single bolus injection of MMR203 (3 mg/kg) i.v. on day 0. Error bars represent standard error of the mean.

### DETAILED DESCRIPTION OF THE INVENTION

One aspect of the present invention pertains to compounds which may be described as “2-[3H-thiazol-2-ylidinemethyl]pyridine compounds and analogs thereof,” and their surprising and unexpected activity as antiproliferative agents.

### Compounds

One aspect of the present invention pertains to compounds of the following formula:

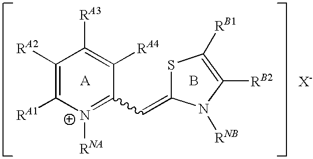

wherein:
R^{NA} and X⁻ may be present, or R^{NA} and X⁻ may be absent;
if R^{NA} and X⁻ are present, then:
   the nitrogen atom of the A-ring bears a positive charge;
if R^{NA} and X⁻ are absent, then:
   the nitrogen atom of the A-ring is electrically neutral;
      and wherein:
(1-a) each of R^{A1}, R^{A2}, R^{A3}, and R^{A4} is independently an A-ring monovalent monodentate substituent; or:
(1-b) each of R^{A3} and R^{A4} is independently an A-ring monovalent monodentate substituent, and R^{A1} and R^{A2}, together with the carbon ring atoms of the A-ring to which they are attached, form a D-ring that is fused to the A-ring and which is:
(1-b)(i) a 6-membered carboaromatic ring, or
(1-b)(ii) a 5-membered heteroaromatic ring having exactly 1 heteroatom ring atom, which heteroatom is selected from N, O, and S, or
(1-b)(iii) a 5-membered heteroaromatic ring having exactly 2 heteroatom ring atoms, which heteroatoms are selected from N, O, and S;
and wherein the D-ring is unsubstituted, or is substituted with one or more D-ring substituents, provided that D-ring substituents do not form a further ring; or:
(1-c) each of R^{A1} and R^{A4} is independently an A-ring monovalent monodentate substituent, and R^{A2} and R^{A3}, together with the carbon ring atoms of the A-ring to which they are attached, form an E-ring that is fused to the A-ring and which is:
(1-c)(i) a 6-membered carboaromatic ring, or
(1-c)(ii) a 5-membered heteroaromatic ring having exactly 1 heteroatom ring atom, which heteroatom is selected from N, O, and S, or
(1-c)(iii) a 5-membered heteroaromatic ring having exactly 2 heteroatom ring atoms, which heteroatoms are selected from N, O, and S;
and wherein the E-ring is unsubstituted, or is substituted with one or more E-ring substituents, provided that E-ring substituents do not form a further ring; or:
(1-d) each of R^{A1} and R^{A2} is independently an A-ring monovalent monodentate substituent, and R^{A3} and R^{A4}, together with the carbon ring atoms of the A-ring to which they are attached, form a C-ring that is fused to the A-ring and which is:
(1-d)(i) a 6-membered carboaromatic ring, or
(1-d)(ii) a 5-membered heteroaromatic ring having exactly 1 heteroatom ring atom, which heteroatom is selected from N, O, and S, or
(1-d)(iii) a 5-membered heteroaromatic ring having exactly 2 heteroatom ring atoms, which heteroatoms are selected from N, O, and S;
and wherein the C-ring is unsubstituted, or is substituted with one or more C-ring substituents, provided that C-ring substituents do not form a further ring;
   and wherein:
(2-a) each of R^{B1} and R^{B2} is independently a B-ring monovalent monodentate substituent; or:
(2-b) R^{B1} and R^{B2}, together with the carbon ring atoms of the B-ring to which they are attached, form an F-ring that is fused to the B-ring and which is:
(i) a 5-membered heteroaromatic ring having exactly 1 heteroatom ring atom, which heteroatom is selected from N, O, and S, or
(ii) a 5-membered heteroaromatic ring having exactly 2 heteroatom ring atoms, which heteroatoms are selected from N, O, and S;
and wherein the F-ring is unsubstituted, or is substituted with one or more F-ring substituents, provided that F-ring substituents do not form a further ring; or:
(2-c) R^{B1} and R^{B2}, together with the carbon ring atoms of the B-ring to which they are attached, form an F-ring that is fused to the B-ring and which is a 5-membered heteroaromatic ring having exactly 1 heteroatom ring atom, which heteroatom is selected from N, O, and S,
wherein the F-ring is substituted with two adjacent F-ring substituents which, together with the carbon ring atoms of the F-ring to which they are attached, form a G-ring that is fused to the F-ring and which is:
(2-c)(i) a 6-membered carboaromatic ring, or
(2-c)(ii) a 5-membered heteroaromatic ring having exactly 1 heteroatom ring atom, which heteroatom is selected from N, O, and S, or
(2-c)(iii) a 5-membered heteroaromatic ring having exactly 2 heteroatom ring atoms, which heteroatoms are selected from N, O, and S;
and wherein the G-ring is unsubstituted, or is substituted with one or more G-ring substituents, provided that G-ring substituents do not form a further ring;
   and wherein:
R^{NA}, if present, is independently an A-ring nitrogen substituent;
R^{NB} is independently a B-ring nitrogen substituent;
the linkage joining the A-ring and the B-ring is cis or trans;
and pharmaceutically acceptable salts, solvates, amides, esters, ethers, N-oxides, chemically protected forms, and prodrugs thereof.

For the avoidance of doubt, it should be noted that the F-ring, if present, is a 5-membered heteroaromatic ring, and is NOT a 6-membered ring.

One preferred group of compounds are those wherein, in respect of the “left side” of the compound, case (1-a) or case (1-b)(i) applies.

One preferred group of compounds are those wherein, in respect of the “left side” of the compound, case (1-a) applies.

One preferred group of compounds are those wherein, in respect of the “left side” of the compound, case (1-b)(i) applies.

One preferred group of compounds are those wherein, in respect of the “right side” of the compound, case (2-a), case (2-b)(i), or case (2-c)(i) applies.

One preferred group of compounds are those wherein, in respect of the “right side” of the compound, case (2-b)(i) or case (2-c)(i) applies.

One preferred group of compounds are those wherein, in respect of the “right side” of the compound, case (2-c)(i) applies.

One preferred group of compounds are those wherein, in respect of the “left side” of the compound, case (1-a) or case (1-b)(i) applies; and in respect of the “right side” of the compound, case (2-a), case (2-b)(i), or case (2-c)(i) applies.

One preferred group of compounds are those wherein, in respect of the “left side” of the compound, case (1-a) or case (1-b)(i) applies; and in respect of the “right side” of the compound, case (2-b)(i), or case (2-c)(i) applies.

One preferred group of compounds are those wherein, in respect of the “left side” of the compound, case (1-a) or case (1-b)(i) applies; and in respect of the “right side” of the compound, case (2-c)(i) applies.

One preferred group of compounds are those wherein, in respect of the “left side” of the compound, case (1-b)(i) applies; and in respect of the “right side” of the compound, case (2-a), case (2-b)(i), or case (2-c)(i) applies.

One preferred group of compounds are those wherein, in respect of the “left side” of the compound, case (1-b)(i) applies; and in respect of the “right side” of the compound, case (2-b)(i), or case (2-c)(i) applies.

One preferred group of compounds are those wherein, in respect of the “left side” of the compound, case (1-b)(i) applies; and in respect of the “right side” of the compound, case (2-c)(i) applies.

### Provisos

In one or more aspects of the present invention (e.g., compounds, compositions, etc.), the compounds are optionally as defined herein, but with the proviso is that the compound is not one of the following compounds (collectively denoted (P-01) to (P-15) and (Q-01) to (Q-09)):

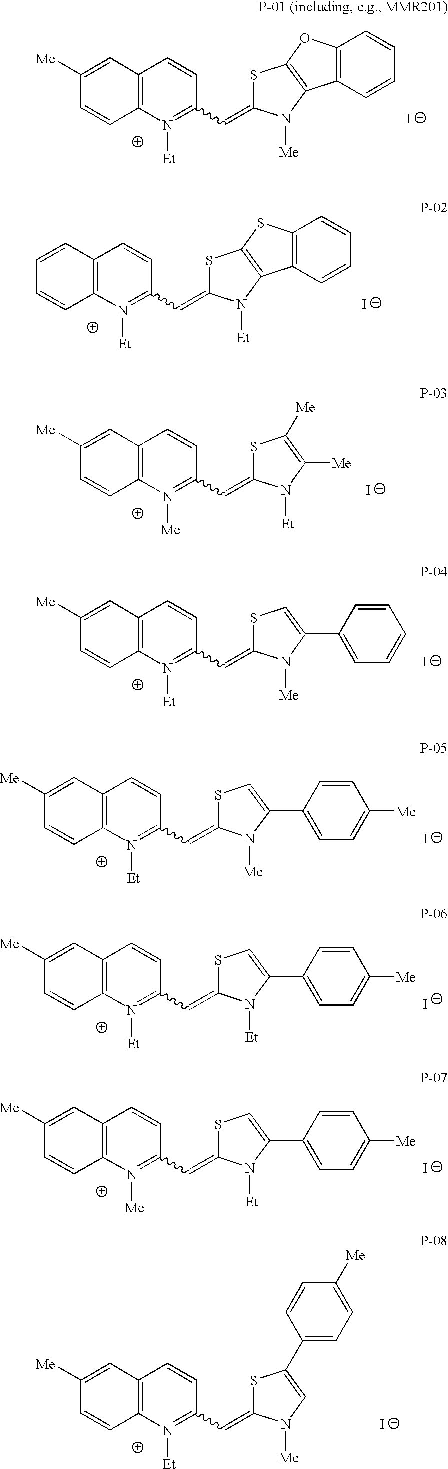

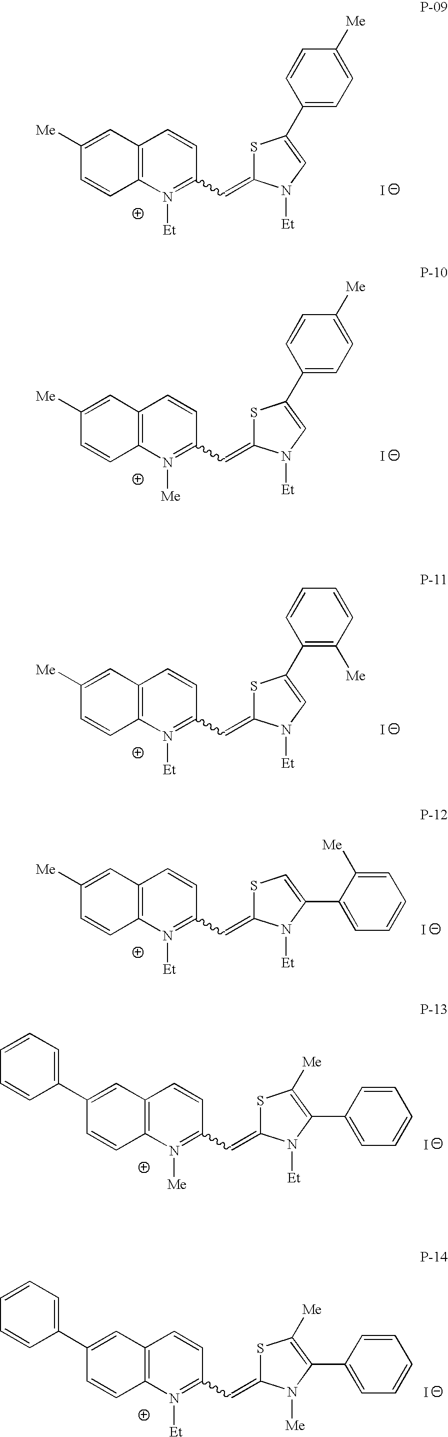

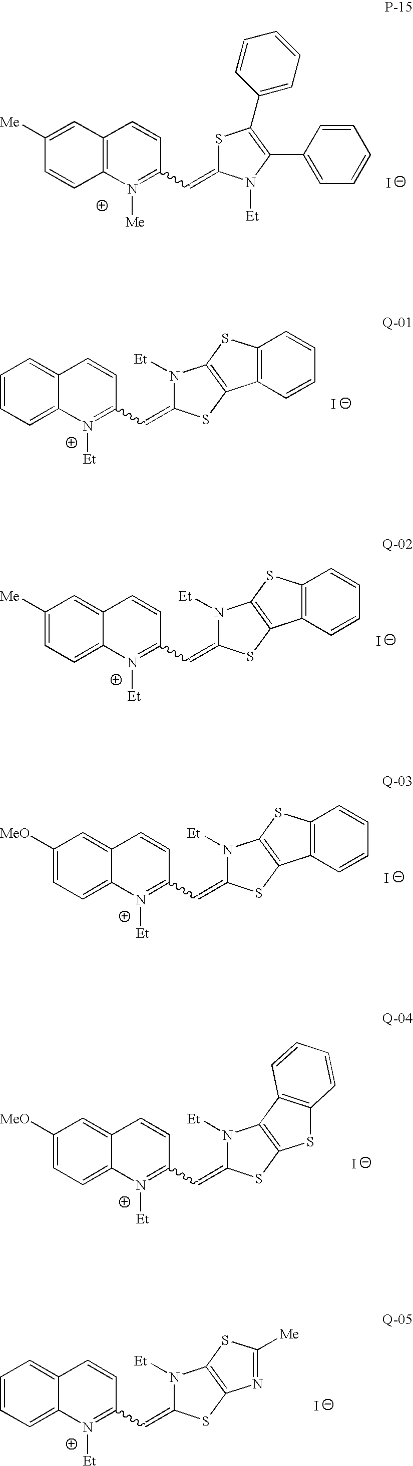

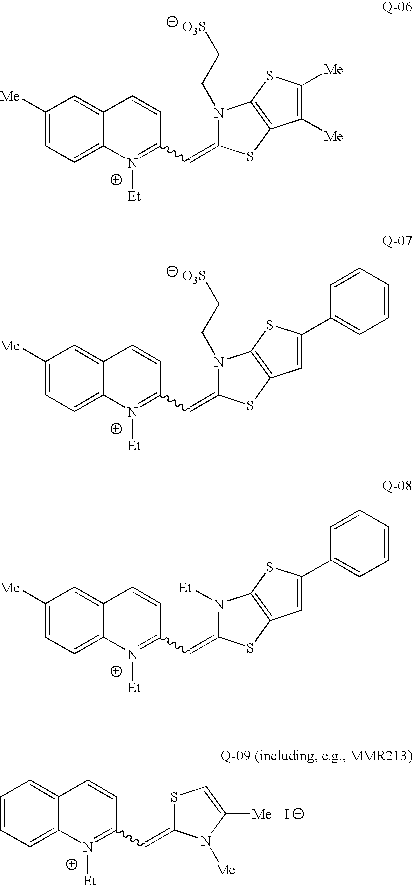

In one or more aspects of the present invention (e.g., compounds for use in therapy, use of compounds in the manufacture of a medicament, methods of treatment, etc.), the compounds are optionally as defined herein, but without the above proviso.

For example, a reference to a particular group of compounds “without the recited proviso” or “without the recited proviso regarding compounds (P-01) to (P-15) and (Q-01) to (Q-09)” (e.g., for use in therapy) is intended to be a reference to the compounds as defined, but wherein the definition no longer includes the indicated proviso. In such cases, it is as if the indicated proviso has been deleted from the definition of compounds, and the definition has been expanded to encompass those compounds which otherwise would have been excluded by the indicated proviso.

### The Linkage Joining the A-Ring and the B-Ring

The linkage joining the A-ring and the B-ring is cis or trans.

In one embodiment, the linkage joining the A-ring and the B-ring is cis.

In one embodiment, the linkage joining the A-ring and the B-ring is trans.

### The A-Ring Nitrogen Substituent

The A-ring nitrogen substituent, R^{NA}, may be present or absent.

If R^{NA} is present, then: X⁻ is also present.

If R^{NA} is absent, then: X⁻ is also absent.

If R^{NA} is present, then: the nitrogen atom of the A-ring bears a positive charge.

If R^{NA} is absent, then: the nitrogen atom of the A-ring is electrically neutral.

If R^{NA} and X⁻ are absent, then the compounds may conveniently be depicted as:

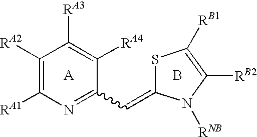

Note that, in such compounds (where R^{NA} and X⁻ are absent), the A-ring nitrogen atom may be protonated, for example, when placed in aqueous solution, to yield compounds where R^{NA} and X⁻ are present, where R^{NA} is —H, and the nitrogen atom of the A-ring bears a positive charge.

### The Pyridinium-Side (Left-Side) of the Compound: The A-Ring etc.

### The Absence of a Ring Fused to the A-Ring

In one embodiment (1-a), each of R^{A1}, R^{A2}, R^{A3}, and R^{A4} is independently a monovalent monodentate substituent; that is, the A-ring is not fused to another ring; that is, there is no D-Ring or E-Ring or C-Ring.

### The D-Ring

In one embodiment (1-b), R^{A3} is independently an A-ring monovalent monodentate substituent, and R^{A1} and R^{A2}, together with the carbon ring atoms of the A-ring to which they are attached, form a D-ring that is fused to the A-ring and which is:
(1-b)(i) a 6-membered carboaromatic ring, or
(1-b)(ii) a 5-membered heteroaromatic ring having exactly 1 heteroatom ring atom, which heteroatom is selected from N, O, and S, or
(1-b)(iii) a 5-membered heteroaromatic ring having exactly 2 heteroatom ring atoms, which heteroatoms are selected from N, O, and S.

The D-ring, if present, is unsubstituted, or is substituted with one or more (i.e., 1, 2, 3, 4, as appropriate) D-ring substituents, for example, one or more monovalent monodentate substituents, as defined herein. Such substituents may be on ring carbon atoms, or on a ring nitrogen atom (e.g., as —NR— or —N⁺(R)═), if present.

D-ring substituents, if present, do not form a further ring, e.g., a further ring fused to the D-ring, a further ring fused both the D-ring and the A-ring, etc. (This does not exclude D-ring substituents that are, or comprise, a ring, e.g., morpholino, benzyl).

Case (1-b)(i):

In one embodiment, the D-ring is a 6-membered carboaromatic ring (i.e., an aromatic ring having 6 ring atoms, each of which is carbon).

In one embodiment, the D-ring is the ring in benzene (C₆).

An example is shown below, wherein w is 0, 1, 2, 3, or 4, and each R^{D} is independently a D-ring substituent (e.g., a monovalent monodentate substituent):

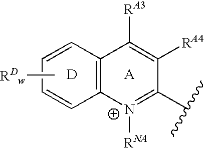

In one embodiment, the moiety:

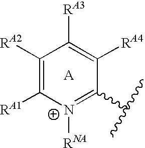

is selected from:

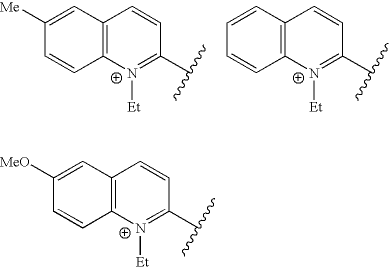

Case (1-b)(ii):

In one embodiment, the D-ring is a 5-membered heteroaromatic ring having exactly 1 heteroatom ring atom, which heteroatom is selected from N, O, and S.

In one embodiment, the D-ring is a 5-membered heteroaromatic ring having exactly 1 heteroatom ring atom, which heteroatom is selected from O and S.

In one embodiment, the D-ring is selected from the rings in: pyrrole (N₁), furan (O₁), and thiophene (S₁).

In one embodiment, the D-ring is selected from the rings in: furan (O₁) and thiophene (S₁).

Some examples are shown below, wherein y is 0, 1, or 2, and each R^{D} is independently a D-ring substituent (e.g., a monovalent monodentate substituent):

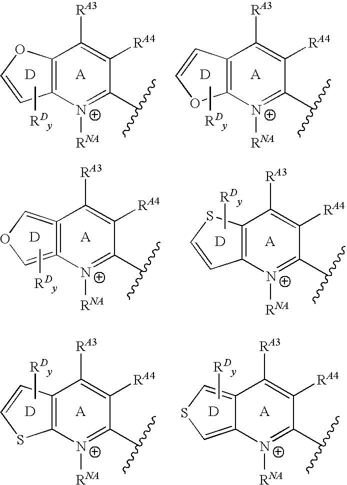

Case (1-b)(iii):

In one embodiment, the D-ring is a 5-membered heteroaromatic ring having exactly 2 heteroatom ring atoms, which heteroatoms are selected from N, O, and S.

In one embodiment, the D-ring is a 5-membered heteroaromatic ring having exactly 2 heteroatom ring atoms, one of which is N and the other of which is selected from O and S.

In one embodiment, the D-ring is selected from the rings in: imidazole (N₂), pyrazole (N₂), oxazole (N₁O₁), isoxazole (N₁O₁), thiazole (N₁S₁), and isothiazole (N₁S₁).

In one embodiment, the D-ring is selected from the rings in: oxazole (N₁O₁), isoxazole (N₁O₁), thiazole (N₁S₁), and isothiazole (N₁S₁).

In one embodiment, the D-ring is selected from the rings in: oxazole (N₁O₁) and thiazole (N₁S₁).

Some examples are shown below, wherein y is 0 or 1, and each R^{D} is independently a D-ring substituent (e.g., a monovalent monodentate substituent):

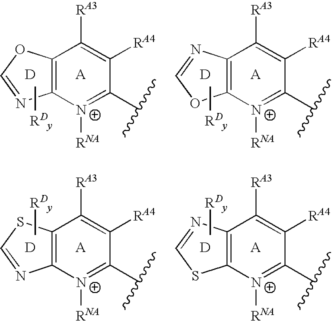

### The E-Ring

In one embodiment (1-c), R^{A1} is independently an A-ring monovalent monodentate substituent, and R^{A2} and R^{A3}, together with the carbon ring atoms of the A-ring to which they are attached, form an E-ring that is fused to the A-ring and which is:
(1-c)(i) a 6-membered carboaromatic ring, or
(1-c)(ii) a 5-membered heteroaromatic ring having exactly 1 heteroatom ring atom, which heteroatom is selected from N, O, and S, or
(1-c)(iii) a 5-membered heteroaromatic ring having exactly 2 heteroatom ring atoms, which heteroatoms are selected from N, O, and S.

The E-ring, if present, is unsubstituted, or is substituted with one or more (i.e., 1, 2, 3, 4, as appropriate) E-ring substituents, for example, one or more monovalent monodentate substituents, as defined herein. Such substituents may be on ring carbon atoms, or on a ring nitrogen atom (e.g., as —NR— or —N⁺(R)═), if present.

E-ring substituents, if present, do not form a further ring, e.g., a further ring fused to the E-ring, a further ring fused both the E-ring and the A-ring, etc. (This does not exclude E-ring substituents that are, or comprise, a ring, e.g., morpholino, benzyl).

Case (1-c)(i):

In one embodiment, the E-ring is a 6-membered carboaromatic ring (i.e., an aromatic ring having 6 ring atoms, each of which is carbon).

In one embodiment, the E-ring is the ring in benzene (C₆).

An example is shown below, wherein w is 0, 1, 2, 3, or 4 and each R^{B} is independently an E-ring substituent (e.g., a monovalent monodentate substituent):

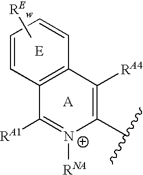

Case (1-c)(ii):

In one embodiment, the E-ring is a 5-membered heteroaromatic ring having exactly 1 heteroatom ring atom, which heteroatom is selected from N, O, and S.

In one embodiment, the E-ring is a 5-membered heteroaromatic ring having exactly 1 heteroatom ring atom, which heteroatom is selected from O and S.

In one embodiment, the E-ring is selected from the rings in: pyrrole (N₁), furan (O₁), and thiophene (S₁).

In one embodiment, the E-ring is selected from the rings in: furan (O₁) and thiophene (S₁).

Some examples are shown below, wherein y is 0, 1, or 2, and each R^{B} is independently an E-ring substituent (e.g., a monovalent monodentate substituent):

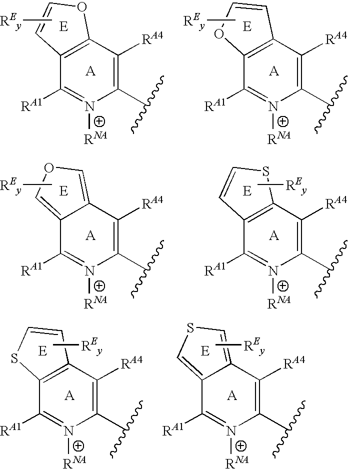

Case (1-c)(iii):

In one embodiment, the E-ring is a 5-membered heteroaromatic ring having exactly 2 heteroatom ring atoms, which heteroatoms are selected from N, O, and S.

In one embodiment, the E-ring is a 5-membered heteroaromatic ring having exactly 2 heteroatom ring atoms, one of which is N and the other of which is selected from O and S.

In one embodiment, the E-ring is selected from the rings in: imidazole (N₂), pyrazole (N₂), oxazole (N₁O₁), isoxazole (N₁O₁), thiazole (N₁S₁), and isothiazole (N₁S₁).

In one embodiment, the E-ring is selected from the rings in: oxazole (N₁O₁), isoxazole (N₁O₁), thiazole (N₁S₁), and isothiazole (N₁S₁).

In one embodiment, the E-ring is selected from the rings in: oxazole (N₁O₁) and thiazole (N₁S₁).

Some examples are shown below, wherein y is 0 or 1, and each R^{B} is independently an E-ring substituent (e.g., a monovalent monodentate substituent):

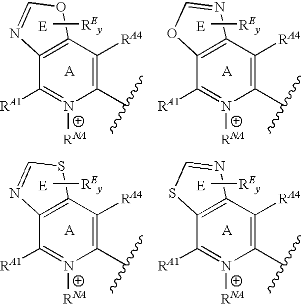

### The C-Ring

In one embodiment (1-d), each of R^{A1} and R^{A2} is independently an A-ring monovalent monodentate substituent, and R^{A3} and R^{A4}, together with the carbon ring atoms of the A-ring to which they are attached, form a C-ring that is fused to the A-ring and which is:
(1-d)(i) a 6-membered carboaromatic ring, or
(1-d)(ii) a 5-membered heteroaromatic ring having exactly 1 heteroatom ring atom, which heteroatom is selected from N, O, and S, or
(1-d)(iii) a 5-membered heteroaromatic ring having exactly 2 heteroatom ring atoms, which heteroatoms are selected from N, O, and S.

The C-ring, if present, is unsubstituted, or is substituted with one or more (i.e., 1, 2, 3, 4, as appropriate) C-ring substituents, for example, one or more monovalent monodentate substituents, as defined herein. Such substituents may be on ring carbon atoms, or on a ring nitrogen atom (e.g., as —NR— or —N⁺(R)═), if present.

C-ring substituents, if present, do not form a further ring, e.g., a further ring fused to the C-ring, a further ring fused both the C-ring and the A-ring, etc. (This does not exclude C-ring substituents that are, or comprise, a ring, e.g., morpholino, benzyl).

Case (1-d)(i):

In one embodiment, the C-ring is a 6-membered carboaromatic ring (i.e., an aromatic ring having 6 ring atoms, each of which is carbon).

In one embodiment, the C-ring is the ring in benzene (C₆).

An example is shown below, wherein w is 0, 1, 2, 3, or 4 and each R^{C} is independently a C-ring substituent (e.g., a monovalent monodentate substituent):

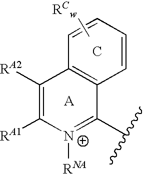

Case (1-d)(ii):

In one embodiment, the C-ring is a 5-membered heteroaromatic ring having exactly 1 heteroatom ring atom, which heteroatom is selected from N, O, and S.

In one embodiment, the C-ring is a 5-membered heteroaromatic ring having exactly 1 heteroatom ring atom, which heteroatom is selected from O and S.

In one embodiment, the C-ring is selected from the rings in: pyrrole (N₁), furan (O₁), and thiophene (S₁).

In one embodiment, the C-ring is selected from the rings in: furan (O₁) and thiophene (S₁).

Some examples are shown below, wherein y is 0, 1, or 2, and each R^{C} is independently a C-ring substituent (e.g., a monovalent monodentate substituent):

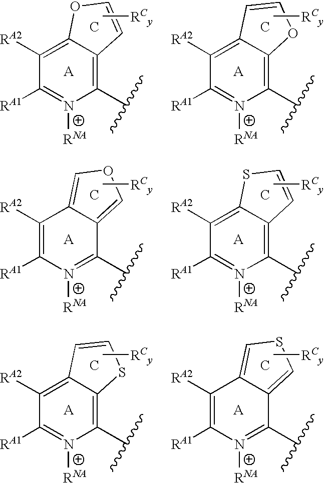

Case (1-d)(iii):

In one embodiment, the C-ring is a 5-membered heteroaromatic ring having exactly 2 heteroatom ring atoms, which heteroatoms are selected from N, O, and S.

In one embodiment, the C-ring is a 5-membered heteroaromatic ring having exactly 2 heteroatom ring atoms, one of which is N and the other of which is selected from O and S.

In one embodiment, the C-ring is selected from the rings in: imidazole (N₂), pyrazole (N₂), oxazole (N₁O₁), isoxazole (N₁O₁), thiazole (N₁S₁), and isothiazole (N₁S₁).

In one embodiment, the C-ring is selected from the rings in: oxazole (N₁O₁), isoxazole (N₁O₁), thiazole (N₁S₁), and isothiazole (N₁S₁).

In one embodiment, the C-ring is selected from the rings in: oxazole (N₁O₁) and thiazole (N₁S₁).

Some examples are shown below, wherein y is 0 or 1, and each R^{C} is independently a C-ring substituent (e.g., a monovalent monodentate substituent):

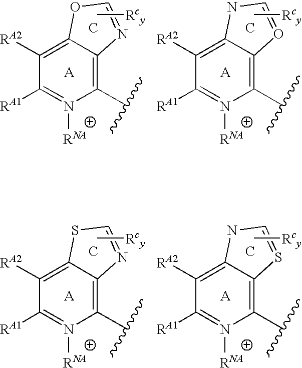

### The Thiazolium-Side (Right-Side) of the Compound: The B-Ring etc.

### The Absence of a Ring Fused to the B-Ring

In one embodiment (2-a), each of R^{B1} and R^{B2} is independently a monovalent monodentate substituent; that is, the B-ring is not fused to another ring; that is, there is no F-Ring.

Alternatively, in one embodiment, case (2-a) does not apply.

### The F-Ring

For the avoidance of doubt, it should be noted that the F-ring, if present, is a 5-membered heteroaromatic ring, and is NOT a 6-membered ring.

In one embodiment (2-b), R^{B1} and R^{B2}, together with the carbon ring atoms of the B-ring to which they are attached, form an F-ring that is fused to the B-ring and which is:
(2-b)(i) a 5-membered heteroaromatic ring having exactly 1 heteroatom ring atom, which heteroatom is selected from N, O, and S, or
(2-b)(ii) a 5-membered heteroaromatic ring having exactly 2 heteroatom ring atoms, which heteroatoms are selected from N, O, and S.

The F-ring, if present, is unsubstituted, or is substituted with one or more (i.e., 1, 2, as appropriate) F-ring substituents, for example, one or more monovalent monodentate substituents, as defined herein. Such substituents may be on ring carbon atoms, or on a ring nitrogen atom (e.g., as —NR— or —N⁺(R)═), if present.

In this embodiment, F-ring substituents, if present, do not form a further ring, e.g., a further ring fused to the F-ring, a further ring fused both the F-ring and the B-ring, etc. (This does not exclude F-ring substituents that are, or comprise, a ring, e.g., morpholino, benzyl).

(This is in contrast to the embodiments described below, wherein the F-ring is substituted with two adjacent F-ring substituents which, together with the carbon ring atoms of the F-ring to which they are attached, form a G-ring that is fused to the F-ring. See below.)

Case (2-b)(i):

In one embodiment, R^{B1} and R^{B2}, together with the carbon ring atoms of the B-ring to which they are attached, form an F-ring that is fused to the B-ring and which is:
a 5-membered heteroaromatic ring having exactly 1 heteroatom ring atom, which heteroatom is selected from N, O, and S.

In one embodiment, the F-ring is selected from the rings in: pyrrole (N₁), furan (O₁), and thiophene (S₁).

In one embodiment, the F-ring is selected from the rings in: furan (O₁) and thiophene (S₁).

In one embodiment, the F-ring is the ring in furan (O₁).

In one embodiment, the F-ring is the ring in thiophene (S₁).

Some examples are shown below, wherein x is 0, 1, or 2, and each R^{F} is independently an F-ring substituent (e.g., a monovalent monodentate substituent):

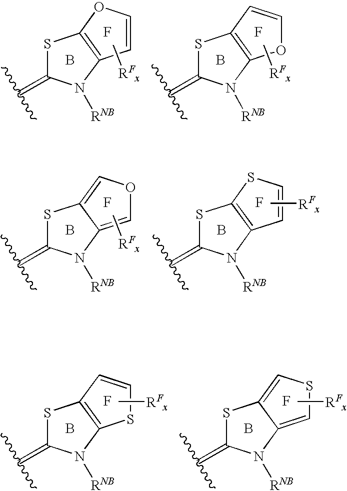

Case (2-b)(ii):

In one embodiment, R^{B1} and R^{B2}, together with the carbon ring atoms of the B-ring to which they are attached, form an F-ring that is fused to the B-ring and which is:
a 5-membered heteroaromatic ring having exactly 2 heteroatom ring atoms, which heteroatoms are selected from N, O, and S.

In one embodiment, the F-ring is a 5-membered heteroaromatic ring having exactly 2 heteroatom ring atoms, one of which is N and the other of which is selected from O and S.

In one embodiment, the F-ring is selected from the rings in: imidazole (N₂), pyrazole (N₂), oxazole (N₁O₁), isoxazole (N₁O₁), thiazole (N₁S₁), and isothiazole (N₁S₁).

In one embodiment, the F-ring is selected from the rings in: oxazole (N₁O₁), isoxazole (N₁O₁), thiazole (N₁S₁), and isothiazole (N₁S₁).

In one embodiment, the F-ring is selected from the rings in: oxazole (N₁O₁) and thiazole (N₁S₁).

Some examples are shown below, wherein x is 0 or 1, and each RF is independently an F-ring substituent (e.g., a monovalent monodentate substituent):

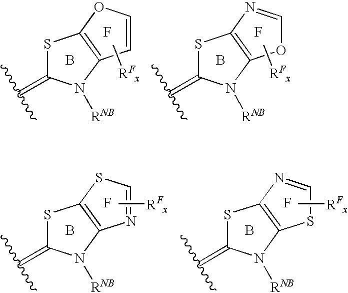

### The Absence of a Further Ring Fused to the F-Ring

In one embodiment, when the F-ring is present, the F-ring is not fused to another ring, other than the B-ring; that is, there is no G-ring. See above.

If an F-ring is present, and if that F-ring is a 5-membered heteroaromatic ring having exactly 1 heteroatom ring atom (e.g., pyrrole (N₁), furan (O₁), and thiophene (S₁)), then two adjacent F-ring substituents, together with the carbon ring atoms of the F-ring to which they are attached, optionally form a G-ring that is fused to the F-ring. See below.

### The G-Ring

In one embodiment (2-c), R^{B1} and R^{B2}, together with the carbon ring atoms of the B-ring to which they are attached, form an F-ring that is fused to the B-ring and which is a 5-membered heteroaromatic ring having exactly 1 heteroatom ring atom, which heteroatom is selected from N, O, and S (e.g., pyrrole (N₁), furan (O₁), and thiophene (S₁)),
wherein the F-ring is substituted with two adjacent F-ring substituents which, together with the carbon ring atoms of the F-ring to which they are attached, form a G-ring that is fused to the F-ring and which is:
(2-c)(i) a 6-membered carboaromatic ring, or
(2-c)(ii) a 5-membered heteroaromatic ring having exactly 1 heteroatom ring atom, which heteroatom is selected from N, O, and S, or
(2-c)(iii) a 5-membered heteroaromatic ring having exactly 2 heteroatom ring atoms, which heteroatoms are selected from N, O, and S.

The G-ring, if present, is unsubstituted, or is substituted with one or more (i.e., 1, 2, 3, 4, as appropriate) G-ring substituents, for example, one or more monovalent monodentate substituents, as defined herein. Such substituents may be on ring carbon atoms, or on a ring nitrogen atom (e.g., as —NR— or —N⁺(R)═), if present.

G-ring substituents, if present, do not form a further ring, e.g., a further ring fused to the G-ring, a further ring fused both the G-ring and the F-ring, etc. (This does not exclude G-ring substituents that are, or comprise, a ring, e.g., morpholino, benzyl).

Case (2-c)(i):

In one embodiment, the G-ring is a 6-membered carboaromatic ring (i.e., an aromatic ring having 6 ring atoms, each of which is carbon).

In one embodiment, the G-ring is the ring in benzene (C₆).

In one embodiment, the F-ring is the ring in furan (O₁), and the G-ring is the ring in benzene (C₆).

In one embodiment, the F-ring is the ring in thiophene (S₁), and the G-ring is the ring in benzene (C₆).

Some examples are shown below, wherein z is 0, 1, 2, 3, or 4 and each R^{G} is independently a G-ring substituent (e.g., a monovalent monodentate substituent):

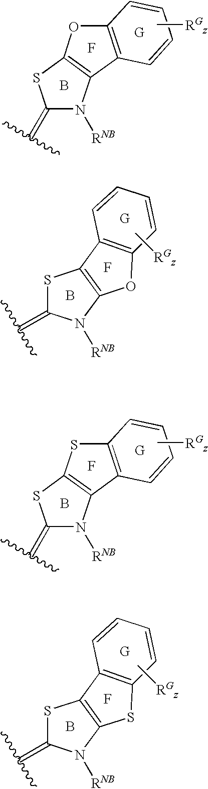

Case (2-c)(ii):

In one embodiment, the G-ring is a 5-membered heteroaromatic ring having exactly 1 heteroatom ring atom, which heteroatom is selected from N, O, and S.

In one embodiment, the G-ring is a 5-membered heteroaromatic ring having exactly 1 heteroatom ring atom, which heteroatom is selected from O and S.

In one embodiment, the G-ring is selected from the rings in: pyrrole (N₁), furan (O₁), and thiophene (S₁).

In one embodiment, the G-ring is selected from the rings in: furan (O₁) and thiophene (S₁).

Some examples are shown below, wherein v is 0, 1, or 2, each of X¹ and X² is independently —O— or —S—, and each R^{G} is independently a G-ring substituent (e.g., a monovalent monodentate substituent):

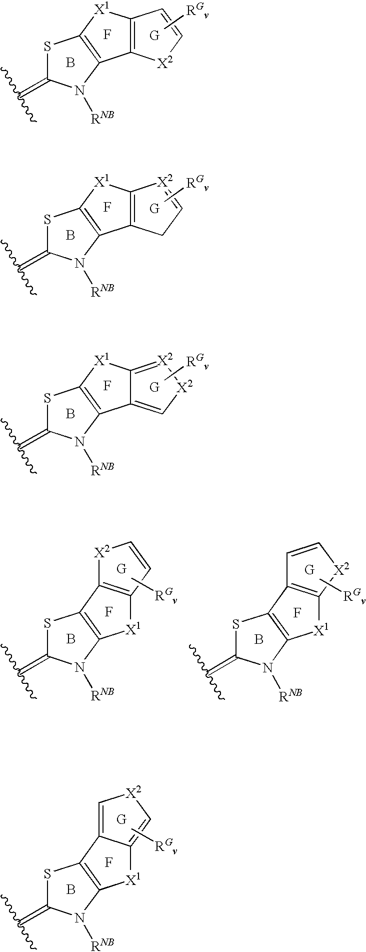

In one embodiment, X¹ is independently —O— and X² is independently —O—.

In one embodiment, X¹ is independently —S— and X² is independently —S—.

In one embodiment, X¹ is independently —O— and X² is independently —S—.

In one embodiment, X¹ is independently —S— and X² is independently —O—.

Case (2-c)(iii):

In one embodiment, the G-ring is a 5-membered heteroaromatic ring having exactly 2 heteroatom ring atoms, which heteroatoms are selected from N, O, and S.

In one embodiment, the G-ring is a 5-membered heteroaromatic ring having exactly 2 heteroatom ring atoms, one of which is N and the other of which is selected from O and S.

In one embodiment, the G-ring is selected from the rings in: imidazole (N₂), pyrazole (N₂), oxazole (N₁O₁), isoxazole (N₁O₁), thiazole (N₁S₁), and isothiazole (N₁S₁).

In one embodiment, the G-ring is selected from the rings in: oxazole (N₁O₁), isoxazole (N₁O₁), thiazole (N₁S₁), and isothiazole (N₁S₁).

In one embodiment, the G-ring is selected from the rings in: oxazole (N₁O₁) and thiazole (N₁S₁).

Some examples are shown below, wherein v is 0, 1, or 2, each X¹ is independently —O— or —S—, and each R^{G} is independently a G-ring substituent (e.g., a monovalent monodentate substituent):

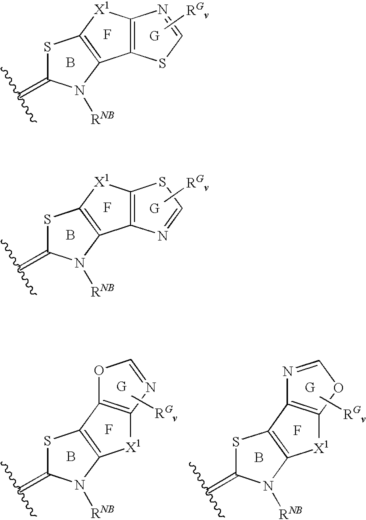

In one embodiment, X¹ is independently —O—.

In one embodiment, X¹ is independently —S—.

### Combinations

All plausible combinations of the embodiments described above are explicitly disclosed herein as if each combination was specifically and individually recited.

For example, any embodiment pertaining to the “left side” of the compound may be combined with any embodiment pertaining to the “right side” of the compound, and each such combination is explicitly disclosed herein as if it was individually recited.

### Additional Substituents on the Rings

Each of the C-ring, D-ring, E-ring, F-ring, and G-ring, if present, is unsubstituted, or is substituted with one or more substituents (e.g., C-ring substituents, D-ring substitutents, etc.) (e.g., R^{C}, R^{D}, R^{B}, R^{F}, R^{G}). In one embodiment, each of these one or more substituents, if present, is a monovalent monodentate substituent or oxo (i.e., ═O).

In one embodiment, each D-ring substituent (e.g., R^{D}), if present, is independently a monovalent monodentate substituent or oxo.

In one embodiment, each D-ring substituent (e.g., R^{D}), if present, is independently a monovalent monodentate substituent.

In one embodiment, each E-ring substituent (e.g., R^{B}), if present, is independently a monovalent monodentate substituent or oxo.

In one embodiment, each E-ring substituent (e.g., R^{B}), if present, is independently a monovalent monodentate substituent.

In one embodiment, each C-ring substituent (e.g., R^{C}), if present, is independently a monovalent monodentate substituent or oxo.

In one embodiment, each C-ring substituent (e.g., R^{C}), if present, is independently a monovalent monodentate substituent.

In one embodiment, each F-ring substituent (e.g., R^{F}), if present, is independently a monovalent monodentate substituent or oxo.

In one embodiment, each F-ring substituent (e.g., R^{F}), if present, is independently a monovalent monodentate substituent.

In one embodiment, each G-ring substituent (e.g., R^{G}), if present, is independently a monovalent monodentate substituent or oxo.

In one embodiment, each G-ring substituent (e.g., R^{G}), if present, is independently a monovalent monodentate substituent.

### The Monovalent Mondentate Substituents

Various embodiments include one or more monovalent monodentate substituents.

In one embodiment, each monovalent monodentate substituent is independently selected from:

### (1) —H;

### (2) —F; —Cl; —Br; —I;

### (3) —OH; —OR;

### (4) —SH; —SR;

### (5) —NO₂;

### (6) —C(═O)R;

### (7) —C(═O)OH; —C(═O)OR;

### (8) —C(═O)NH₂; —C(═O)NHR; —C(═O)NR₂; —C(═O)NR^{N1}R^{N2};

### (9) —NH₂; —NHR; —NR₂; —NR^{N1}R^{N2};

### (10) —NHC(═O)H; —NRC(═O)H; —NHC(═O)R; —NRC(═O)R;

### (11) —R;

### (12) —R^{Alk}—OH; —R^{Alk}—OR

### (13) —R^{Alk}—NH₂; —R^{Alk}—NHR; —R^{Alk}—NR₂; —R^{Alk}—NR^{N1}R^{N2};

### (14) —R^{Alk}—C(═O)OH; —R^{Alk}—C(═O)OR;

(15) —R^{Alk}—C(═O)NH₂; —R^{Alk}—C(═O)NHR; —R^{Alk}—C(═O)NR₂; —R^{Alk}—C(═O)NR^{N1}R^{N2};

### (16) -J-R^{Alk}—OH; -J-R^{Alk}—OR

### (17) -J-R^{Alk}—NH₂; -J-R^{Alk}—NH R; -J-R^{Alk}—NR₂; -J-R^{Alk}—NR^{N1}R^{N2};

### (18) -J-R^{Alk}—C(═O)OH; -J-R^{Alk}—C(═O)OR;

### (19) -J-R^{Alk}—C(═O)NH₂; -J-R^{Alk}—C(═O)NHR;

### (20) -J-R^{Alk}—C(═O)NR₂; -J-R^{Alk}—C(═O)NR^{N1}R^{N2};

wherein each J is independently selected from —O—, —NH—, or —NR—;
wherein each R is independently selected from:
   (a) unsubstituted aliphatic C₁₋alkyl; substituted aliphatic C₁₋₆alkyl;
   (b) unsubstituted aliphatic C₂₋₆alkenyl; substituted aliphatic C₂₋₆alkenyl;
   (c) unsubstituted C₃₋₆cycloalkyl; substituted C₃₋₆cycloalkyl;
   (d) unsubstituted C₆₋₁₀carboaryl; substituted C₆₋₁₀carboaryl;
   (e) unsubstituted C₅₋₁₀heteroaryl; substituted C₅₋₁₀heteroaryl;
   (f) unsubstituted C₆₋₁₀carboaryl-aliphatic C₁₋₄alkyl;
   (g) substituted C₆₋₁₀carboaryl-aliphatic C₁₋₄alkyl;
   (h) unsubstituted C₅₋₁₀heteroaryl-aliphatic C₁₋₄alkyl;
   (i) substituted C₅₋₁₀heteroaryl-aliphatic C₁₋₄alkyl;
wherein each R^{Alk} is independently selected from:
   (I) unsubstituted aliphatic C₁₋₆alkylene; substituted aliphatic C₁₋₆alkylene;
   (II) unsubstituted aliphatic C₂₋₆alkenylene; substituted aliphatic C₂₋₆alkenylene;
   (III) unsubstituted C₃₋₆cycloalkylene; substituted C₃₋₆cycloalkylene;
wherein in each group —NR^{N1}R^{N2}, independently, R^{N1} and R^{N2} taken together with the nitrogen atom to which they are attached form a ring having from 3 to 7 ring atoms.

Examples of groups —NR^{N1}R^{N2}, wherein R^{N1} and R^{N2} taken together with the nitrogen atom to which they are attached form a ring having from 3 to 7 ring atoms, include: pyrrolidino, piperidino, piperazino, morpholino, and substituted forms, such as N-substituted forms, such as N-methyl piperazino.

In one embodiment, each J is independently —O—, —NH—, or —NMe-

In one embodiment, each J is independently —O—.

In one embodiment, each J is independently —NH— or —NMe-.

In one embodiment, each J is independently —NH—.

In one embodiment, each R is independently selected from:

(a) unsubstituted aliphatic C₁₋₆alkyl; substituted aliphatic C₁₋₆alkyl;
(b) unsubstituted aliphatic C₂₋₆alkenyl; substituted aliphatic C₂₋₆alkenyl;
(c) unsubstituted C₃₋₆cycloalkyl; substituted C₃₋₆cycloalkyl.

In one embodiment, each R is independently selected from:

(a) unsubstituted aliphatic C₁₋₆alkyl; substituted aliphatic C₁₋₆alkyl.

In one embodiment, each R is independently selected from: -Me, -Et, -nPr, and -iPr.

In one embodiment, each R is independently selected from: -Me and -Et.

In one embodiment, the C₁₋alkyl group is a C₁₋₄alkyl group.

In one embodiment, the C₂₋₆alkenyl group is a C₂₋₄alkenyl group.

In one embodiment, the C₃₋₆cycloalkyl group is a C₃₋₄cycloalkyl group.

Examples of unsubstituted aliphatic C₁₋₆alkyl groups include: methyl, ethyl, n-propyl, iso-propyl, n-butyl, iso-butyl, sec-butyl, tert-butyl, n-pentyl, iso-pentyl, tert-pentyl, neo-pentyl, hexyl, iso-hexyl.

Examples of unsubstituted aliphatic C₂₋₆alkenyl groups include: propen-1-yl, propen-2-yl, buten-1-yl, buten-2-yl, buten-3-yl.

Examples of unsubstituted C₃₋₆cycloalkyl groups include: cyclopropyl, cyclopropyl-methyl, cyclobutyl, cyclopentyl, cyclohexyl.

In one embodiment, the C₆₋₁₀carboaryl group is a C₆carboaryl group.

In one embodiment, the C₅₋₁₀heteroaryl group is a C₅₋₆heteroaryl group.

In one embodiment, the C₆₋₁₀carboaryl-aliphatic C₁₋₄alkyl group is a C₆carboaryl-C₁₋₂alkyl group.

In one embodiment, the C₆₋₁₀carboaryl-aliphatic C₁₋₄alkyl group is a C₆carboaryl-(CH₂)ₚ group, where p is 1 or 2.

In one embodiment, the C₅₋₁₀heteroaryl-aliphatic C₁₋₄alkyl group is a C₅₋₆heteroaryl-C₁₋₂alkyl group.

In one embodiment, the C₅₋₁₀heteroaryl-aliphatic C₁₋₄alkyl group is a C₅₋₁₀heteroaryl-(CH₂)ₚ group, where p is 1 or 2.

Examples of unsubstituted C₆₋₁₀carboaryl groups include: phenyl, naphthyl.

Examples of unsubstituted C₅₋₁₀heteroaryl groups include: pyrrolyl, thienyl, furyl, imidazolyl, oxazolyl, isoxazolyl, thiazolyl, isothiazolyl, pyrazolyl, pyridyl, pyrazinyl, pyrimidinyl, pyridazinyl.

Examples of unsubstituted C₆₋₁₀carboaryl-aliphatic C₁₋₄alkyl groups include: benzyl, phenylethyl.

Examples of unsubstituted C₅₋₁₀heteroaryl-aliphatic C₁₋₄alkyl groups include: pyridyl-methyl, isothiazolyl-methyl.

In one embodiment, each R^{Alk} is independently selected from:
unsubstituted aliphatic C₁₋₆alkylene;
unsubstituted aliphatic C₂₋₆alkenylene;
unsubstituted C₃₋₆cycloalkylene.

In one embodiment, each R^{Alk} is independently selected from:
unsubstituted aliphatic C₁₋₆alkylene; substituted aliphatic C₁₋₆alkylene;

In one embodiment, each R^{Alk} is independently selected from:

unsubstituted aliphatic C₁₋₆alkylene.

In one embodiment, the C₁₋₆alkylene group is a C₁₋₄alkylene group.

In one embodiment, the C₁₋₆alkylene group is a C₂₋₃alkylene group.

In one embodiment, the C₂₋₆alkenylene group is a C₂₋₄alkenylene group.

In one embodiment, the C₂₋₆alkenylene group is a C₂₋₃alkenylene group.

In one embodiment, the C₃₋₆cycloalkylene group is a C₃₋₄cycloalkylene group.

Examples of unsubstituted aliphatic C₁₋₆alkylene groups include:

—(CH₂)—, —(CH₂)₂—, —(CH₂)₃—, —(CH₂)₄—, —CH(CH₃)CH₂—, —CH(CH₃)CH₂CH₂—.

Examples of unsubstituted aliphatic C₂₋₆alkenylene groups include:

### —CH═CH—, —CH═CH—CH₂—, —CH═CH—CH₂CH₂—, —CH═CH—CH═CH—.

Examples of unsubstituted C₃₋₆cycloalkylene groups include: cyclopropylene, cyclobutylene, cyclopentylene, cyclohexylene.

In one embodiment, each R^{Alk} is independently —(CH₂)ₘ—, wherein m is independently 1, 2, 3, 4, 5, or 6. In one embodiment, m is 2, 3, 4, 5, or 6. In one embodiment, m is 2, 3, or 4. In one embodiment, m is 2 or 3.

For example, in one embodiment, —R^{Alk}—OH is —(CH₂)ₘOH.

For example, in one embodiment, —R^{Alk}—OR is —(CH₂)ₘOR.

For example, in one embodiment, —R^{Alk}—NH₂ is —(CH₂)ₘNH₂.

For example, in one embodiment, —R^{Alk}—NHR is —(CH₂)ₘNHR.

For example, in one embodiment, —R^{Alk}—NR₂ is —(CH₂)ₘNR₂.

For example, in one embodiment, —R^{Alk}—NR^{N1}R^{N2} is —(CH₂)ₘNR^{N1}R^{N2}.

For example, in one embodiment, —R^{Alk}—C(═O)OH is —(CH₂)ₘC(═O)OH.

For example, in one embodiment, —R^{Alk}—C(═O)OR is —(CH₂)ₘC(═O)OR.

For example, in one embodiment, —R^{Alk}—C(═O)NH₂ is —(CH₂)ₘC(═O)NH₂.

For example, in one embodiment, —R^{Alk}—C(═O)NHR is —(CH₂)ₘC(═O)NHR.

For example, in one embodiment, —R^{Alk}—C(═O)NR₂ is —(CH₂)ₘC(═O)NR₂.

For example, in one embodiment, —R^{Alk}—C(═O)NR^{N1}R^{N2} is —(CH₂)ₘC(═O)NR^{N1}R^{N2}.

In one embodiment, optional substituents (e.g., on aliphatic C₁₋₆alkyl, aliphatic C₁₋₆alkenyl, C₃₋₆cycloalkyl, C₆₋₁₀-carboaryl, C₅₋₁₀heteroaryl, C₆₋₁₀-carboaryl-aliphatic C₁₋₄alkyl, C₅₋₁₀heteroaryl-aliphatic C₁₋₄alkyl, aliphatic C₁₋₆alkylene, aliphatic C₂₋₆alkenylene, C₃₋₆cycloalkylene) are independently selected from substituents as defined in (1) to (20) above.

In one embodiment, they are independently selected from substituents as defined in (1) to (11) above.

In one embodiment, each monovalent monodentate substituent is independently selected from:
—H;
—F; —Cl; —Br; —I;
—OH;
—OMe, —OEt, —O(nPr), —O(iPr);
—SH;
—SMe, —SEt, —S(nPr), —S(iPr);
—NO₂;
—C(═O)Me, —C(═O)Et, —C(═O)(nPr), —C(═O)(iPr);
—C(═O)OH;
—C(═O)OMe, —C(═O)OEt, —C(═O)O(nPr), —C(═O)O(iPr);
—C(═O)NH₂;
—C(═O)NHMe, —C(═O)NHEt, —C(═O)NH(nPr), —C(═O)NH(iPr);
—C(═O)NMe₂, —C(═O)NEt₂, —C(═O)N(nPr)₂, —C(═O)N(iPr)₂;
—C(═O)-pyrrolidino, —C(═O)-piperidino, —C(═O)-piperazino, —C(═O)-morpholino;
—NH₂;
—NHMe, —NHEt, —NH(nPr), —NH(iPr);
—NMe₂, —NEt₂, —N(nPr)₂, —N(iPr)₂;
pyrrolidino, piperidino, piperazino, morpholino;
—NHC(═O)H;
—NMeC(═O)H, —NEtC(═O)H, —N(nPr)C(═O)H, —N(iPr)C(═O)H;
—NHC(═O)Me, —NHC(═O)Et, —NHC(═O)(nPr), —NHC(═O)(iPr);
—NMeC(═O)Me, —NEtC(═O)Me, —N(nPr)C(═O)Me, —N(iPr)C(═O)Me;
-Me, -Et, -nPr, -iPr;
phenyl, pyrrolyl, thienyl, furyl, imidazolyl, oxazolyl, isoxazolyl, thiazolyl, isothiazolyl, pyrazolyl, pyridyl, pyrazinyl, pyrimidinyl, pyridazinyl, benzyl, phenylethyl, pyridyl-methyl, isothiazolyl-methyl, each optionally substituted, for example, with one or more of —F, —Cl, —Br, —I, —OH, —OMe, —OEt, —O(nPr), —O(iPr), —NH₂, —NHMe, —NHEt, —NH(nPr), —NH(iPr), —NMe₂, —NEt₂, —N(nPr)₂, —N(iPr)₂, pyrrolidino, piperidino, piperazino, morpholino, —COOH, —COMe, —COEt, —CO(nPr), —CO(iPr), —CONH₂, —CONHMe, —CONHEt, —CONH(nPr), —CONH(iPr), —CONMe₂, —CONEt₂, —CON(nPr)₂, —CON(iPr)₂, -Me, -Et, -nPr, -iPr;
—(CH₂)ₘ—OH;
—(CH₂)ₘ—OMe, —(CH₂)ₘ—OEt, —(CH₂)ₘ—O(nPr), —(CH₂)ₘ—O(iPr);
—(CH₂)ₘ—COOH;
—(CH₂)ₘ—COOMe, —(CH₂)ₘ—COOEt, —(CH₂)ₘ—COO(nPr), —(CH₂)ₘ—COO(iPr);
—(CH₂)ₘ—NH₂;
—(CH₂)ₘ—NH Me, —(CH₂)ₘ—NMe₂, —(CH₂)ₘ—NHEt, —(CH₂)ₘ—NEt₂, —(CH₂)ₘ—NH(nPr), —(CH₂)ₘ—N(nPr)₂, —(CH₂)ₘ—NH(iPr), —(CH₂)ₘ—N(iPr)₂, —(CH₂)ₘ-pyrrolidino, —(CH₂)ₘ-piperidino, —(CH₂)ₘ-piperazino, —(CH₂)ₘ-morpholino;
—(CH₂)ₘ—CONH Me, —(CH₂)ₘ—CONMe₂, —(CH₂)ₘ—CONH Et, —(CH₂)ₘ—CONEt₂, —(CH₂)ₘ—CONH(nPr), —(CH₂)ₘ—CON(nPr)₂, —(CH₂)ₘ—CONH(iPr), —(CH₂)ₘ—CON(iPr)₂, —(CH₂)ₘ—CO-pyrrolidino, —(CH₂)ₘ—CO-piperidino, —(CH₂)ₘ—CO-piperazino, —(CH₂)ₘ—CO-morpholino;
—O—(CH₂)ₘ—OH;
—O—(CH₂)ₘ—OMe, —O—(CH₂)ₘ—OEt, —O—(CH₂)ₘ—O(nPr), —O—(CH₂)ₘ—O(iPr);
—O—(CH₂)ₘ—COOH;
—O—(CH₂)ₘ—COOMe, —O—(CH₂)ₘ—COOEt, —O—(CH₂)ₘ—COO(nPr), —O—(CH₂)ₘ—COO(iPr);
—O—(CH₂)ₘ—NH₂;
—O—(CH₂)ₘ—NHMe, —O—(CH₂)ₘ—NMe₂, —O—(CH₂)ₘ—NHEt, —O—(CH₂)ₘ, —NEt₂, —O—(CH₂)ₘ—NH(nPr), —O—(CH₂)ₘ—N(nPr)₂, —O—(CH₂)ₘ—NH(iPr), —O—(CH₂)ₘ—N(iPr)₂, —O—(CH₂)ₘ-pyrrolidino, —O—(CH₂)ₘ-piperidino, —O—(CH₂)ₘ-piperazino, —O—(CH₂)ₘ-morpholino;
—O—(CH₂)ₘ—CONHMe, —O—(CH₂)ₘ—CONMe₂, —O—(CH₂)ₘ—CONHEt, —O—(CH₂)ₘ—CONEt₂, —O—(CH₂)ₘ—CONH(nPr), —O—(CH₂)ₘ—CON(nPr)₂, —O—(CH₂)ₘ—CONH(iPr), —O—(CH₂)ₘ—CON(iPr)₂, —O—(CH₂)ₘ—CO-pyrrolidino, —O—(CH₂)ₘ—CO-piperidino, —O—(CH₂)ₘ—CO-piperazino, —O—(CH₂)ₘ—CO-morpholino;
—NH—(CH₂)ₘ—OH;
—NH—(CH₂)ₘ—OMe, —NH—(CH₂)ₘ—OEt, —NH—(CH₂)ₘ—O(nPr), —NH—(CH₂)ₘ—O(iPr);
—NH—(CH₂)ₘ—COOH;
—NH—(CH₂)ₘ—COOMe, —NH—(CH₂)ₘ—COOEt, —NH—(CH₂)ₘ—COO(nPr), —NH—(CH₂)ₘ—COO(iPr);
—NH—(CH₂)ₘ—NH₂;
—NH—(CH₂)ₘ—NHMe, —NH—(CH₂)ₘ—NMe₂, —NH—(CH₂)ₘ—NHEt, —NH—(CH₂)ₘ—NEt₂, —NH—(CH₂)ₘ—NH(nPr), —NH—(CH₂)ₘ—N(nPr)₂, —NH—(CH₂)ₘ—NH(iPr), —NH—(CH₂)ₘ—N(iPr)₂, —NH—(CH₂)ₘ-pyrrolidino, —NH—(CH₂)ₘ-piperidino, —NH—(CH₂)ₘ-piperazino, —NH—(CH₂)ₘ-morpholino;
—NH—(CH₂)ₘ—CONHMe, —NH—(CH₂)ₘ—CONMe₂, —NH—(CH₂)ₘ—CONHEt, —NH—(CH₂)ₘ—CONEt₂, —NH—(CH₂)ₘ—CONH(nPr), —NH—(CH₂)ₘ—CON(nPr)₂, —NH—(CH₂)ₘ—CONH(iPr), —NH—(CH₂)ₘ—CON(iPr)₂, —NH—(CH₂)ₘ—CO-pyrrolidino, —NH—(CH₂)ₘ—CO-piperidino, —NH—(CH₂)ₘ—CO-piperazino, —NH—(CH₂)ₘ—CO-morpholino;
wherein each m is independently 1, 2, 3, or 4.

In one embodiment, each monovalent monodentate substituent is independently selected from:
—H;
—F; —Cl; —Br; —I;
—OH;
—OMe, —OEt, —O(nPr), —O(iPr);
—C(═O)OH;
—C(═O)OMe, —C(═O)OEt, —C(═O)O(nPr), —C(═O)O(iPr);
—C(═O)NH₂;
—C(═O)NHMe, —C(═O)NHEt, —C(═O)NH(nPr), —C(═O)NH(iPr);
—C(═O)NMe₂, —C(═O)NEt₂, —C(═O)N(nPr)₂, —C(═O)N(iPr)₂;
—C(═O)-pyrrolidino, —C(═O)-piperidino, —C(═O)-piperazino, —C(═O)-morpholino;
—NH₂;
—NHMe, —NHEt, —NH(nPr), —NH(iPr);
—NMe₂, —NEt₂, —N(nPr)₂, —N(iPr)₂;
pyrrolidino, piperidino, piperazino, morpholino;
—NHC(═O)H;
—NMeC(═O)H, —NEtC(═O)H, —N(nPr)C(═O)H, —N(iPr)C(═O)H;
—NHC(═O)Me, —NHC(═O)Et, —NHC(═O)(nPr), —NHC(═O)(iPr);
-Me, -Et, -nPr, -iPr;
phenyl, pyridyl, , benzyl, phenylethyl, pyridyl-methyl, each optionally substituted, for example, with one or more of —F, —Cl, —Br, —I, —OH, —OMe, —OEt, —O(nPr), —O(iPr), —NH₂, —NHMe, —NHEt, —NH(nPr), —NH(iPr), —NMe₂, —NEt₂, —N(nPr)₂, —N(iPr)₂, pyrrolidino, piperidino, piperazino, morpholino, —COOH, —CONH₂, —CONHMe, —CONHEt, —CONH(nPr), —CONH(iPr), -Me, -Et, -nPr, -iPr;
—(CH₂)ₘ—OH;
—(CH₂)ₘ—OMe, —(CH₂)ₘ—OEt, —(CH₂)ₘ—O(nPr), —(CH₂)ₘ—O(iPr);
—(CH₂)ₘ—COOH;
—(CH₂)ₘ—COOMe, —(CH₂)ₘ—COOEt, —(CH₂)ₘ—COO(nPr), —(CH₂)ₘ—COO(i Pr);
—(CH₂)ₘ—NH₂;
—(CH₂)ₘ—NH Me, —(CH₂)ₘ—NMe₂, —(CH₂)ₘ—NHEt, —(CH₂)ₘ—NEt₂, —(CH₂)ₘ—NH(nPr), —(CH₂)ₘ—N(nPr)₂, —(CH₂)ₘ—NH(iPr), —(CH₂)ₘ—N(iPr)₂, —(CH₂)ₘ-pyrrolidino, —(CH₂)ₘ-piperidino, —(CH₂)ₘ-piperazino, —(CH₂)ₘ-morpholino;
—(CH₂)ₘ—CONHMe, —(CH₂)ₘ—CONMe₂, —(CH₂)ₘ—CONHEt, —(CH₂)ₘ—CONEt₂, 15-(CH₂)ₘ—CONH(nPr), —(CH₂)ₘ—CON(nPr)₂, —(CH₂)ₘ—CONH(iPr);
—O—(CH₂)ₘ—OH;
—O—(CH₂)ₘ—OMe, —O—(CH₂)ₘ—OEt, —O—(CH₂)ₘ—O(nPr), —O—(CH₂)ₘ—O(iPr);
—O—(CH₂)ₘ—COOH;
—O—(CH₂)ₘ—COOMe, —O—(CH₂)ₘ—COOEt, —O—(CH₂)ₘ—COO(nPr), —O—(CH₂)ₘ—COO(iPr);
—O—(CH₂)ₘ—NH₂;
—O—(CH₂)ₘ—NHMe, —O—(CH₂)ₘ—NMe₂, —O—(CH₂)ₘ—NHEt, —O—(CH₂)ₘ—NEt₂, —O—(CH₂)ₘ—NH(nPr), —O—(CH₂)ₘ—N(nPr)₂, —O—(CH₂)ₘ—NH(iPr), —O—(CH₂)ₘ—N(iPr)₂, —O—(CH₂)ₘ-pyrrolidino, —O—(CH₂)ₘ-piperidino, —O—(CH₂)ₘ-piperazino, —O—(CH₂)ₘ-morpholino;
—O—(CH₂)ₘ—CONH Me, —O—(CH₂)ₘ—CONMe₂, —O—(CH₂)ₘ—CONHEt, —O—(CH₂)ₘ—CONEt₂, —O—(CH₂)ₘ—CONH(nPr), —O—(CH₂)ₘ—CON(nPr)₂, —O—(CH₂)ₘ—CONH(iPr);
—NH—(CH₂)ₘ—OH;
—NH—(CH₂)ₘ—OMe;
—NH—(CH₂)ₘ—NH₂; —NH—(CH₂)ₘ—NHMe, —NH—(CH₂)ₘ—NMe₂, —NH—(CH₂)ₘ—NHEt, —NH—(CH₂)ₘ—NEt₂, —NH—(CH₂)ₘ—NH(nPr), —NH—(CH₂)ₘ—N(nPr)₂, —NH—(CH₂)ₘ—NH(iPr), —NH—(CH₂)ₘ—N(iPr)₂, —NH—(CH₂)ₘ-pyrrolidino, —NH—(CH₂)ₘ-piperidino, —NH—(CH₂)ₘ-piperazino, —NH—(CH₂)ₘ-morpholino;
—NH—(CH₂)ₘ—CONH Me, —NH—(CH₂)ₘ—CONMe₂, —NH—(CH₂)ₘ—CONHEt, —NH—(CH₂)ₘ—CONEt₂, —NH—(CH₂)ₘ—CONH(nPr), —NH—(CH₂)ₘ—CON(nPr)₂, —NH—(CH₂)ₘ—CONH(iPr), —NH—(CH₂)ₘ—CON(iPr)₂, —NH—(CH₂)ₘ—CO-pyrrolidino, —NH—(CH₂)ₘ—CO-piperidino, —NH—(CH₂)ₘ—CO-piperazino, —NH—(CH₂)ₘ—CO-morpholino;
wherein each m is independently 1, 2, or 3.

In one embodiment, each monovalent monodentate substituent is independently selected from:
—H;
—F, —Cl;

—OH;
—OMe, —OEt,
—C(═O)OH;
—C(═O)OMe, —C(═O)OEt;
—C(═O)NH₂;
—C(═O)NHMe, —C(═O)NHEt;
—NH₂;
—NHMe, —NHEt, —NH(iPr);
—NMe₂, —NEt₂;
pyrrolidino, piperidino, piperazino, morpholino;
-Me, -Et, -nPr, -iPr;
phenyl, pyridyl, each optionally substituted, for example, with one or more of —F, —Cl, —OH, —OMe, —OEt, —COOH, —CONH₂, —CONHMe, —CONHEt, -Me, -Et, -iPr;
—(CH₂)ₘ—OH;
—(CH₂)ₘ—OMe;
—(CH₂)ₘ—NH₂;
—(CH₂)ₘ—NH Me, —(CH₂)ₘ—NMe₂, —(CH₂)ₘ—NH Et, —(CH₂)ₘ—NEt₂, —(CH₂)ₘ-pyrrolidino, —(CH₂)ₘ-piperidino, —(CH₂)ₘ-piperazino, —(CH₂)ₘ-morpholino;
—(CH₂)ₘ—CONHMe, —(CH₂)ₘ—CONHEt;
—O—(CH₂)ₘ—OH;
—O—(CH₂)ₘ—NH₂;
—O—(CH₂)ₘ—NMe₂, —O—(CH₂)ₘ—NEt₂, —O—(CH₂)ₘ-morpholino;
wherein each m is independently 1, 2, or 3.

In one embodiment, each monovalent monodentate substituent is independently selected from:
—H;
—Cl;
—OH;
—OMe;
—C(═O)NH₂;
—C(═O)NHMe, —C(═O)NHEt;
-Me, -Et, -iPr;
phenyl, pyridyl, each optionally substituted, for example, with one or more of —F, —Cl, —OH, —OMe, —CONH₂, —CONHMe, —CONHEt, -Me, -Et, and -iPr;
—(CH₂)ₘ—OH;
—(CH₂)ₘ—OMe;
—(CH₂)ₘ—NH₂;
—(CH₂)ₘ—NMe₂, —(CH₂)ₘ—NEt₂, —(CH₂)ₘ-morpholino;
—(CH₂)ₘ—CONHMe, —(CH₂)ₘ—CONHEt;
—O—(CH₂)ₘ—OH;
—O—(CH₂)ₘ—NH₂;
—O—(CH₂)ₘ—NMe₂, —O—(CH₂)ₘ—NEt₂, —O—(CH₂)ₘ-morpholino;
wherein each m is independently 1, 2, or 3.

In one embodiment, each monovalent monodentate substituent on the A-ring and B-ring is independently selected from:
—H;
unsubstituted aliphatic C₁₋₆alkyl (e.g., -Me, -Et, -iPr);
optionally substituted C₆₋₁₀carboaryl (e.g., phenyl); and
optionally substituted C₅₋₁₀heteroaryl;
   wherein the optional substituents on C₆₋₁₀carboaryl and C₅₋₁₀heteroaryl are selected from:
halogen (e.g., —F, —Cl), —R^{t}, OR^{t}, —NH(R^{t}), —N(R^{t})₂, —CH₂NH(R^{t}), —CH₂N(R^{t})₂, —CONHR^{t}, —O(CH₂)ₜNR^{t}, —CH₂CONHR^{t}, and —CH₂CON(R^{t})₂;
   wherein:
   each R^{t} is independently unsubstituted aliphatic C₁₋₆alkyl (e.g., -Me, -Et, or -iPr), and each t is independently 1, 2, 3, 4, 5, or 6 (e.g., 2 or 3).

In one embodiment, each monovalent monodentate substituent on each of the C-ring, D-ring, E-ring, and G-ring, if present, is independently selected from:
—H, —R^{t}, —OR^{t}, —NH(R^{t}), —N(R^{t})₂, —CH₂NH(R^{t}), —CH₂N(R^{t})₂, —CONHR^{t}, —O(CH₂)ₜNR^{t}, —CH₂CONHR^{t}, and —CH₂CON(Rt)₂;
   wherein:
   each R^{t} is independently unsubstituted aliphatic C₁₋₆alkyl, and
   each t is independently 1, 2, 3, 4, 5, or 6.

In one embodiment, each Rt is independently -Me, -Et, or -iPr.

In one embodiment, each t is independently 2 or 3.

In one embodiment, each monovalent monodentate substituent on the F-ring, if present, is independently selected from:
—H;
unsubstituted aliphatic C₁₋₆alkyl (e.g., -Me, -Et, -iPr);
halogen (e.g., —F, —Cl);
optionally substituted C₆₋₁₀carboaryl (e.g., phenyl); and
optionally substituted C₅₋₁₀heteroaryl;
   wherein the optional substituents on C₆₋₁₀carboaryl and C₅₋₅₀heteroaryl are selected from:
halogen (e.g., —F, —Cl), —R^{t}, —ORt, —NH(R^{t}), —N(R^{t})₂, —CH₂NH(R^{t}), —CH₂N(R^{t})₂, —CONHR^{t}, —O(CH₂)ₜNR^{t}, —CH₂CONHR^{t}, and —CH₂CON(R^{t})₂;
   wherein:
   each R^{t} is independently unsubstituted aliphatic C₁₋₆alkyl (e.g., -Me, -Et, or -iPr), and each t is independently 1, 2, 3, 4, 5, or 6 (e.g., 2 or 3).

### The Ring Nitrogen Substituent R^{NA}

In one embodiment, the A-ring nitrogen substituent, R^{NA}, if present, is independently selected from:

### (1) —H;

### (11) —R;

### (12) —R^{Alk}—OH; —R^{Alk}—OR

### (13) —R^{Alk}—NH₂; —R^{Alk}—NH R; —R^{Alk}—NR₂; —R^{Alk}—NR^{N1}R^{N2};

### (14) —R^{Alk}—C(═O)OH; —R^{Alk}—C(═O)OR;

(15) —R^{Alk}—C(═O)NH₂; —R^{Alk}—C(═O)NH R; —R^{Alk}—C(═O)NR₂; —R^{Alk}—C(═O)NR^{N1}R^{N2};
wherein each R, R^{Alk}, R^{N1}, and R^{N2} is as defined above under the heading “The Monovalent Mondentate Substituents”.

In one embodiment, the A-ring nitrogen substituent, R^{NA}, if present, is independently selected from:
—H;
-Me, -Et, -nPr, -iPr;
—(CH₂)ₘ—OH;
—(CH₂)ₘ, —OMe, —(CH₂)ₘ—OEt, —(CH₂)ₘ—O(nPr), —(CH₂)ₘ—O(iPr);
—(CH₂)ₘ—COOH;
—(CH₂)ₘ—COOMe, —(CH₂)ₘ—COOEt, —(CH₂)ₘ—COO(nPr), —(CH₂)ₘ—COO(i Pr);
—(CH₂)ₘ—NH₂;
—(CH₂)ₘ—NH Me, —(CH₂)ₘ—NMe₂, —(CH₂)ₘ—NHEt, —(CH₂)ₘ—NEt₂, —(CH₂)ₘ—NH(nPr), —(CH₂)ₘ—N(nPr)₂, —(CH₂)ₘ—NH(iPr), —(CH₂)ₘ—N(iPr)₂, —(CH₂)ₘ-pyrrolidino, —(CH₂)ₘ-piperidino, —(CH₂)ₘ-piperazino, —(CH₂)ₘ-morpholino;
—(CH₂)ₘ—CONHMe, —(CH₂)ₘ—CONMe₂, —(CH₂)ₘ—CONHEt, —(CH₂)ₘ—CONEt₂, —(CH₂)ₘ—CONH(nPr), —(CH₂)ₘ—CON(nPr)₂, —(CH₂)ₘ—CONH(iPr), —(CH₂)ₘ—CON(iPr)₂, —(CH₂)ₘ—CO-pyrrolidino, —(CH₂)ₘ—CO-piperidino, —(CH₂)ₘ—CO-piperazino, —(CH₂)ₘ—CO-morpholino;
wherein each m is independently 1, 2, 3, or 4.

In one embodiment, each monovalent monodentate substituent is independently selected from:

### —H;

-Me, -Et, -iPr;
—(CH₂)ₘ—OH;
—(CH₂)ₘ—NH₂;
—(CH₂)ₘ—NMe₂, —(CH₂)ₘ—NEt₂, and —(CH₂)ₘ-morpholino;
wherein each m is independently 2 or 3.

In one embodiment, the A-ring nitrogen substituent, R^{NA}, if present, is independently selected from: —H, -Me, and -Et.

In one embodiment, the A-ring nitrogen substituent, R^{NA}, if present, is -Et.

In one embodiment, the A-ring nitrogen substituent, R^{NA}, if present, is -Me.

In one embodiment, the A-ring nitrogen substituent, R^{NA}, if present, is —H.

### The Ring Nitrogen Substituent R^{NB}

In one embodiment, the B-ring nitrogen substituent, R^{NB}, is independently as defined above for R^{NA}.

### Molecular Weight

In one embodiment, the compound has a molecular weight of 200 to 1000.

In one embodiment, the bottom of range is 225; 250; 275; 300; 325; 350; 375; 400; 425; 450.

In one embodiment, the top of range is 900; 800; 700; 600; 500.

In one embodiment, the range is 200 to 900.

In one embodiment, the range is 200 to 800.

In one embodiment, the range is 200 to 700.

In one embodiment, the range is 200 to 600.

In one embodiment, the range is 200 to 500.

### Combinations

Each and every compatible combination of the embodiments described above is explicitly disclosed herein, as if each and every combination was individually and explicitly recited.

### CERTAIN PREFERRED EMBODIMENTS

In one embodiment, the compounds are selected from compounds of the following formulae:

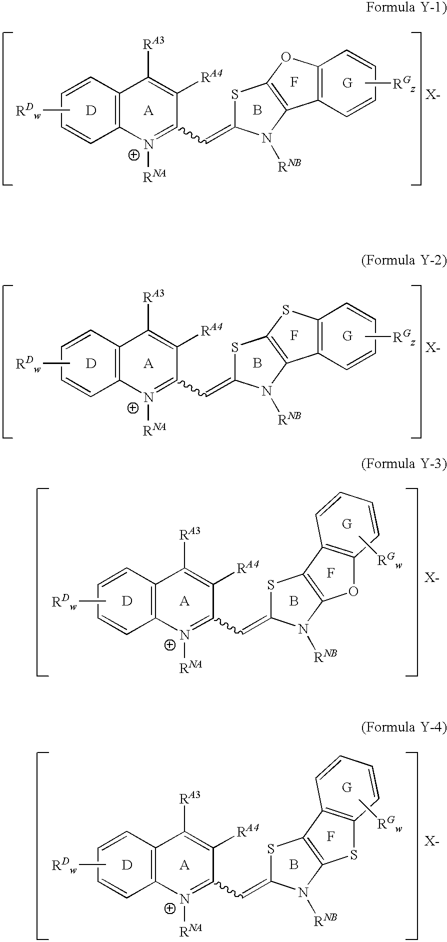

wherein:
R^{NA} and X⁻ may be present, or R^{NA} and X⁻ may be absent;
if R^{NA} and X⁻ are present, then:
   the nitrogen atom of the A-ring bears a positive charge;
if R^{NA} and X⁻ are absent, then:
   the nitrogen atom of the A-ring is electrically neutral;
      and wherein:
each of R^{A3} and R^{A4} is independently an A-ring monovalent monodentate substituent;
w is 0, 1, 2, 3, or 4, and each R^{D}, if present, is independently a D-ring substituent;
z is 0, 1, 2, 3, or 4, and each R^{G}, if present, is independently a G-ring substituent;
   and wherein:
R^{NA}, if present, is independently an A-ring nitrogen substituent;
R^{NB} is independently a B-ring nitrogen substituent;
the linkage joining the A-ring and the B-ring is cis or trans;
and pharmaceutically acceptable salts, solvates, amides, esters, ethers, N-oxides, chemically protected forms, and prodrugs thereof.

In one embodiment, the compounds are selected from compounds of the following formulae:

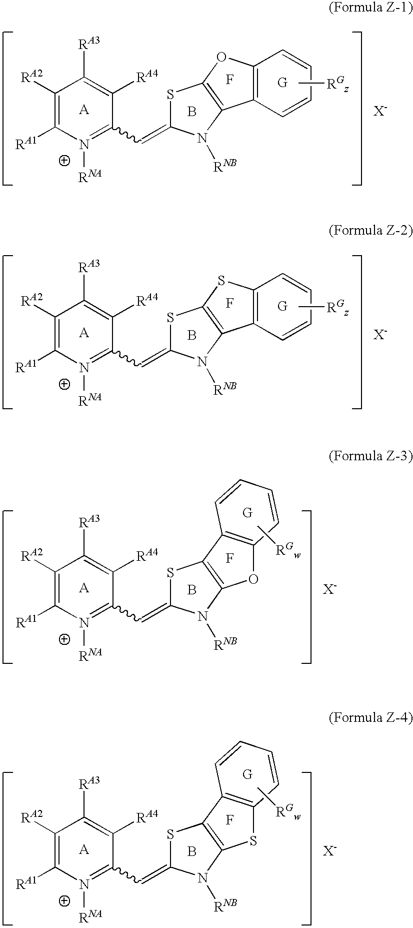

wherein:
R^{NA} and X⁻ may be present, or R^{NA} and X⁻ may be absent;
if R^{NA} and X⁻ are present, then:
   the nitrogen atom of the A-ring bears a positive charge;
if R^{NA} and X⁻ are absent, then:
   the nitrogen atom of the A-ring is electrically neutral;
      and wherein:
each of R^{A1}, R^{A2}, R^{A3}, and R^{A4} is independently an A-ring monovalent monodentate substituent;
z is 0, 1, 2, 3, or 4, and each R^{G}, if present, is independently a G-ring substituent;
   and wherein:
R^{NA}, if present, is independently an A-ring nitrogen substituent;
R^{NB} is independently a B-ring nitrogen substituent;
the linkage joining the A-ring and the B-ring is cis or trans;
and pharmaceutically acceptable salts, solvates, amides, esters, ethers, N-oxides, chemically protected forms, and prodrugs thereof.

### SOME PREFERRED EMBODIMENTS

Some preferred examples of the compounds described above include the following:

| # | Structure | Compound |
|---|---|---|
| 1. | 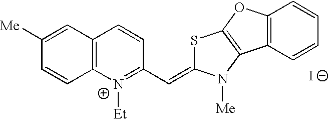 | MMR201 |
| 2. | 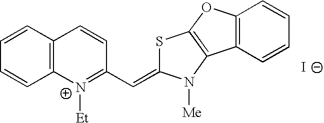 | MMR211 |
| 3. | 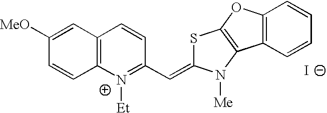 | MMR216 |

Some preferred examples of the compounds described above include the following:

| # | Structure | Compound |
|---|---|---|
| 4. | 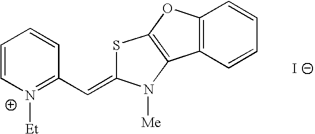 | MMR307 |

Some preferred examples of the compounds described above include the following:

| | | |
|---|---|---|
| 5. | 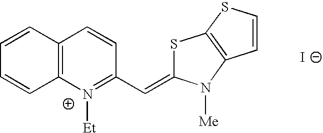 | MMR218 |

Some preferred examples of the compounds described above include the following:

| # | Structure | Compound |
|---|---|---|
| 6. | 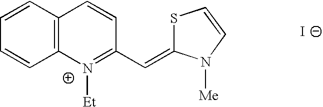 | MMR212 |
| 7. | 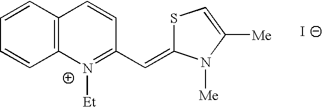 | MMR213 |
| 8. | 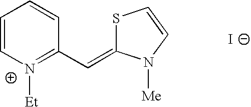 | MMR302 |
| 9. | 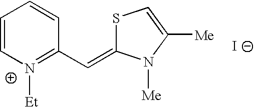 | MMR304 |
| 10. | 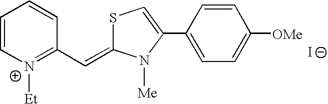 | MMR308 |
| 11. | 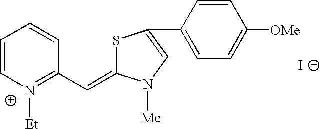 | MMR309 |

In one embodiment, the compound is selected from the above compounds, and pharmaceutically acceptable salts, solvates, amides, esters, ethers, N-oxides, chemically protected forms, and prodrugs thereof.

### Additional Compounds

Another aspect of the present invention pertains to the compounds described below, which are not encompassed by the above definitions, because the ring that would correspond to the F-ring above is a 6 membered ring. (The F-ring, if present, is a 5-membered heteroaromatic ring, and is NOT a 6-membered ring.)

Thus, other aspects of the present invention pertains to the following compounds, and pharmaceutically acceptable salts, solvates, amides, esters, ethers, N-oxides, chemically protected forms, and prodrugs thereof; compositions comprising them, as described herein; and their use, as described herein.

| # | Structure | Compound |
|---|---|---|
| 12. | 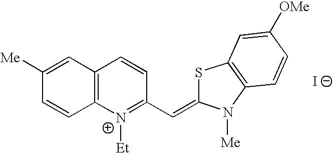 | MMR202 |
| 13. | 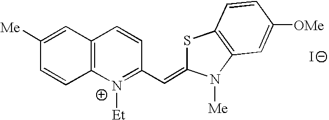 | MMR203 |
| 14. | 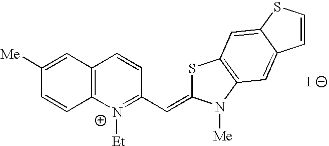 | MMR204 |
| 15. | 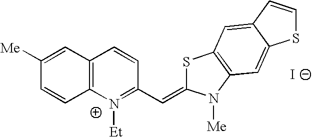 | MMR205 |
| 16. | 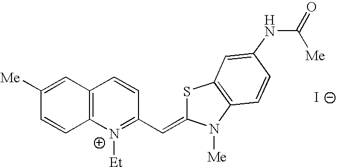 | MMR206 |
| 17. | 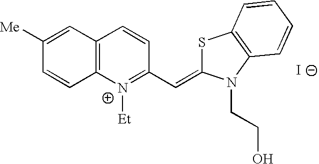 | MMR207 |
| 18. | 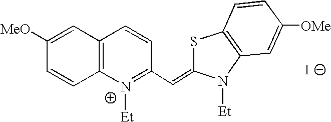 | MMR208 |
| 19. | 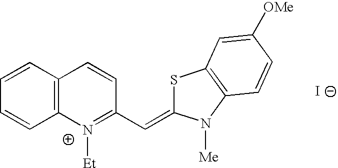 | MMR209 |
| 20. | 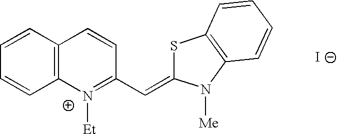 | MMR210 |
| 21. | 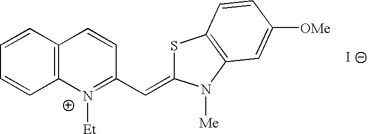 | MMR214 |
| 22. | 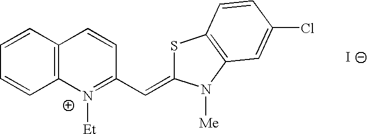 | MMR215 |
| 23. | 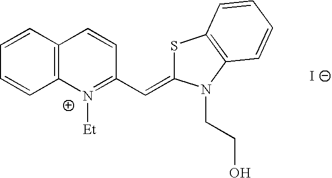 | MMR217 |
| 24. | 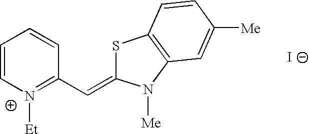 | MMR301 |
| 25. | 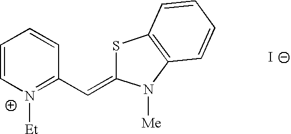 | MMR303 |
| 26. | 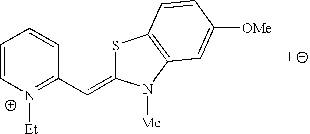 | MMR305 |
| 27. | 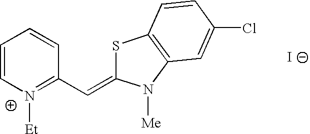 | MMR306 |
| 28. | 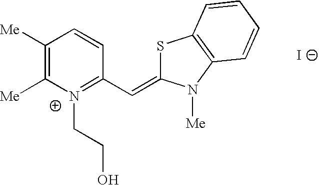 | MMR310 |
| 29. | 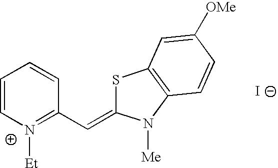 | MMR311 |
| 30. | 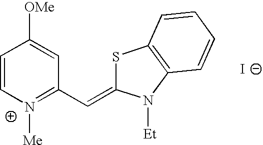 | MMR312 |

### Isomers

Certain compounds may exist in one or more particular geometric, optical, enantiomeric, diastereomeric, epimeric, atropic, stereoisomeric, tautomeric, conformational, or anomeric forms, including but not limited to, cis- and trans-forms; E- and Z-forms; c-, t-, and r-forms; endo- and exo-forms; R—, S—, and meso-forms; D- and L-forms; d- and 1-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

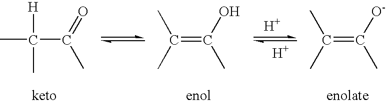

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including 160 and 180; and the like.

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### Salts

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, trifluoroacetic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Solvates

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes solvate forms thereof.

### Chemically Protected Forms

It may be convenient or desirable to prepare, purify, and/or handle the active compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999).

Unless otherwise specified, a reference to a particular compound also includes chemically protected forms thereof.

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl(diphenylmethyl), or trityl(triphenylmethyl)ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases (e.g., cyclic amines), as a nitroxide radical (>N—O.).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇-trihaloalkyl ester); a triC₁₋₇alkylsilyl-C₁₋₇alkyl ester; or a C₅₋₂₀aryl-C₁₋₁₇alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

### Prodrugs

It may be convenient or desirable to prepare, purify, and/or handle the active compound in the form of a prodrug. The term “prodrug,” as used herein, pertains to a compound which, when metabolised (e.g., in vivo), yields the desired active compound. Typically, the prodrug is inactive, or less active than the active compound, but may provide advantageous handling, administration, or metabolic properties.

Unless otherwise specified, a reference to a particular compound also includes prodrugs thereof.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

### Chemical Synthesis

Several methods for the chemical synthesis of compounds of the present invention are described herein. These and/or other well-known methods may be modified and/or adapted in known ways in order to facilitate the synthesis of additional compounds of the present invention. Additionally, several of the compounds described herein may be obtained from commercial sources.

In one approach, target compounds (1) may be prepared by a method that involves the base promoted condensation reaction between a 2-iodopyridinium alkiodide (2) (or a 2-iodoquinolinium alkiodide) and a 2-methylthiazolium salt (3), for example, in analogy to the methods described in Hamer et al., 1928.

The 2-iodopyridinium alkiodide (2) (or 2-iodoquinolinium alkiodide) may itself be prepared by reaction of the corresponding 2-chloropyridine or 2-bromopyridine (4) (or 2-chloroquinoline or 2-bromoquinoline) with a suitable alkyl iodide (5), for example, in analogy to the methods described in Hamer et al., 1928.

The 2-methylthiazolium salt (3) may be prepared from the corresponding 2-methylthiazole (8) by reaction with a suitable alkylating agent (9), for example, in analogy to the methods described in Hojo et al., 1988.

Where the 2-methylthiazole (8) is not available from commercial sources, it can be synthesized using known methods, for example, the well-known (“Hantzsch Synthesis”) reaction of the corresponding 2-bromoketone (6) with thioacetamide (7), for example, in analogy to the methods described in Joule et al., 1995.

An example of such an approach is illustrated in the following scheme.

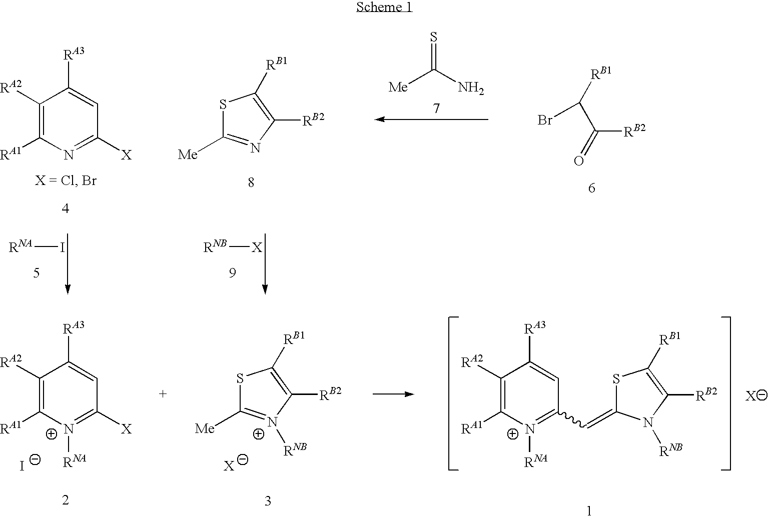

In a related approach, target compounds (13) containing a benzofuranothiazole tricycle component can be prepared using a base promoted condensation reaction between 2-iodopyridinium alkiodide (2) (or a 2-iodoquinolinium alkiodide) and a 2-methylbenzofuranothiazolium salt (12), in analogy to the condensation of a 2-iodopyridinium alkiodide (2) (or 2-iodoquinolinium alkiodide) and a 2-methylthiazolium salt (3), as described above.

The 2-methyl-benzofuranothiazolium salt (12) can be prepared by alkylation of a 2-methyl-benzofuranothiazole (11), which can itself be prepared by the reaction of the 2-bromobenzofuran-3-(2H)-one (10) with thioacetamide (7), for example, in analogy to the methods described in Bogolyubskaya et al., 1964.

An example of such an approach is illustrated in the following scheme.

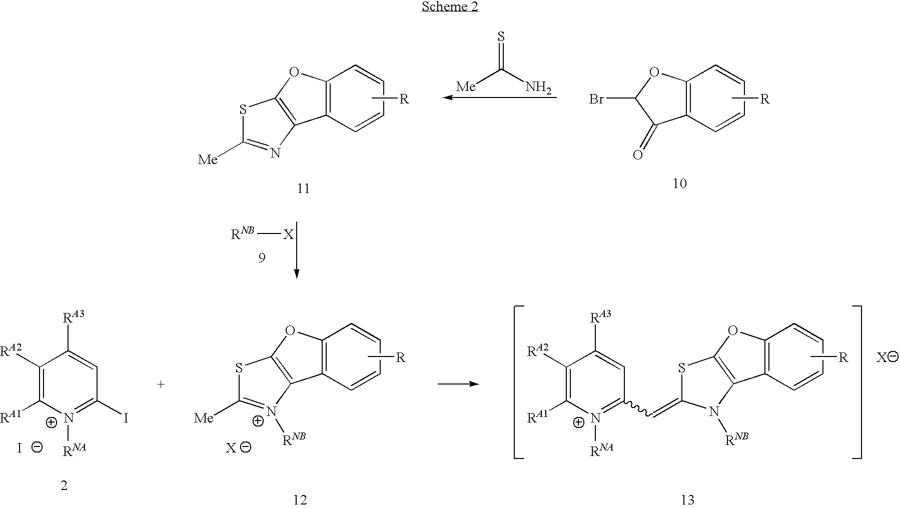

In another approach, target compounds (1) may be prepared by a method that involves the base-promoted condensation reaction of a 2-methylpyridinium salt (15) (or a quinolinium salt) with a 2-(methylthio)thiazolium salt (19), for example, in analogy to the methods described in Hamer et al., 1964.

The 2-methylpyridinium salt (15) (or a quinolinium salt) may be prepared by reaction of the corresponding 2-methylpyridine (14) (or 2-methylquinoline) with a suitable alkylating agent (9). The thiazolium salt (19) can be prepared from the corresponding 2-(methylthio)thiazole (18) using a suitable alkylating agent (9), for example, in analogy to the methods described in Kawakami et al., 1997.

Where the 2-(methylthio)thiazole (18) is not available from commercial sources, it can be synthesized using known methods, for example, the well known reaction of a corresponding 2-bromoketone (6) with ammonium dithiocarbamate (16), for example, in analogy to the methods described in Buchman et al., 1941, followed by reaction with a suitable methylating agent (17), for example, in analogy to the methods described in Frey et al., 2003.

An example of such an approach is illustrated in the following scheme.

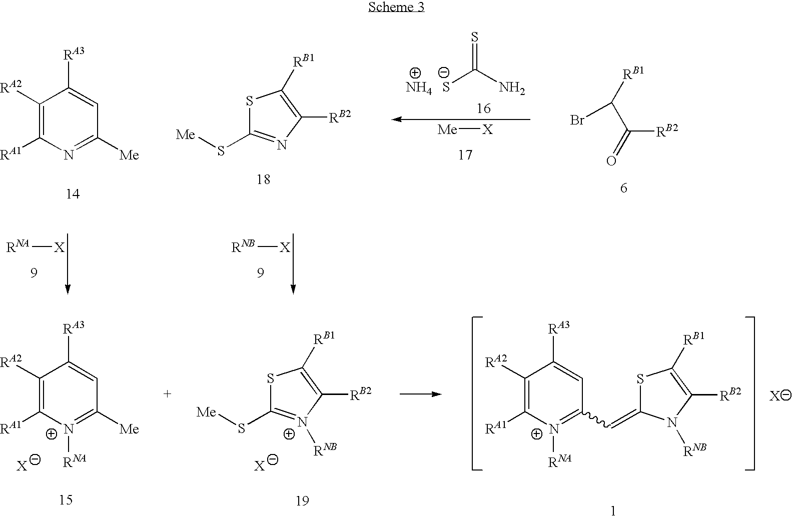

In a related approach, target compounds (13) containing a benzofuranothiazole tricycle component can be prepared using a base promoted condensation reaction between a 2-methylpyridinium salt (15) (or a 2-methylquinolinium salt) and a 2-(methylthio) benzofuranothiazolium salt (21), in analogy to the condensation of a 2-methylpyridinium salt (15) with a 2-(methylthio)thiazolium salt (19), as described above.

The thiazolium salt (21) can itself be prepared by alkylation of a 2-(methylthio)benzofuranothiazole (20), which can itself be prepared by the reaction of a 2-bromobenzofuran-3-(2H)-one (10) with ammonium dithiocarbamate (16), followed by reaction with a suitable methylating agent (17).

An example of such an approach is illustrated in the following scheme.

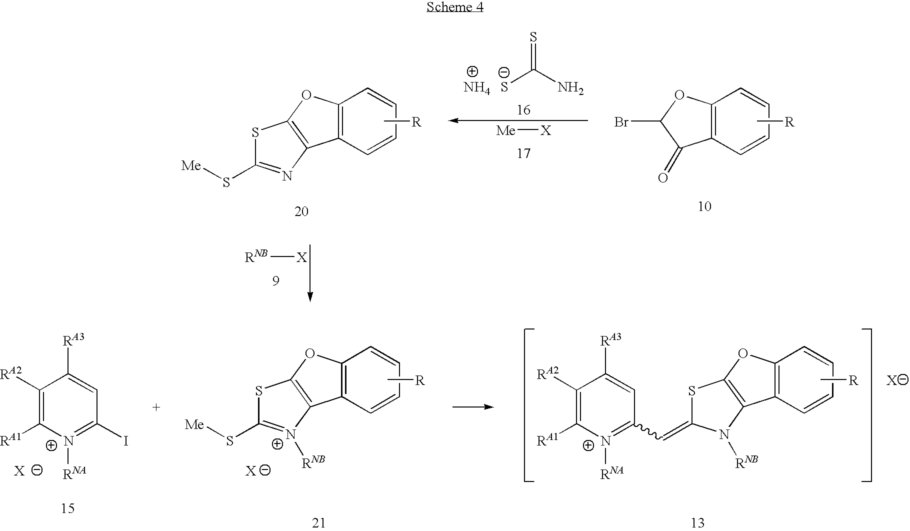

In another approach, useful intermediates are prepared by reacting a suitable substituted aniline (22) with a cinnamoyl halide (23) to form the corresponding amide (24), which is then reacted with AlCl₃ to close the ring and form a quinolinone (25), which is then reacted with POCl₃ to form the corresponding chloroquinoline (26), which is then reacted with ethyl iodide to form the corresponding N-ethyl iodo compound (27), which can be used in the synthesis of the compounds described herein. See, for example, Inglis et al., 2004.

An example of such an approach is illustrated in the following scheme.

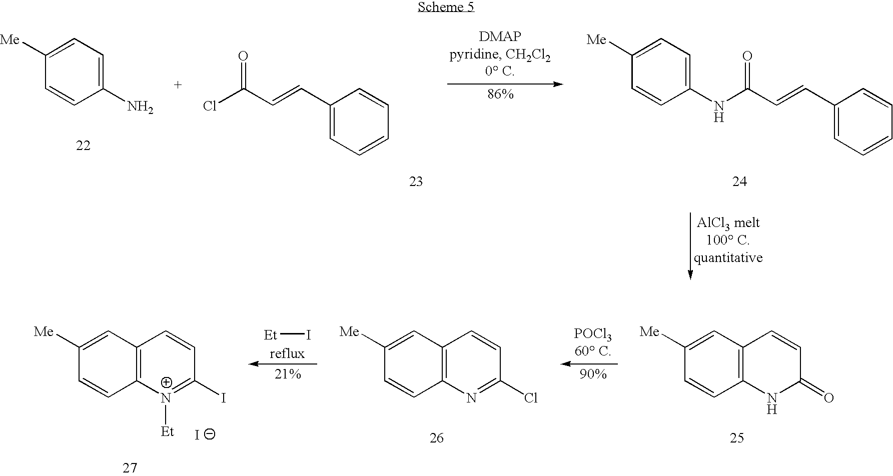

In a related approach, target compounds (31) containing a benzothiofuranothiazole tricycle component can be prepared using a base promoted condensation reaction between 2-iodopyridinium alkiodide (2) (or a 2-iodoquinolinium alkiodide) and a 2-methyl-benzothiofuranothiazolium salt (30), in analogy to the condensation of a 2-iodopyridinium alkiodide (2) (or 2-iodoquinolinium alkiodide) and a 2-methylthiazolium salt (3), as described above.

The 2-methyl-benzothiofuranothiazolium salt (30) can be prepared by alkylation of a 2-methyl-benzothiofuranothiazole (29), which can itself be prepared by the reaction of the 2-bromobenzothiofuran-3-(2H)-one (28) with thioacetamide, for example, in analogy to the methods described in Bogolyubskaya et al., 1964.

An example of such an approach is illustrated in the following scheme.

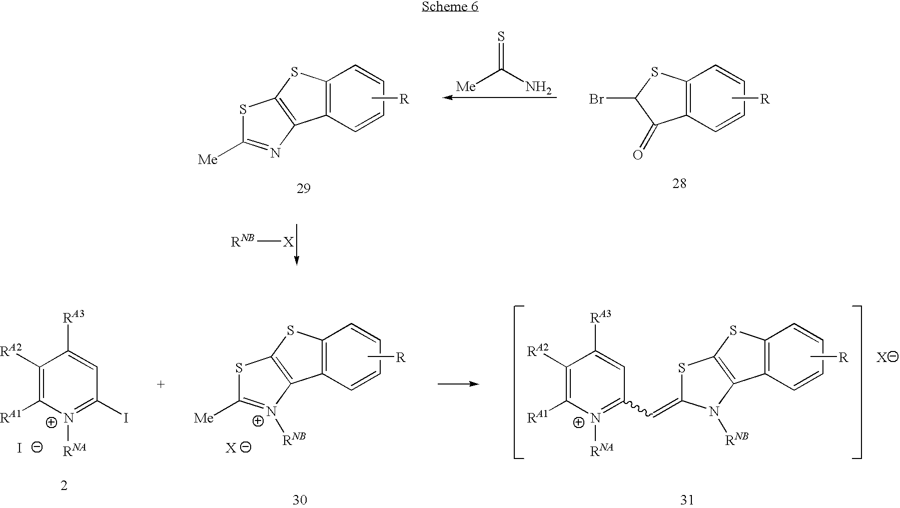

In another approach, conversion of benzofuran-2-carboxylic acid (32) to the corresponding acid azide (33) followed by rearrangement and reaction with a methyl Grignard reagent leads to formation of the corresponding amide (34). Subsequent reaction with Lawesson's reagent to give the thioamide (35) followed by oxidation to give the tricycle (36) and reaction with methyl iodide gives the corresponding reversed benzofuran ring (37) which can be used in subsequent synthesis. See, for example, Abramenko et al., 1977, for the synthesis of the benzofuranthiazole.

An example of such an approach is illustrated in the following scheme.

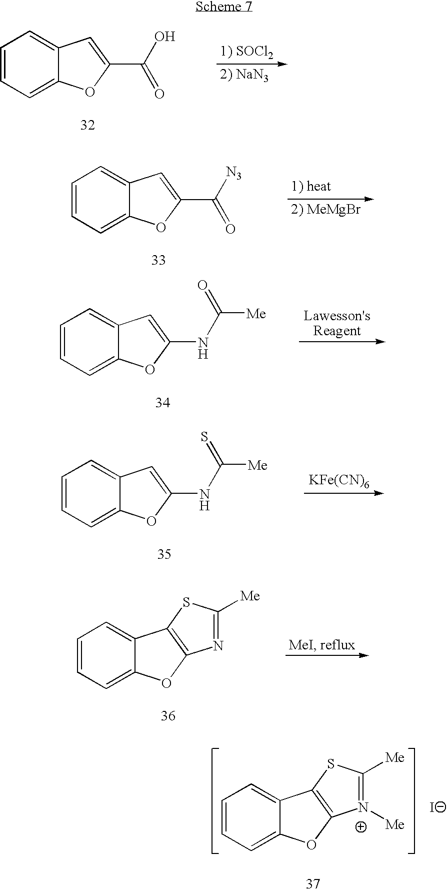

An alternative approach for the synthesis of quinolinium containing compounds involves reaction of a suitable N-ethyl-quinone with phosphorus oxychloride to give the corresponding N-ethyl chloro compound which can be used in subsequent synthesis.

An example of such an approach is illustrated in the following scheme.

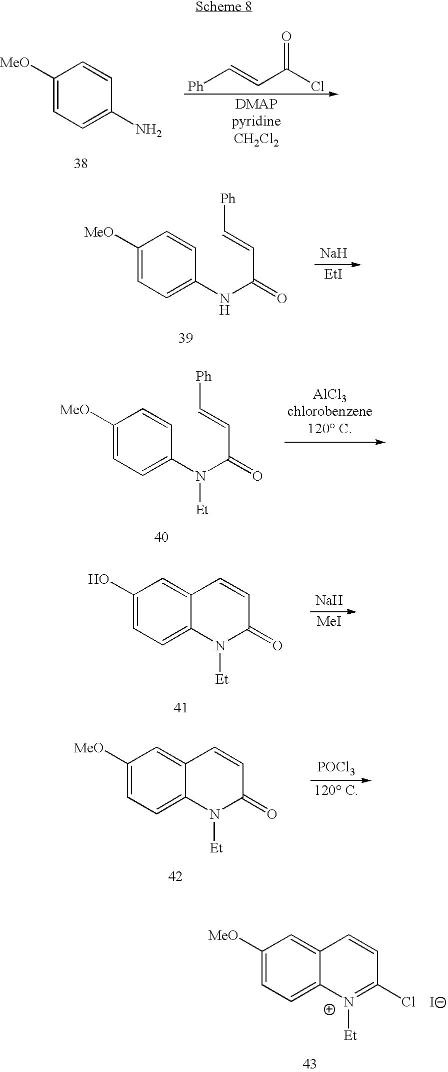

In one approach, the target compounds may be prepared by a method that involves the base promoted reaction between a halopyridinium salt with a methylthiazolium salt. For example, a fluoropyridinium tetrafluoroborate salt may itself be prepared by reaction of the corresponding 2-fluoropyridine with a suitable oxonium salt, in analogy to the methods described by Li et al., 2000.

An example of such an approach is illustrated in the following scheme.

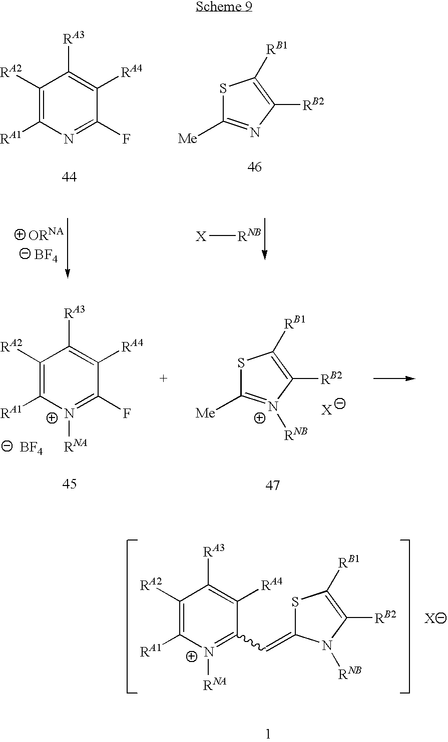

### Uses

The 2-[3H-thiazol-2-ylidinemethyl]pyridine compounds and analogs thereof, described herein, are useful, for example, in the treatment of proliferative conditions, such as cancer.

### Use in Methods of Inhibiting Cell Proliferation, Etc.

The compounds (i.e., 2-[3H-thiazol-2-ylidinemethyl]pyridine compounds and analogs thereof) described herein, e.g., (a) regulate (e.g., inhibit) cell proliferation; (b) inhibit cell cycle progression; (c) promote apoptosis; or (d) a combination of one or more of these.

One aspect of the present invention pertains to a method of regulating (e.g., inhibiting) cell proliferation (e.g., proliferation of a cell), inhibiting cell cycle progression, promoting apoptosis, or a combination of one or more these, in vitro or in vivo, comprising contacting cells (or the cell) with an effective amount of a compound, as described herein.

In one embodiment, the method is a method of regulating (e.g., inhibiting) cell proliferation (e.g., proliferation of a cell), in vitro or in vivo, comprising contacting cells (or the cell) with an effective amount of a compound, as described herein.

In one embodiment, the method is performed in vitro.

In one embodiment, the method is performed in vivo.

In one embodiment, the compound is provided in the form of a pharmaceutically acceptable composition.

Any type of cell may be treated, including but not limited to, lung, gastrointestinal (including, e.g., bowel, colon), breast (mammary), ovarian, prostate, liver (hepatic), kidney (renal), bladder, pancreas, brain, and skin.

One of ordinary skill in the art is readily able to determine whether or not a candidate compound regulates (e.g., inhibits) cell proliferation, etc. For example, assays that may conveniently be used to assess the activity offered by a particular compound are described in the examples below.

For example, a sample of cells (e.g., from a tumour) may be grown in vitro and a compound brought into contact with said cells, and the effect of the compound on those cells observed. As an example of “effect,” the morphological status of the cells (e.g., alive or dead, etc.) may be determined. Where the compound is found to exert an influence on the cells, this may be used as a prognostic or diagnostic marker of the efficacy of the compound in methods of treating a patient carrying cells of the same cellular type.

### Use in Methods of Therapy

Another aspect of the present invention pertains to a compound (i.e., a 2-[3H-thiazol-2-ylidinemethyl]pyridine compound or analog thereof, as described herein, for use in a method of treatment of the human or animal body by therapy.

### Use in the Manufacture of Medicaments

Another aspect of the present invention pertains to use of a compound (i.e., a 2-[3H-thiazol-2-ylidinemethyl]pyridine compound or analog thereof, as described herein, in the manufacture of a medicament for use in treatment.

### Methods of Treatment

Another aspect of the present invention pertains to a method of treatment comprising administering to a patient in need of treatment a therapeutically effective amount of a compound (i.e., a 2-[3H-thiazol-2-ylidinemethyl]pyridine compound or analog thereof), as described herein, preferably in the form of a pharmaceutical composition.

### Conditions Treated—Proliferative Conditions and Cancer (1)

The compounds of the present invention are useful in the treatment of proliferative conditions (as “anti-proliferative agents”), cancer (as “anti-cancer agents”), etc.

The term “antiproliferative agent” as used herein, pertains to a compound that treats a proliferative condition (i.e., a compound which is useful in the treatment of a proliferative condition). The terms “proliferative condition,” “proliferative disorder,” and “proliferative disease,” are used interchangeably herein and pertain to an unwanted or uncontrolled cellular proliferation of excessive or abnormal cells that is undesired, such as, neoplastic or hyperplastic growth.

The term “anticancer agent” as used herein, pertains to a compound that treats a cancer (i.e., a compound which is useful in the treatment of a cancer). The anti-cancer effect may arise through one or more mechanisms, including but not limited to, the regulation of cell proliferation, the inhibition of cell cycle progression, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death).

One of ordinary skill in the art is readily able to determine whether or not a candidate compound treats a proliferative condition, or treats cancer, for any particular cell type. For example, assays that may conveniently be used to assess the activity offered by a particular compound are described in the examples below.

Note that active compounds includes both compounds with intrinsic activity (drugs) as well as prodrugs of such compounds, which prodrugs may themselves exhibit little or no intrinsic activity.

In one embodiment (e.g., of use in methods of therapy, of use in the manufacture of medicaments, of methods of treatment), the treatment is treatment of a proliferative condition.

In one embodiment, the treatment is treatment of a proliferative condition characterised by benign, pre-malignant, or malignant cellular proliferation, including but not limited to, neoplasms, hyperplasias, and tumours (e.g., histocytoma, glioma, astrocyoma, osteoma), cancers (see below), psoriasis, bone diseases, fibroproliferative disorders (e.g., of connective tissues), pulmonary fibrosis, atherosclerosis, smooth muscle cell proliferation in the blood vessels, such as stenosis or restenosis following angioplasty.

In one embodiment, the treatment is treatment of cancer.

In one embodiment, the treatment is treatment of: lung cancer, small cell lung cancer, non-small cell lung cancer, gastrointestinal cancer, stomach cancer, bowel cancer, colon cancer, rectal cancer, colorectal cancer, thyroid cancer, breast cancer, ovarian cancer, endometrial cancer, prostate cancer, testicular cancer, liver cancer, kidney cancer, renal cell carcinoma, bladder cancer, pancreatic cancer, brain cancer, glioma, sarcoma, osteosarcoma, bone cancer, skin cancer, squamous cancer, Kaposi's sarcoma, melanoma, malignant melanoma, lymphoma, leukaemia, and tumours of unknown origin.

In one embodiment, the treatment is treatment of ovarian cancer.

In one embodiment, the treatment is treatment of colon cancer.

In one embodiment, the treatment is treatment of breast cancer.

In one embodiment, the treatment is treatment of prostate cancer.

In one embodiment, the treatment is treatment of melanoma.

In one embodiment, the treatment is treatment of non-small cell lung cancer.

All histological subtypes of the cancer above are included. For example, a pathologist may determine the histological subtype of a cancer based upon the cell morphology, for example, mucinous, adenocarcinoma, serous, papillary, etc.

In one embodiment, the treatment is treatment of:
a carcinoma, for example a carcinoma of the bladder, breast, colon (e.g., colorectal carcinomas such as colon adenocarcinoma and colon adenoma), kidney, epidermal, liver, lung (e.g., adenocarcinoma, small cell lung cancer and non-small cell lung cancer), oesophagus, gall bladder, ovary (e.g., epithelial ovarian cancer), pancreas (e.g., exocrine pancreatic carcinoma), stomach, cervix, thyroid, prostate, skin (e.g., squamous cell carcinoma);
a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma;
a hematopoietic tumor of myeloid lineage, for example acute and chronic myelogenous leukemias, myelodysplastic syndrome, or promyelocytic leukemia;
a tumour of mesenchymal origin, for example fibrosarcoma or rhabdomyosarcoma;
a tumor of the central or peripheral nervous system, for example astrocytoma, neuroblastoma, glioma or schwannoma;
melanoma; seminoma; teratocarcinoma; osteosarcoma; xenoderoma pigmentoum; keratoctanthoma; thyroid follicular cancer; or Kaposi's sarcoma.

In one embodiment, the treatment is treatment of solid tumour cancer.

In one embodiment, the treatment is treatment of: epithelial ovarian cancer, non-small cell lung cancer, small cell lung cancer, colorectal cancer, testicular cancer (e.g., relapsed testicular cancer), skin (e.g., head and neck) cancer.

The compounds (i.e., 2-[3H-thiazol-2-ylidinemethyl]pyridine compounds and analogs thereof) described herein may be used in the treatment of the cancers described herein, independent of the mechanisms discussed herein.

### Conditions Treated—Proliferative Conditions and Cancer (2)

In one embodiment (e.g., of use in methods of therapy, of use in the manufacture of medicaments, of methods of treatment), the treatment is treatment of a proliferative condition (e.g., cancer, tumour, etc.) as described above, that is additionally characterised by one or more or all of the following:

(a) cells (e.g., cancer cells, tumour cells) and/or tumours that have defective DNA mismatch repair (MMR) (e.g., loss of MMR);
(b) cells (e.g., cancer cells, tumour cells) and/or tumours that have acquired defective DNA MMR following chemotherapy;
(c) cells (e.g., cancer cells, tumour cells) and/or tumours that have microsatellite instability (MSI), and/or increased frameshift mutation frequency, and/or another measure of replication errors;
(d) the patient also having HNPCC syndrome (inherited defects in DNA MMR);
(e) cells (e.g., cancer cells, tumour cells) and/or tumours that have epigenetic and/or genetic changes/mutations/abnormalities at DNA MMR loci, such as hMSH2, hMSH6, hPMS2, hMSH3, hMLH1, hMLH3, and especially hMLH1;
(f) cells (e.g., cancer cells, tumour cells) and/or tumours that acquire epigenetic and/or genetic changes/mutations/abnormalities at DNA MMR loci, such as hMSH2, hMSH6, hPMS2, hMSH3, hMLH1, hMLH3, and especially hMLH1, following chemotherapy;
(g) cells (e.g., cancer cells, tumour cells) and/or tumours with reduced expression of DNA MMR genes and proteins;
(h) cells (e.g., cancer cells, tumour cells) and/or tumours with defects in MMR signalling pathways, such as defects in p73, p53, JNK, ATM, CHK1, and CHK2 dependent cell cycle and apoptosis control, especially p53;
(i) cells (e.g., cancer cells, tumour cells) and/or tumours that have acquired resistance to chemotherapy;
(j) cells (e.g., cancer cells, tumour cells) and/or tumours that have relapsed following prior treatment, especially following treatment with cytotoxic chemotherapeutics, such as platinum based chemotherapeutics, such as platinum coordination complexes, such as cisplatin or carboplatin; monofunctional alkylating agents such as temodol/temozolomide; purine analogues such as 6-thioguanine; and topoisomerase II inhibitors such as doxorubicin;
(k) cells (e.g., cancer cells, tumour cells) and/or tumours that have acquired resistance to treatment, especially following treatment with cytotoxic chemotherapeutics, such as platinum based chemotherapeutics, such as platinum coordination complexes, such as cisplatin or carboplatin (e.g., cells with acquired cisplatin or carboplatin resistance); monofunctional alkylating agents such as temodol/temozolomide; purine analogues such as 6-thioguanine; and topoisomerase II inhibitors such as doxorubicin;
(l) cells (e.g., cancer cells, tumour cells) and/or tumours that fail to respond to treatment with cytotoxic chemotherapeutics, such as platinum based chemotherapeutics, such as platinum coordination complexes, such as cisplatin or carboplatin; monofunctional alkylating agents such as temodol/temozolomide; purine analogues such as 6-thioguanine; and topoisomerase II inhibitors such as doxorubicin, after previously responding;
(m) cells (e.g., cancer cells, tumour cells) and/or tumours that are therapy-related, such as therapy-related leukaemias arising following chemotherapy.

Each sub-combination of (a) through (m) is explicitly disclosed herein as if it was specifically and individually recited.

In one embodiment (e.g., of use in methods of therapy, of use in the manufacture of medicaments, of methods of treatment), the treatment is treatment of a proliferative condition (e.g., cancer, tumour, etc.) as described above, and additionally is characterised by: loss of DNA mismatch repair (MMR) (e.g., as characterised by MSI, and/or mutation of DNA MMR genes, and/or epigenetic silencing of DNA MMR genes, and/or reduced expression of DNA MMR genes/proteins, etc.).

For example, in one embodiment, the treatment is treatment of a proliferative condition (e.g., cancer, tumour, etc.) as described above, and additionally is characterised by proliferative (e.g., cancer, tumour) cells characterised by loss of DNA mismatch repair (MMR).

For example, in one embodiment, the treatment is treatment of a proliferative condition (e.g., cancer, tumour, etc.) as described above, and additionally is characterised by proliferative (e.g., cancer, tumour) cells characterised by acquired cisplatin or carboplatin resistance.

In one embodiment (e.g., of use in methods of therapy, of use in the manufacture of medicaments, of methods of treatment), the treatment is treatment of a proliferative condition (e.g., cancer, tumour, etc.) as described above, and additionally is characterised by: acquisition of resistance to chemotherapy.

### Screening

Prior to treatment, a patient may be screened in order to determine whether a disease or condition from which the patient is or may be suffering is one which would be susceptible to treatment with a compound (i.e., a 2-[3H-thiazol-2-ylidinemethyl]pyridine compound or analog thereof) described herein.

For example, a patient may be screened:

(a) for defective DNA mismatch repair (MMR) activity in tumours or tissue;
(b) for microsatellite instability (MSI) in tumour, tissue, or body fluid DNA;
(c) for increased rate of frameshift mutations in tumour, tissue, or body fluid DNA;
(d) for increased replication errors in tumour, tissue, or body fluid DNA;
(e) for reduced expression of DNA MMR genes or proteins in tumours or secreted into body fluids;
(f) for genetic mutations at DNA MMR loci;
(g) for aberrant epigenetic regulation at DNA MMR loci, for example, increased 5-methyl-cytosine at CpG dinucleotides in DNA, or histone modifications;
(h) for in vitro sensitivity and acquired resistance of cells (including tumour cells and circulating cells) to cytotoxic chemotherapeutics;
(i) for altered levels of apoptosis or cell cycle progression, especially as measured following treatment in vivo or ex vivo of tumour cells with cytotoxic chemotherapeutics;
(j) according to the length of progression-free survival (PFS) (for example, selecting epithelial ovarian cancer patients with a PFS of less than 12 months);
(k) clinical drug resistance (e.g., patients who progress on treatment with chemotherapy).

Each sub-combination of (a) through (k) is explicitly disclosed herein as if it was specifically and individually recited.

For example, a biological sample taken from a patient may be analysed to determine whether a condition or disease, such as cancer, that the patient is or may be suffering from, is one which is characterised by defects in DNA MMR activity, or one which has acquired resistance to cytotoxic chemotherapeutics (e.g., platinum based chemotherapeutics).

Thus, the patient may be subjected to a diagnostic test to detect a marker characteristic of defective DNA MMR activity, especially as it relates to genomic instability (replication errors) or signalling pathways that modulate cell death and cell cycle progression.

Typical methods for screening for defective DNA mismatch repair (MMR) include, but are not limited to, screening for: (a) differences in the length of repeat sequences in tumour DNA as compared to normal DNA, (b) increased DNA methylation at CpG dinucleotides at or near DNA MMR genes, (c) differences in levels of mRNA and protein levels in tumours of DNA MMR genes, (d) mutations in DNA MMR genes, for example, as determined by DNA sequencing. These methods may also be applied, for example, to body fluids or DNA extracted from body fluids.

### Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, alleviation of symptoms of the condition, amelioration of the condition, and cure of the condition. Treatment as a prophylactic measure (i.e., prophylaxis) is also included. For example, use with patients who have not yet developed the condition, but who are at risk of developing the condition, is encompassed by the term “treatment.”

For example, treatment includes the prophylaxis of cancer, reducing the incidence of cancer, alleviating the symptoms of cancer, etc.

The term “therapeutically-effective amount,” as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen.

### Combination Therapies

The term “treatment” includes combination treatments and therapies, in which two or more treatments or therapies are combined, for example, sequentially or simultaneously. For example, the compounds described herein may also be used in combination therapies, e.g., in conjunction with other agents, for example, cytotoxic agents, anticancer agents, etc. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; photodynamic therapy; gene therapy; and controlled diets.

For example, it may be beneficial to combine treatment with a compound as described herein with one or more other (e.g., 1, 2, 3, 4) agents or therapies that regulates cell growth or survival or differentiation via a different mechanism, thus treating several characteristic features of cancer development. Examples of such combinations are set out below.

In one embodiment, a compound (i.e., a 2-[3H-thiazol-2-ylidinemethyl]pyridine compound or analog thereof) described herein is combined with one or more (e.g., 1, 2, 3, 4) additional therapeutic agents, as described below.

One aspect of the present invention pertains to a compound as described herein, in combination with one or more (e.g., 1, 2, 3, 4) additional therapeutic agents, as described below.

Examples of additional therapeutic agents that may be administered together (whether concurrently or at different time intervals) with the compounds described herein include:
(a) agents known to be less effective in MMR deficient cells, such as: cisplatin, carboplatin, 6-thioguanine, temozolomide, epirubicin, doxorubicin, 5-FU;
(b) other cytotoxic chemotherapeutics, such as oxaliplatin;
(c) epigenetic therapeutics, such as 5-aza-cytidine and derivatives thereof.

The particular combination would be at the discretion of the physician who would select dosages using his common general knowledge and dosing regimens known to a skilled practitioner.

The agents (i.e., the compound described here, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (i.e., the compound described here, plus one or more other agents) may be formulated together in a single dosage form, or alternatively, the individual agents may be formulated separately and presented together in the form of a kit, optionally with instructions for their use, as described below.

The agents (i.e., the compound described here, plus one or more other agents) may be administered alternately, for example, according to the onset of drug resistance. For example, one or more other agents may be administered until the onset of resistance to that agent; then treatment may be switched to treatment with a compound as described herein. Optionally, this pattern may be repeated (e.g., by switching to another treatment, e.g., with one or more other agents, e.g., the original other agent(s), again until the onset of resistance to that agent, and then again treatment may be switched to treatment with a compound as described herein.

### Other Uses

The compounds described herein may also be used as cell culture additives to inhibit cell proliferation, etc.

The compounds described herein may also be used as part of an in vitro assay, for example, in order to determine whether a candidate host is likely to benefit from treatment with the compound in question.

The compounds described herein may also be used as a standard, for example, in an assay, in order to identify other active compounds, other anti-proliferative agents, other anti-cancer agents, etc.

### Kits

One aspect of the invention pertains to a kit comprising (a) an active compound as described herein, or a composition comprising an active compound as described herein, e.g., preferably provided in a suitable container and/or with suitable packaging; and

(b) instructions for use, e.g., written instructions on how to administer the active compound or composition.

The written instructions may also include a list of indications for which the active ingredient is a suitable treatment.

### Routes of Administration

The active compound or pharmaceutical composition comprising the active compound may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., using an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### The Subject/Patient

The subject/patient may be an animal, a mammal, a placental mammal, a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus.

In one preferred embodiment, the subject/patient is a human.

### Formulations

While it is possible for the active compound to be administered alone, it is preferable to present it as a pharmaceutical formulation (e.g., composition, preparation, medicament) comprising at least one active compound, as defined above, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, as defined above, and methods of making a pharmaceutical composition comprising admixing at least one active compound, as defined above, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the active compound.

The term “pharmaceutically acceptable” as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, lozenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more active compounds and optionally one or more other pharmaceutically acceptable ingredients, including, for example, penetration, permeation, and absorption enhancers. Formulations may also suitably be provided in the form of a depot or reservoir.

The active compound may be dissolved in, suspended in, or admixed with one or more other pharmaceutically acceptable ingredients. The active compound may be presented in a liposome or other microparticulate which is designed to target the active compound, for example, to blood components or one or more organs.

Formulations suitable for oral administration (e.g., by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses.

Formulations suitable for buccal administration include mouthwashes, lozenges, pastilles, as well as patches, adhesive plasters, depots, and reservoirs. Lozenges typically comprise the active compound in a flavored basis, usually sucrose and acacia or tragacanth. Pastilles typically comprise the active compound in an inert matrix, such as gelatin and glycerin, or sucrose and acacia. Mouthwashes typically comprise the active compound in a suitable liquid carrier.

Formulations suitable for sublingual administration include tablets, lozenges, pastilles, capsules, and pills.

Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, lozenges, pastilles, as well as patches, adhesive plasters, depots, and reservoirs.

Formulations suitable for non-oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the active compound and a paraffinic or a water-miscible ointment base.

Creams are typically prepared from the active compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

Emulsions are typically prepared from the active compound and an oily phase, which may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, and (for aerosol administration by nebuliser) aqueous or oily solutions of the active compound.

Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for ocular administration include eye drops wherein the active compound is dissolved or suspended in a suitable carrier, especially an aqueous solvent for the active compound.

Formulations suitable for rectal administration may be presented as a suppository with a suitable base comprising, for example, natural or hardened oils, waxes, fats, semi-liquid or liquid polyols, for example, cocoa butter or a salicylate; or as a solution or suspension for treatment by enema.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g., by injection), include aqueous or non-aqueous, isotonic, pyrogen-free, sterile liquids (e.g., solutions, suspensions), in which the active compound is dissolved, suspended, or otherwise provided (e.g., in a liposome or other microparticulate). Such liquids may additionally contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the active compound in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the active compounds, and compositions comprising the active compounds, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the active compound is in the range of about 10 μg/m² to 1 g/m² per treatment (e.g., per day), more typically 1 mg/m² to 500 mg/m² per treatment (e.g., per day). Where the active compound is a salt, an ester, an amide, a prodrug, or the like, the amount administered is calculated on the basis of the parent compound and so the actual weight to be used is increased proportionately.

### EXAMPLES

The following are examples are provided solely to illustrate the present invention and are not intended to limit the scope of the invention, as described herein.

### Synthesis 1

### 2-Iodoquinoline ethiodide (009)

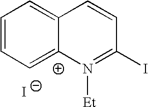

2-Chloroquinoline (1.45 g, 8.9 mmol) was dissolved in iodoethane (3.0 mL, 37.6 mmol) and the resultant solution was stirred at reflux for 10 days. After cooling to room temperature, the mixture was filtered and the solid collected was washed with acetone (2×5 mL), to give the crude product as a yellow/orange solid (1.95 g), which was recrystallised from MeOH/Et₂O, to give the title compound as orange needles (1.02 g, 36%). ¹H-NMR (500 MHz, D₃COD): 1.71-1.73 (m, 3H, CH₃CH₂N); 5.38 (br, 2H, CH₃CH₂N); 8.03-8.05 (m, 1H, Ar—H); 8.26 (t, 1H, J=7.1, Ar—H); 8.39 (d, 1H, J=7.7, Ar—H); 8.62-8.69 (m, 3H, Ar—H). ESI-MS (pos., MeOH): 284 (100, [M-I]⁺).

### Synthesis 2

### 6-Methoxy-2-methylbenzothiazole methiodide (001)

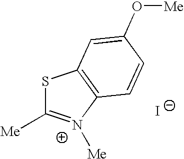

Method AA: To a stirred solution of 6-methoxy-2-methylbenzothiazole (1.00 g, 5.58 mmol) in acetone (3 mL) was added iodomethane (1.04 mL, 16.74 mmol) and the solution was stirred at reflux for 20 hours. After cooling to room temperature, the mixture was filtered, and the solid collected was washed with acetone (3×3 mL), to give the crude product as a white, semi-crystalline solid (1.41 g), which was recrystallised from MeOH to give the title compound as white crystals (1.05 g, 59%). ¹H-NMR (500 MHz, d-DMSO): 3.10 (s, 3H, CH₃C═N); 3.90 (s, 3H, OCH₃); 4.15 (s, 3H, NCH₃); 7.48 (br d, 1H, J=9.2, Ar—H); 7.99 (br s, 1H, Ar—H); 8.18 (d, 1H, J=9.2, Ar—H). ESI-MS (pos., MeOH): 194 (100, [M-I]⁺).

### Synthesis 3

### Cyanine Dye (027)

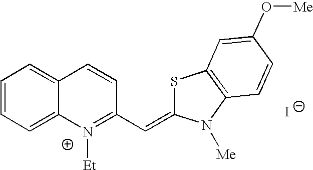

2-Iodoquinoline ethiodide (85 mg, 0.21 mmol) and 6-methoxy-2-methylbenzothiazole methiodide (70 mg, 0.22 mmol) were dissolved in a mixture of MeCN (3 mL) and MeOH (1 mL), and this orange solution was stirred at 40° C. Et₃N (92 μL, 0.66 mmol) was added dropwise. A red precipitate formed immediately. The mixture was stirred for 30 minutes, then filtered, and the solid collected was washed with MeCN (2 mL), to give a red, clay-like solid, which was dried to constant weight to give the title compound (50 mg, 53%). ¹H-NMR (500 MHz, d-DMSO): 1.42 (t, 3H, J=7.1, CH₃CH₂N); 3.78 (s, 3H, OCH₃); 3.88 (s, 3H, CH₃N); 4.59 (br, 2H, CH₃CH₂N); 5.95 (s, 1H, C═CH); 7.15 (dd, 1H, J=9.0, 2.5, Ar—H); 7.52 (t, 1H, J=7.5, Ar—H); 7.62 (d, 1H, J=2.5, Ar—H); 7.69 (d, 1H, J=9.1, Ar—H); 7.82 (d, 1H, J=7.9, Ar—H); 7.92 (d, 2H, J=9.2, Ar—H); 7.99 (d, 1H, J=8.8, Ar—H); 8.30 (d, 1H, J=9.4, Ar—H). ESI-MS (pos., MeOH): 349 (100, [M-I]⁺). UV-vis λₘₐₓ=484 nm (MeOH, ε=4.37×10⁴).

### Synthesis 4

### Quinaldine Ethiodide (045)

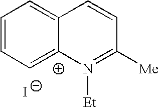

Method BB: Quinaldine (2.0 g, 14.0 mmol) was dissolved in iodoethane (6 mL, 75.2 mmol) and the resultant solution was stirred at reflux for 18 hours. After cooling to room temperature, the mixture was filtered and the solid collected was washed with acetone (3×10 mL) to give the title compound as a yellowish solid (2.11 g, 50%). ¹H-NMR (500 MHz, d-DMSO): 1.53 (t, 3H, J=7.0, CH₃CH₂N); 3.11 (s, 3H, N═CCH₃); 5.00 (q, 2H, J=7.0, CH₃CH₂N); 8.00 (t, 1H, J=7.5, Ar—H); 8.13 (d, 1H, J=8.4, Ar—H); 8.24 (dd, 1H, J=7.9, 7.5, Ar—H); 8.42 (d, 1H, J=7.9, Ar—H); 8.62 (d, 1H, J=8.9, Ar—H); 9.11 (d, 1H, J=8.4, Ar—H). ESI-MS (pos., MeOH): 172 (100, [M-I]⁺).

### Synthesis 5

### 2-(Methylthio)benzothiazole methyl-1-toluenesulfonate (051)

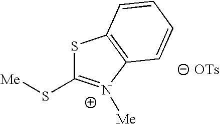

Method CC: To a stirred solution of 2-(methylthio)benzothiazole (5.0 g, 27.6 mmol) in DMF (10 mL) was added methyl p-toluenesulfonate (7.7 g, 41.4 mmol) and the colourless solution was stirred at 120° C. for 2.5 hours. After cooling to room temperature, acetone (50 mL) was added, and the mixture was left overnight. The mixture was filtered and the solid collected was washed with acetone (2×20 mL). The solid was suspended in acetone (60 mL) and stirred at reflux for 1 hour. After cooling to room temperature, the mixture was filtered and the solid collected was washed with acetone (3×30 mL), to yield the title compound as a white solid (9.0 g, 89%). ¹H-NMR (500 MHz, D₃COD): 2.37 (s, 3H, CH₃Ph); 3.14 (s, 3H, SCH₃); 4.16 (s, 3H, NCH₃); 7.22 (d, 2H, J=7.8, Ar—H); 7.69 (d, 2H, J=8.1, Ar—H); 7.75 (t, 1H, J=7.7, Ar—H); 7.86 (t, 1H, J=7.9, Ar—H); 8.10 (d, 1H, J=8.5, Ar—H); 8.23 (d, 1H, J=8.1, Ar—H). ESI-MS (pos., MeOH): 196 (100, [M-OTs]⁺).

### Synthesis 6

### Cyanine Dye (053)

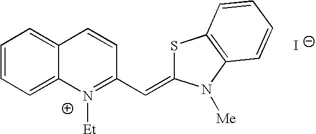

Method DD: 2-(Methylthio)benzothiazole methyl-p-toluenesulfonate (300 mg, 0.81 mmol) and quinaldine ethiodide (243 mg, 0.81 mmol) were dissolved in a mixture of MeCN (6 mL) and MeOH (1 mL) at 70° C. To this stirred solution at 70° C. was added dropwise Et₃N (340 μL, 2.43 mmol). A dark precipitate was formed immediately. The mixture was stirred at 70° C. for 1.5 hours, then EtOAc (7 mL) was added and the mixture was cooled to room temperature. The mixture was filtered and the solid collected was washed with EtOAc (2×5 mL), to give a dark red solid (215 mg) which was recrystallised from MeOH/EtOAc to yield the title compound as dark red needles, which were dried to constant weight (99 mg, 27%). ¹H-NMR (500 MHz, d-DMSO): 1.52 (t, 3H, J=6.8, CH₃CH₂N); 3.96 (s, 3H, CH₃N); 4.72 (br, 2H, CH₃CH₂N); 6.10 (s, 1H, C═CH); 7.44 (t, 1H, J=7.60, Ar—H); 7.60-7.64 (m, 2H, Ar—H); 7.81 (d, 1H, J=8.2, Ar—H); 7.93 (t, 1H, J=7.9, Ar—H); 8.02-8.06 (m, 3H, Ar—H); 8.12 (d, 1H, J=8.7, Ar—H); 8.45 (d, 1H, J=9.3, Ar—H). ¹³C-NMR (125 MHz, d-DMSO): 12.03 (CH₃CH₂N); 33.87 (CH₃N); 43.99 (CH₃CH₂N); 85.29 (C═CH); 113.11, 116.88, 118.61, 122.84, 123.38, 124.21, 124.83, 125.67, 133.63, 138.63, 139.98, 140.71, 152.06, 161.97 (Arom. C). UV-vis λₘₐₓ=484 nm (MeOH, ε=3.54×10⁴). ESI-MS (pos., MeOH): 320 (100, [M-I]⁺).

### Synthesis 7

### 2-Bromobenzofuran-3-(2H)-one (091)

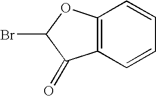

To a stirred mixture of benzofuran-3-(2H)-one (1.50 g, 11.2 mmol) in THF (20 mL) at −25° C. was added dropwise over 15 minutes a solution of trimethylphenylammonium tribromide (4.43 g, 11.9 mmol) in THF (15 mL). The mixture was stirred for 1 hour, keeping the temperature between −10 and -25° C. The mixture was poured into saturated aqueous NaHCO₃ (90 mL) and H₂O (90 mL), and this mixture was extracted with EtOAc (3×60 mL). The combined organic extracts were washed with brine (20 mL), dried (MgSO₄), and evaporated to give a crude solid (2.48 g), which was purified by flash chromatography on silica gel (eluent: petroleum ether with 1.5% Et₂O) to give the title compound as a yellowish solid (1.50 g, 63%). ¹H-NMR (500 MHz, CDCl₃): 6.51 (s, 1H, CHBr); 7.18 (d, 1H, J=8.3, Ar—H); 7.23 (t, 1H, J=7.6, Ar—H); 7.70 (t, 1H, J=8.3, Ar—H), 7.78 (d, 1H, J=7.6, Ar—H).

### Synthesis 8

### 2-Methylbenzofuro-[3,2-d]thiazole (041)

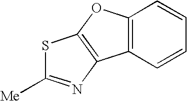

To a stirred solution of thioacetamide (79 mg, 0.92 mmol) in EtOH (2 mL) at 5° C. was added 2-bromobenzofuran-3-(2H)-one (195 mg, 0.92 mmol). A white precipitate formed after 5 minutes, and the mixture was immediately filtered. The white solid collected was washed with toluene (5 mL). The solid was added in small portions to concentrated H₂SO₄ (600 μL) with stirring at room temperature. This red mixture was stirred for 10 minutes, then poured into ice (6 g) to give a yellow cloudy mixture. After stirring at 10 minutes, the mixture was neutralised to pH 6 with 10% aqueous NaOH (8 mL). The mixture was then filtered and the solid collected was washed with H₂O (2×1 mL), to give the title compound as a yellowish solid, which was dried to constant weight (82 mg, 47%). ¹H-NMR (500 MHz, CDCl₃): 2.85 (s, 3H, CH₃); 7.34-7.37 (m, 2H, Ar—H); 7.57 (d, 1H, J=7.1, Ar—H); 7.90 (d, 1H, J=7.1, Ar—H).

### Synthesis 9

### 2-Methylbenzofuro-[3,2-d]thiazole methiodide (043)

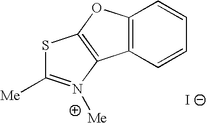

To a solution of 2-methylbenzofuro-[3,2-d]thiazole (93 mg, 0.49 mmol) in acetone (1 mL) was added iodomethane (607 μL, 4.9 mmol) and the resultant solution was stirred at reflux for 48 hours. A precipitate formed during this time. After cooling to room temperature, the mixture was filtered and the solid collected was washed with acetone (2×2 mL), to give the title compound as a grey solid (91 mg, 56%). ¹H-NMR (250 MHz, D₃COD): 3.14 (s, 3H, CH₃); 4.46 (s, 3H, NCH₃); 7.55-7.70 (m, 2H, Ar—H); 7.76 (d, 1H, J=7.5, Ar—H); 8.17 (d, 1H, J=7.5, Ar—H). ESI-MS (pos., MeOH): 204 (100, [M-I]⁺).

### Synthesis 10

### Cyanine Dye (086)

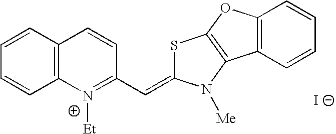

To a stirred solution of 2-iodoquinoline ethiodide (37 mg, 0.09 mmol) and 2-methylbenzofuro-[3,2-d]thiazole methiodide (30 mg, 0.09 mmol) in DMF (0.9 mL) at room temperature was added dropwise Et₃N (32 μL, 0.23 mmol). A purple precipitate formed immediately. The mixture was stirred for 1.5 hours, then acetone (15 mL) was added, and the mixture was filtered. The solid collected was washed with acetone (2 mL), to give the title compound as a purple, clay-like solid, which was dried to constant weight (11 mg, 25%). ¹H-NMR (500 MHz, d-DMSO): 1.63 (t, 3H, J=7.2, CH₃CH₂N); 4.21 (s, 3H, CH₃N); 4.64 (br, 2H, CH₃CH₂N); 5.94 (s, 1H, C═CH); 7.54 (m, 3H, Ar—H); 7.77 (d, 1H, J=8.5, Ar—H); 7.88-7.99 (m, 4H, Ar—H); 8.08 (d, 1H, J=7.5, Ar—H); 8.19 (d, 1H, J=9.3, Ar—H). ESI-MS (pos., MeOH): 359 (100, [M-I]⁺). UV-vis λₘₐₓ=499 nm (MeOH, ε=6.60×10⁴).

### Synthesis 11

### 2-(Methylthio)thiazole (073)

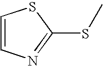

Method EE: 2-Mercaptothiazole (2.50 g, 21.3 mmol) was dissolved in DMF (25 mL) and to the resultant brown solution was added solid K₂CO₃ (3.54 g, 25.6 mmol). The mixture was stirred at room temperature for 30 minutes, then iodomethane (1.35 mL, 21.7 mmol) was added and the mixture stirred at room temperature for 30 minutes. The mixture was then diluted with EtOAc (25 mL) and water (20 mL). The aqueous layer was extracted with EtOAc (10 mL), and the combined organic extracts were washed with water (2×20 mL), brine (15 mL), dried (MgSO₄) and concentrated to give the title compound as a brown oil (1.94 g, 69%). ¹H-NMR (500 MHz, CDCl₃): 2.71 (s, 3H, CH₃); 7.20 (d, 1H, J=2.6, Ar—H); 7.66 (d, 1H, J=2.6, Ar—H).

### Synthesis 12

### 2-(Methylthio)thiazole methyl-p-toluenesulfonate (077)

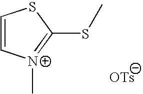

Using a method analogous to Method CC, with 2-(methylthio)thiazole (073) (1.93 g, 14.7 mmol) and methyl p-toluenesulfonate (3.33 mL, 22.1 mmol), the title compound was obtained as a grey semi-crystalline solid (4.70 g, quant.).

¹H-NMR (500 MHz, D₃COD): 2.38 (s, 3H, PhCH₃); 3.01 (s, 3H SCH₃); 3.99 (s, 3H, NCH₃); 7.24 (d, 2H, J=7.9, Ar—H); 7.71 (d, 2H, J=7.9, Ar—H); 7.98 (d, 1H, J=4.2, Ar—H); 8.16 (d, 1H, J=4.2, Ar—H).

### Synthesis 13

### 4-Methyl-2-(methylthio)thiazole (079)

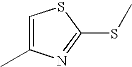

Using a method analogous to Method EE, with 4-methylthiazole-2-thiol (3.00 g, 22.9 mmol), solid K₂CO₃ (3.79 g, 27.5 mmol) and iodomethane (1.50 mL, 24.0 mmol), the title compound was obtained as a white semi-crystalline solid (2.35 g, 71%). ¹H-NMR (500 MHz, CDCl₃): 2.40 (s, 3H, CCH₃); 2.67 (s, 3H, SCH₃); 6.75 (s, 1H, Ar—H).

### Synthesis 14

### 4-Methyl-2-(methylthio)thiazole methyl-p-toluenesulfonate (080)

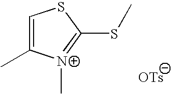

Using a method analogous to Method CC, with 4-Methyl-2-(methylthio)thiazole (079) (2.30 g, 15.9 mmol) and methyl p-toluenesulfonate (3.59 mL, 23.8 mmol), the title compound was obtained as a white crystalline solid (4.05 g, 77%). ¹H-NMR (500 MHz, d-DMSO): 2.28 (s, 3H, PhCH₃); 2.46 (s, 3H, CCH₃); 2.95 (s, 3H, SCH₃); 3.77 (s, 3H, NCH₃); 7.10 (d, 2H, J=8.0, Ar—H); 7.48 (d, 2H, J=8.0, Ar—H); 7.80 (s, 1H, Ar—H).

### Synthesis 15

### 5-Methoxy-2-(methylthio)benzothiazole (113)

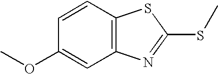

Using a method analogous to Method EE, with 2-mercapto-5-methoxy-benzothiazole (1.00 g, 5.09 mmol), solid K₂CO₃ (840 mg, 6.10 mmol), and iodomethane (332 μL, 5.32 mmol), the title compound was obtained as a white semi-crystalline solid (950 mg, 89%). ¹H-NMR (500 MHz, CDCl₃): 2.80 (s, 3H, SCH₃); 3.88 (s, 3H, OCH₃); 6.95 (dd, 1H, J=8.7, 2.5, Ar—H); 7.40 (d, 1H, J=2.5, Ar—H); 7.61 (d, 1H, J=8.7, Ar—H).

### Synthesis 16

### 5-Methoxy-2-(methylthio)benzothiazole methyl-p-toluenesulfonate (121)

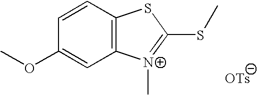

Using a method analogous to Method CC, with 5-methoxy-2-(methylthio) benzothiazole (113) (900 mg, 4.27 mmol) and methyl p-toluenesulfonate (770 μL, 5.12 mmol), the title compound was obtained as a white solid (1.21 g, 71%). ¹H-NMR (500 MHz, d-DMSO): 2.28 (s, 3H, PhCH₃); 3.08 (s, 3H, SCH₃); 3.94 (s, 3H, OCH₃); 4.08 (s, 3H, NCH₃); 7.10 (d, 2H, J=8.0, Ar—H); 7.34 (d, 1H, J=9.0, Ar—H); 7.46 (d, 2H, J=8.0, Ar—H); 7.71 (s, 1H, Ar—H); 8.24 (d, 1H, J=9.0, Ar—H).

### Synthesis 17

### 5-Chloro-2-(methylthio)benzothiazole (132)

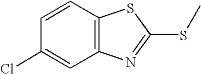

Using a method analogous to Method EE, with 5-chloro-2-mercapto benzothiazole (1.50 g, 7.44 mmol), solid K₂CO₃ (1.23 g, 8.93 mmol), and iodomethane (484 μL, 7.82 mmol), the title compound was obtained as a brownish semi-crystalline solid (1.60 g, quant.). ¹H-NMR (500 MHz, CDCl₃): 2.80 (s, 3H, SCH₃); 7.28 (d, 1H, J=8.5, Ar—H); 7.66 (d, 1H, J=8.5, Ar—H); 7.86 (s, 1H, Ar—H).

### Synthesis 18

### 5-Chloro-2-(methylthio)benzothiazole methyl-p-toluenesulfonate (136)

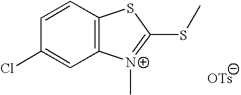

Using a method analogous to Method CC, with 5-Chloro-2-(methylthio) benzothiazole (132) (1.60 g, 7.44 mmol) and methyl p-toluenesulfonate (1.57 mL, 10.4 mmol), the title compound was obtained as a white solid (2.23 g, 77%). ¹H-NMR (500 MHz, d-DMSO): 2.27 (s, 3H, PhCH₃); 3.11 (s, 3H, SCH₃); 4.07 (s, 3H, NCH₃); 7.08 (d, 2H, J=7.9, Ar—H); 7.46 (d, 2H, J=7.9, Ar—H); 7.78 (d, 1H, J=8.8, Ar—H); 8.38 (d, 1H, J=8.8, Ar—H); 8.41 (s, 1H, Ar—H).

### Synthesis 19

### 2-Picoline ethiodide (083)

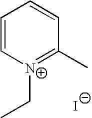

Using a method analogous to Method BB, with 2-picoline (2.83 g, 30.4 mmol) and iodoethane (10 mL, excess), the title compound was obtained as an orange crystalline solid (6.59 g, 93%). ¹H-NMR (500 MHz, D₃COD): 1.62 (t, 3H, J=7.4, CH₂CH₃); 2.93 (s, 3H, CCH₃); 4.68 (q, 2H, J=7.4, CH₂); 7.96 (dd, 1H, J=7.0, 6.3, Ar—H); 8.03 (d, 1H, J=7.9, Ar—H); 8.46 (dd, 1H, J=7.9, 7.0, Ar—H); 8.97 (d, 1H, J=6.3, Ar—H).

### Synthesis 20

### 2-Methylbenzothiazole methiodide (059)

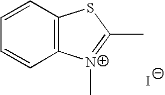

Using a method analogous to Method M, with 2-methylbenzothiazole (3.00 g, 20.0 mmol) and iodomethane (3.7 mL, 60 mmol), the title compound was obtained as a white solid (4.00 g, 69%). ¹H-NMR (500 MHz, d-DMSO): 3.19 (s, 3H, CCH₃); 4.21 (s, 3H, SCH₃); 7.80 (t, 1H, J=7.4, Ar—H); 7.89 (t, 1H, J=7.4, Ar—H); 8.29 (d, 1H, J=8.3, Ar—H); 8.46 (d, 1H, J=8.3, Ar—H).

### Synthesis 21

### Cyanine Dye (150A2)

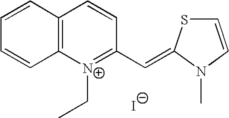

Using a method analogous to Method DD, with 2-(methylthio)thiazole methyl-p-toluenesulfonate (077) and quinaldine ethiodide (045), the title compound was obtained. ¹H-NMR (500 MHz, d-DMSO): 1.44 (t, 3H, J=7.1, CH₃CH₂N); 3.89 (s, 3H, CH₃N); 4.52 (br, 2H, CH₃CH₂N); 5.79 (s, 1H, C═CH); 7.41 (d, 1H, J=4.1, Ar—H); 7.48 (d, 1H, J=7.5, Ar—H); 7.72 (d, 1H, J=9.5, Ar—H); 7.80 (t, 1H, J=7.9, Ar—H); 7.84 (d, 1H, J=4.1, Ar—H); 7.88 (d, 1H, J=7.7, Ar—H); 7.91 (d, 1H, J=8.7, Ar—H); 8.17 (d, 1H, J=9.4, Ar—H). ¹³C-NMR (125 MHz, d-DMSO): 11.35 (CH₃CH₂N); 37.57 (CH₃N); 42.91 (CH₃CH₂N); 83.57 (C═CH); 109.54, 115.96, 117.78, 123.08, 124.41, 129.36, 132.98, 134.61, 138.11, 138.79, 149.52, 162.84 (Arom. C). UV-vis λₘₐₓ=465 nm (MeOH, ε=3.53×10⁴).

### Synthesis 22

### Cyanine Dye (150A3)

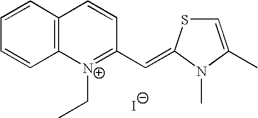

Using a method analogous to Method DD with 2-(methylthio)-4-methylthiazole methyl-p-toluenesulfonate (080) and quinaldine ethiodide (045), the title compound was obtained. ¹H-NMR (500 MHz, d-DMSO): 1.43 (t, 3H, J=7.9, CH₃CH₂N); 2.41 (s, 3H, CCH₃); 3.75 (s, 3H, CH₃N); 4.51 (br, 2H, CH₃CH₂N); 5.81 (s, 1H, C═CH); 7.05 (d, 1H, J=8.8, Ar—H); 7.37 (s, 1H, Ar—H); 7.60 (d, 1H, J=7.4, Ar—H); 7.89 (d, 1H, J=8.8, Ar—H); 7.92 (t, 1H, J=8.5, Ar—H); 8.01 (d, 2H, J=9.3, Ar—H); 8.09 (d, 1H, J=8.6, Ar—H); 8.41 (d, 1H, J=9.4, Ar—H). ¹³C-NMR (125 MHz, d-DMSO): 11.35 (CH₃CH₂N); 14.20 (CCH₃); 35.02 (CH₃N); 42.79 (CH₃CH₂N); 84.28 (C═CH); 105.18, 115.87, 118.16, 123.06, 124.29, 132.89, 137.83, 138.85, 141.50, 149.52, 163.75 (Arom. C). UV-vis λₘₐₓ=436 nm (MeOH, ε=3.57×10⁴).

### Synthesis 23

### Cyanine Dye (150A4)

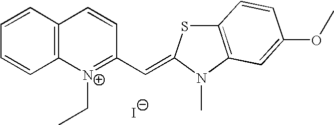

Using a method analogous to Method DD, with 2-(methylthio)-5-methoxybenzothiazole methyl-p-toluenesulfonate (121) and quinaldine ethiodide (045), the title compound was obtained. ¹H-NMR (500 MHz, d-DMSO): 1.51 (t, 3H, J=7.1, CH₃CH₂N); 3.91 (s, 3H, OCH₃); 3.96 (s, 3H, CH₃N); 4.70 (br, 2H, CH₃CH₂N); 6.07 (s, 1H, C); 7.05 (d, 1H, J=8.8, Ar—H); 7.37 (s, 1H, Ar—H); 7.60 (d, 1H, J=7.4, Ar—H); 7.89 (d, 1H, J=8.8, Ar—H); 7.92 (t, 1H, J=8.5, Ar—H); 8.01 (d, 2H, J=9.3, Ar—H); 8.09 (d, 1H, J=8.6, Ar—H); 8.41 (d, 1H, J=9.4, Ar—H). ¹³C-NMR (125 MHz, d-DMSO): 11.97 (CH₃CH₂N); 33.99 (CH₃N); 43.87 (CH₃CH₂N); 56.03 (OCH₃); 85.36 (C═CH); 98.43, 112.67, 114.54, 116.76, 118.57, 123.41, 124.04, 125.54, 129.61, 133.58, 138.63, 139.74, 142.06, 151.65, 160.06, 162.93 (Arom. C). UV-vis λₘₐₓ=491 nm (MeOH, ε=3.93×10⁴).

### Synthesis 24

### Cyanine Dye (150A5)

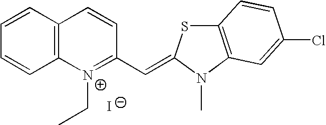

Using a method analogous to Method DD, with 2-(methylthio)-5-chlorobenzothiazole methyl-p-toluenesulfonate (136) and quinaldine ethiodide (045), the title compound was obtained. ¹H-NMR (500 MHz, d-DMSO): 1.52 (t, 3H, J=7.2, CH₃CH₂N); 3.92 (s, 3H, CH₃N); 4.75 (br, 2H, CH₃CH₂N); 6.09 (s, 1H, C═CH); 7.45 (d, 1H, J=8.5, Ar—H); 7.65 (d, 1H, J=8.2, Ar—H); 7.92-7.98 (m, 3H, Ar—H); 8.01 (d, 1H, J=8.5, Ar—H); 8.06 (d, 1H, J=8.3, Ar—H); 8.15 (d, 1H, J=8.8, Ar—H); 8.51 (d, 1H, J=9.3, Ar—H). ¹³C-NMR (125 MHz, d-DMSO): 12.17 (CH₃CH₂N); 34.01 (CH₃N); 44.29 (CH₃CH₂N); 85.77 (C═CH); 112.97, 117.07, 118.62, 122.19, 124.05, 124.44, 126.00, 129.75, 132.99, 133.84, 138.59, 140.51, 142.05, 152.35, 162.30 (Arom. C, 1 aromatic signal not found). UV-vis λₘₐₓ=483 nm (MeOH, ε=5.60×10⁴).

### Synthesis 25

### Cyanine Dye (150B2)

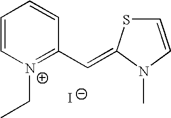

Using a method analogous to Method DD, with 2-(methylthio)thiazole methyl-p-toluenesulfonate (077) and 2-picoline ethiodide (083), the title compound was obtained. ¹H-NMR (500 MHz, d-DMSO): 1.41 (t, 3H, J=7.6, CH₃CH₂N); 3.71 (s, 3H, CH₃N); 4.86 (br, 2H, CH₃CH₂N); 5.55 (s, 1H, C═CH); 6.95 (t, 1H, J=8.0, Ar—H); 7.21 (d, 1H, J=4.0, Ar—H); 7.52 (d, 1H, J=8.0, Ar—H); 7.61 (d, 1H, J=4.0, Ar—H); 7.93 (t, 1H, J=8.0, Ar—H); 8.29 (d, 1H, J=8.0, Ar—H). ¹³C-NMR (125 MHz, d-DMSO): 12.02 (CH₃CH₂N); 35.61 (CH₃N); 49.56 (CH₃CH₂N); 77.94 (C═CH); 104.28, 112.96, 117.89, 132.39, 139.33, 141.59, 148.16, 158.63 (Arom. C). UV-vis λₘₐₓ=441 nm (MeOH, ε=2.12×10⁴).

### Synthesis 26

### Cyanine Dye (150B3)

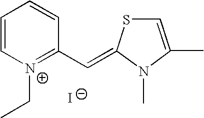

Using a method analogous to Method DD, with 2-(methylthio)-4-methylthiazole methyl-p-toluenesulfonate (080) and 2-picoline ethiodide (083), the title compound was obtained. ¹H-NMR (500 MHz, d-DMSO): 1.41 (t, 3H, J=7.5, CH₃CH₂N); 2.33 (s, 3H, CCH₃); 3.58 (s, 3H, CH₃N); 4.38 (q, 2H, J=7.5, CH₃CH₂N); 5.78 (s, 1H, C═CH); 6.84 (s, 1H, Ar—H); 6.95 (t, 1H, J=8.0, Ar—H); 7.56 (d, 1H, J=8.8, Ar—H); 7.92 (d, 1H, J=8.2, Ar—H); 8.28 (d, 1H, J=6.6, Ar—H). ¹³C-NMR (125 MHz, d-DMSO): 13.14 (CH₃CH₂N); 14.12 (CCH₃); 34.11 (CH₃N); 50.64 (CH₃CH₂N); 79.76 (C═CH); 100.93, 113.90, 119.31, 140.06, 140.35, 142.74, 149.25, 160.57 (Arom. C). UV-vis λₘₐₓ=446 nm (MeOH, ε=2.36×10⁴).

### Synthesis 27

### Cyanine Dye (154)

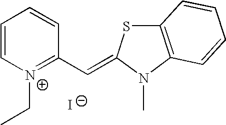

Using a method analogous to Method DD, with 2-(methylthio)benzothiazole methyl-p-toluenesulfonate (051) and 2-picoline ethiodide (083), the title compound was obtained. ¹H-NMR (500 MHz, d-DMSO): 1.46 (t, 3H, J=7.2, CH₃CH₂N); 3.77 (s, 3H, CH₃N); 4.53 (q, 2H, J=7.2, CH₃CH₂N); 5.86 (s, 1H, C═CH); 7.28 (t, 1H, J=7.1, Ar—H); 7.30 (t, 1H, J=8.0, Ar—H); 7.51 (t, 1H, J=8.4, Ar—H); 7.61 (d, 1H, J=8.3, Ar—H); 7.88 (d, 1H, J=7.9, Ar—H); 7.92 (d, 1H, J=8.8, Ar—H); 8.16 (t, 1H, J=7.9, Ar—H); 8.54 (d, 1H, J=6.5, Ar—H). ¹³C-NMR (125 MHz, d-DMSO): 13.79 (CH₃CH₂N); 33.02 (CH₃N); 51.57 (CH₃CH₂N); 81.63 (C═CH); 111.86, 117.49, 121.01, 122.36, 122.53, 123.61, 127.72, 140.81, 141.95, 143.73, 150.63, 157.77 (Arom. C). UV-vis λₘₐₓ=438 nm (MeOH, ε=3.37×10⁴).

### Synthesis 28

### Cyanine Dye (162A)

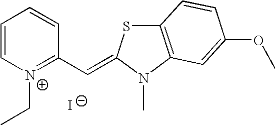

Using a method analogous to Method DD, with 2-(methylthio)-5-methoxybenzothiazole methyl-p-toluenesulfonate (121) and 2-picoline ethiodide (083), the title compound was obtained. ¹H-NMR (500 MHz, d-DMSO): 1.46 (t, 3H, J=7.2, CH₃CH₂N); 3.78 (s, 3H, OCH₃); 3.86 (s, 3H, CH₃N); 4.52 (q, 2H, J=7.2, CH₃CH₂N); 5.85 (s, 1H, C═CH); 6.92 (d, 1H, J=8.6, Ar—H); 7.22 (s, 1H, Ar—H); 7.26 (t, 1H, J=6.7, Ar—H); 7.76 (d, 1H, J=8.6, Ar—H); 7.88 (d, 1H, J=8.2, Ar—H); 8.14 (dd, 1H, J=8.2, 6.7, Ar—H); 8.52 (d, 1H, J=6.7, Ar—H).

### Synthesis 29

### Cyanine Dye (162B)

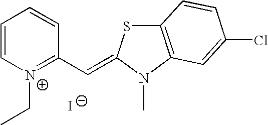

Using a method analogous to Method DD, with 2-(methylthio)-5-chlorobenzothiazole methyl-p-toluenesulfonate (136) and 2-picoline ethiodide (083), the title compound was obtained. ¹H-NMR (500 MHz, d-DMSO): 1.47 (t, 3H, J=7.2, CH₃CH₂N); 3.75 (s, 3H, CH₃N); 4.55 (q, 2H, J=7.2, CH₃CH₂N); 5.88 (s, 1H, C═CH); 7.32 (d, 1H, J=8.3, Ar—H); 7.35 (t, 1H, J=6.8 Ar—H); 7.73 (s, 1H, Ar—H); 7.87 (d, 1H, J=8.4, Ar—H); 7.93 (d, 1H, J=8.6, Ar—H); 8.20 (t, 1H, J=7.9, Ar—H); 8.60 (d, 1H, J=6.3, Ar—H). UV-vis λₘₐₓ=436 nm (MeOH, ε=3.57×10⁴).

### Synthesis 30

### Cyanine Dye (164)

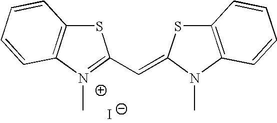

Using a method analogous to Method DD, with 2-(methylthio)benzothiazole methyl-p-toluenesulfonate (051) and 2-methylbenzothiazole methyl-p-toluenesulfonate (059), the title compound was obtained. ¹H-NMR (500 MHz, d-DMSO): 4.02 (s, 6H, CH₃N); 6.71 (s, 1H, C═CH); 7.50 (dd, 2H, J=8.4, 8.0, Ar—H); 7.68 (t, 2H, J=8.0, Ar—H); 7.87 (d, 2H, J=8.4, Ar—H); 8.21 (d, 2H, J=8.0, Ar—H). UV-vis λₘₐₓ=422 nm (MeOH, ε=6.59×10⁴).

### Synthesis 31

### (E)-N-Tolylcinnamamide (128)

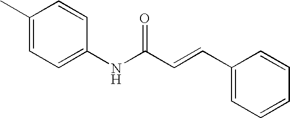

To a stirred solution of dimethylaminopyridine (DMAP) (220 mg, 1.80 mmol) and pyridine (1.46 mL, 18.0 mmol) in anhydrous dichloromethane (10 mL) under Ar at 0° C. was added a solution of cinnamoyl chloride (3.0 g, 18.0 mmol) dropwise over 10 minutes. The formation of a yellowish precipitate was observed. The mixture was stirred for 15 minutes at 0° C., and then a solution of p-toluidine (1.93 g, 18.0 mmol) in anhydrous dichloromethane (10 mL) was added dropwise over 10 minutes. The resultant yellow solution was stirred at 0° C. for 15 minutes, and then allowed to warm to room temperature and stirred for a further 1 hour. The solution was diluted with dichloromethane, washed with aqueous HCl (1 M, 3×100 mL), brine (20 mL), dried (MgSO₄), and concentrated to give the title compound as a white solid (3.68 g, 86%). ¹H-NMR (500 MHz, CDCl₃): 1.69 (br, 1H, NH); 2.34 (s, 3H, PhCH₃); 6.58 (d, 1H, J=15.5, ═CH); 7.15 (d, 2H, J=7.9, Ar—H); 7.33-7.39 (m, 3H, Ar—H); 7.46-7.56 (m, 3H, Ar—H); 7.60 (br, 1H, Ar—H); 7.56 (d, 1H, J=15.5, ═CH).

### Synthesis 32

### 6-Methylquinolin-2(1H)-one (137)

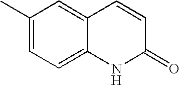

An intimate mixture of the (E)-N-tolylcinnamamide (128) (3.24 g, 13.67 mmol) and aluminium chloride (5.47 g, 41.0 mmol) was heated rapidly to melting, to give a dark brown/black viscous oil, which was heated at 10° C. for 1 hour. After the mixture was allowed to cool to room temperature, ice-water (60 g) was added, and the resultant precipitate was washed with water (30 mL), aqueous HCl (1 M, 2×40 mL), and then water (30 mL). The brownish solid (3.66 g) was dissolved in EtOAc (800 mL). Water (50 mL) was added, and the aqueous phase was extracted with EtOAc (2×50 mL). The combined organic extracts were washed with brine (15 mL) and evaporated to give the title compound as a brown solid (2.18 g, quant.) which was used without further purification. ¹H-NMR (500 MHz, CDCl₃): 2.42 (s, 3H, PhCH₃); 6.70 (d, 1H, J=15.5, ═CH); 7.31-7.37 (m, 3H, Ar—H); 7.76 (d, 1H, J=15.5, ═CH).

### Synthesis 33

### 2-Chloro-6-methylquinoline (140)

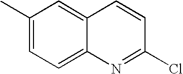

6-Methylquinolin-2(1H)-one (137) (2.18 g, 13.7 mmol) was suspended in POCl₃ (38 mL, 408 mmol) and stirred at 60° C. under Ar overnight. After cooling, the excess POCl₃ was removed by distillation, and then ice-water (150 mL) was added to the residue. Dichloromethane (150 mL) was added, and the aqueous phase was extracted with dichloromethane (50 mL). The combined organic extracts were washed with water (3×30 mL), brine (10 mL), dried (MgSO₄), and evaporated to give the title compound as a brown solid (2.183 g, 90%). ¹H-NMR (500 MHz, CDCl₃): 2.54 (3H, s, Ar—CH₃); 7.35 (d, 1H, J=8.6, Ar—H); 7.56-7.59 (m, 3H, Ar—H); 7.92 (d, 1H, J=8.5, Ar—H); 8.02 (d, 1H, J=8.6, Ar—H).

### Synthesis 34

### 2-Iodo-6-methylquinolinium ethiodide (146)

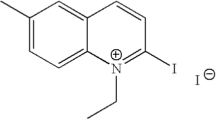

2-Chloro-6-methylquinoline (140) (1.0 g, 5.63 mmol) was suspended in iodoethane (5 mL, 62.5 mmol) and was stirred at reflux for 10 days. After cooling to room temperature, the mixture was filtered and the solid collected was washed with acetone (3×5 mL), to give the title compound as a yellow solid (498 mg, 21%). ¹H-NMR (500 MHz, d-DMSO): 1.19 (t, 3H, J=7.2, CH₂CH₃); 2.37 (s, 3H, Ar—CH₃); 4.25 (q, 2H, J=7.2, NCH₂); 6.56 (d, 1H, J=9.4, Ar—H); 7.44 (d, 1H, J=8.6, Ar—H); 7.48 (d, 1H, J=8.6, Ar—H); 7.51 (s, 1H, Ar—H); 7.82 (d, 1H, J=9.4, Ar—H).

### Synthesis 35

### Cyanine Dye (160)

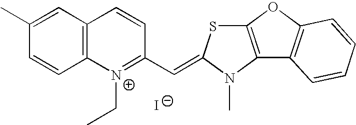

To a stirred solution of 2-iodo-6-methylquinolinium ethiodide (146) (334 mg, 0.79 mmol) and 2-methylbenzofuro-[3,2-d]thiazole methiodide (043) (215 mg, 0.65 mmol) in anhydrous DMF (7 mL) at room temperature was added anhydrous Et₃N (273 mL, 1.95 mmol). The formation of a purple/brown precipitate was observed. The mixture was stirred at room temperature for 1 hour, then acetone (130 mL) was added, and then the mixture was filtered. The solid collected was washed with acetone (50 mL), dried in a 90° C. oven, and cooled under vacuum to give the title compound as a red/brown solid (217 mg, 67%). ¹H-NMR (500 MHz, d-DMSO): 1.47 (t, 3H, J=7.1, CH₂CH₃); 2.43 (s, 3H, Ar—CH₃); 4.14 (s, 3H, NCH₃); 4.61 (q, 2H, J=7.1, NCH₂); 5.87 (s, 1H, C═CH); 7.48-7.51 (m, 2H, Ar—H); 7.67 (d, 1H, J=8.9, Ar—H); 7.71 (s, 1H, Ar—H); 7.75 (d, 1H, J=9.4, Ar—H); 7.83 (d, 1H, J=8.4, Ar—H); 7.90 (d, 1H, J=8.9, Ar—H); 8.15 (d, 1H, J=8.4, Ar—H); 8.21 (d, 1H, J=9.4, Ar—H). ¹³C-NMR (125 MHz, d-DMSO): 11.84 (CH₃CH₂N); 20.12 (CCH₃); 36.34 (CH₃N); 43.41 (CH₃CH₂N); 83.80 (C═CH); 112.96, 116.27, 116.90, 118.28, 118.36, 123.70, 124.44, 125.90, 128.78, 129.32, 134.66, 134.72, 136.91, 138.74, 140.82, 149.38, 159.34, 162.16 (Arom. C). ESI-MS (pos., MeOH): 373 (100, [M-I]⁻). HR-ESI-MS (pos.) (C₂₃H₂₁SON₂): 373.1374 (calc. 373.1375). UV-vis λₘₐₓ=504 nm (MeOH, ε=3.38×10⁴).

### Synthesis 36

### 4-Phenylthiazole-2-thiol (088)

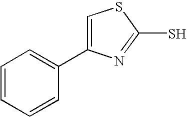

Method FF: To a solution of freshly prepared ammonium dithiocarbamate (460 mg, 4.18 mmol) in EtOH (45 mL) was added 2-bromoacetophenone (790 mg, 4.00 mmol) and the solution was stirred at reflux for 30 minutes. After allowing to cool to room temperature, the EtOH was evaporated and the residue dissolved in EtOAc (120 mL). Water (20 mL) was added, and the organic phase was washed with water (15 mL), brine (10 mL), and concentrated in vacuo to give the title compound as a white solid (800 mg, quant.). ¹H-NMR (500 MHz, CDCl₃): 6.76 (s, 1H, Ar—H); 7.35-7.45 (m, 5H, Ar—H); 10.52 (br, 1H, SH).

### Synthesis 37

### 2-(Methylthio)-4-phenylthiazole (093)

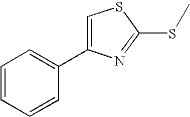

Using a method analogous to Method EE, with 4-phenylthiazole-2-thiol (088) (800 mg, 4.15 mmol), solid K₂CO₃ (686 mg, 4.97 mmol), and iodomethane (237 μL, 4.23 mmol), the title compound was obtained as a yellow liquid (750 mg, 87%). ¹H-NMR (500 MHz, CDCl₃): 2.79 (s, 3H, SCH₃); 7.25 (s, 1H, Ar—H); 7.35-7.49 (m, 5H, Ar—H).

### Synthesis 38

### 2-(Methylthio)-4-phenylthiazole methyl-p-toluenesulfonate (095)

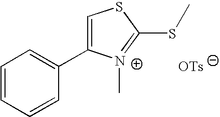

Using a method analogous to Method CC, with 2-(methylthio)-4-phenylthiazole (093) (750 mg, 3.62 mmol) and methyl p-toluenesulfonate (819 μL, 5.43 mmol), the title compound was obtained as a white solid (1.20 g, 84%). ¹H-NMR (500 MHz, d-DMSO): 2.24 (s, 3H, PhCH₃); 3.00 (s, 3H, SCH₃); 3.66 (s, 3H, NCH₃); 7.06 (d, 2H, J=7.9, Ar—H); 7.43 (d, 2H, J=7.9, Ar—H); 7.54-7.59 (m, 5H, Ar—H); 8.04 (s, 1H, Ar—H).

### Synthesis 39

### 4-(4-Methoxyphenyl)thiazole-2-thiol (099)

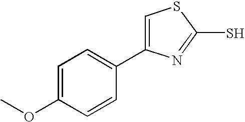

Using a method analogous to Method FF, with 4′-methoxy-2-bromoacetophenone (1.26 g, 5.5 mmol) and ammonium dithiocarbamate (640 mg, 5.82 mmol), the title compound was obtained as a white-grey solid (1.02 g, 83%). ¹H-NMR (500 MHz, CDCl₃): 3.82 (s, 3H, OCH₃); 6.57 (s, 1H, Ar—H); 6.98 (d, 2H, J=8.8, Ar—H); 7.45 (d, 2H, J=8.8, Ar—H); 11.11 (br, 1H, SH).

### Synthesis 40

### 4-(4-Methoxyphenyl)-2-(methylthio)thiazole (125)

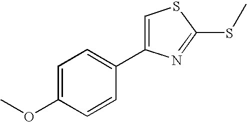

Using a method analogous to Method EE, with 4-(4-methoxyphenyl)thiazole-2-thiol (099) (1.02 g, 4.57 mmol), solid K₂CO₃ (760 mg, 5.49 mmol), and iodomethane (342 μL, 5.49 mmol), the title compound was obtained as a white crystalline solid (838 mg, 77%). ¹H-NMR (500 MHz, CDCl₃): 2.75 (s, 3H, SCH₃); 3.86 (s, 3H, OCH₃); 6.94 (d, 2H, J=8.5, Ar—H); 7.21 (s, 1H, Ar—H); 7.83 (d, 2H, J=8.5, Ar—H).

### Synthesis 41

### 4-(4-Methoxyphenyl)-2-(methylthio)thiazole methyl-p-toluenesulfonate (186B)

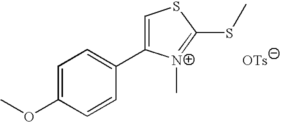

Using a method analogous to Method CC, with 4-(4-methoxyphenyl)-2-(methylthio)thiazole (125) (237 mg, 1.00 mmol) and methyl p-toluenesulfonate (225 μL, 1.50 mmol), the title compound was obtained as a white solid (422 mg, quant.). ¹H-NMR (500 MHz, d-DMSO): 2.23 (s, 3H, PhCH₃); 2.98 (s, 3H, SCH₃); 3.64 (s, 3H, NCH₃); 3.79 (s, 3H, OCH₃); 7.06 (d, 2H, J=8.0, Ar—H); 7.09 (d, 2H, J=8.8, Ar—H); 7.45 (d, 2H, J=8.0, Ar—H); 7.48 (d, 2H, J=8.8, Ar—H); 7.97 (s, 1H, Ar—H).

### Synthesis 42

### 4-(4-Chlorophenyl)thiazole-2-thiol (089)

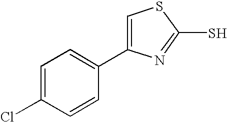

Using a method analogous to Method FF, with 4′-chloro-2-bromoacetophenone (934 mg, 4.0 mmol) and ammonium dithiocarbamate (460 mg, 4.18 mmol), the title compound was obtained as a white solid (900 mg, quant.). ¹H-NMR (500 MHz, CDCl₃): 6.72 (s, 1H, Ar—H); 7.38-7.47 (m, 4H, Ar—H); 10.99 (br, 1H, SH).

### Synthesis 43

### 4-(4-Chlorophenyl)-2-(methylthio)thiazole (094)

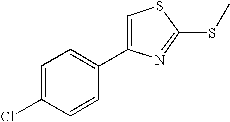

Using a method analogous to Method EE, with 4-(4-chlorophenyl)thiazole-2-thiol (089) (710 mg, 3.12 mmol), solid K₂CO₃ (517 mg, 3.74 mmol), and iodomethane (198 μL, 3.18 mmol), the title compound was obtained as a creamish solid (838 mg, 77%). ¹H-NMR (500 MHz, CDCl₃): 2.78 (s, 3H, SCH₃); 7.28 (s, 1H, Ar—H); 7.32-7.48 (m, 4H, Ar—H).

### Synthesis 44

### 4-(4-Chlorophenyl)-2-(methylthio)thiazole methyl-p-toluenesulfonate (096)

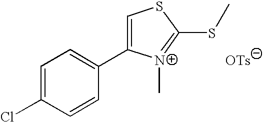

Using a method analogous to Method CC, with 4-(4-Chlorophenyl)-2-(methylthio)thiazole (094) (550 mg, 2.28 mmol) and methyl p-toluenesulfonate (515 μL, 3.42 mmol), the title compound was obtained as a white solid (850 mg, 87%). ¹H-NMR (500 MHz, d-DMSO): 2.24 (s, 3H, PhCH₃); 3.00 (s, 3H, SCH₃); 3.64 (s, 3H, NCH₃); 7.07 (d, 2H, J=7.9, Ar—H); 7.44 (d, 2H, J=7.9, Ar—H); 7.58 (d, 2H, J=8.5, Ar—H); 7.64 (d, 2H, J=8.5, Ar—H); 8.07 (s, 1H, Ar—H).

### Synthesis 45

### Cyanine Dye (180A1)

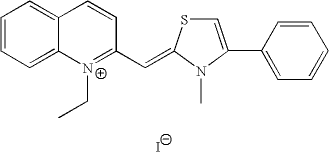

Using a method analogous to Method DD, with 2-(methylthio)-4-phenylthiazole methyl-p-toluenesulfonate (095) and quinaldine ethiodide (045), the title compound was obtained. ¹H-NMR (500 MHz, d-DMSO): 1.46 (d, 3H, J=7.0, CH₃CH₂N); 3.68 (s, 3H, CH₃N); 4.56 (br, 2H, CH₃CH₂N); 5.88 (s, 1H, C═CH); 7.09 (d, 1H, J=7.7, Ar—H); 7.43-7.52 (m, 2H, Ar—H); 7.56-7.62 (m, 4H, Ar—H); 7.80-7.86 (m, 2H, Ar—H); 7.91 (d, 1H, J=7.7, Ar—H); 7.95 (d, 1H, J=8.7, Ar—H); 8.21 (d, 1H, J=9.5, Ar—H). ¹³C-NMR (125 MHz, d-DMSO): 11.49 (CH₃CH₂N); 36.77 (CH₃N); 43.07 (CH₃CH₂N); 84.66 (C═CH); 107.63, 116.07, 118.21, 123.32, 124.59, 125.46, 127.95, 129.10, 129.40, 129.56, 130.21, 133.06, 138.38, 138.83, 144.58, 145.93, 150.09, 163.87 (Arom. C).

### Synthesis 46

### Cyanine Dye (180A2)

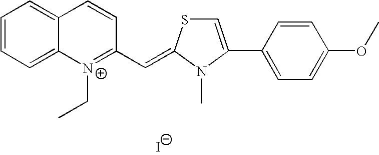

Using a method analogous to Method DD, with 4-(4-methoxyphenyl)-2-(methylthio)thiazole methyl-p-toluenesulfonate (186B) and quinaldine ethiodide (045), the title compound was obtained. ¹H-NMR (500 MHz, d-DMSO): 1.45 (d, 3H, J=6.7, CH₃CH₂N); 3.67 (s, 3H, CH₃N); 3.85 (s, 3H, OCH₃); 4.55 (br, 2H, CH₃CH₂N); 5.87 (s, 1H, C═CH); 7.15 (d, 2H, J=8.7, Ar—H); 7.38 (s, 1H, Ar—H); 7.48-7.53 (m, 3H, Ar—H); 7.79-7.84 (m, 2H, Ar—H); 7.90 (d, 1H, J=7.6, Ar—H); 7.94 (d, 1H, J=8.7, Ar—H); 8.20 (d, 1H, J=9.4, Ar—H). ¹³C-NMR (125 MHz, d-DMSO): 11.47 (CH₃CH₂N); 36.72 (CH₃N); 43.02 (CH₃CH₂N); 55.44 (OCH₃); 84.68 (C═CH); 107.01, 114.53, 116.02, 118.22, 121.07, 123.26, 124.52, 129.37, 131.10, 133.02, 138.24, 138.85, 144.55, 149.95, 160.59, 163.71 (Arom. C, missing 2 signals).

### Synthesis 47

### Cyanine Dye (180A3)

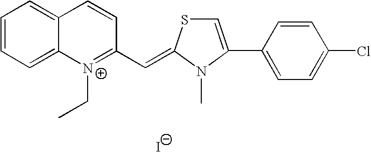

Using a method analogous to Method DD, with 4-(4-chlorophenyl)-2-(methylthio)thiazole methyl-p-toluenesulfonate (096) and quinaldine ethiodide (045), the title compound was obtained. ¹H-NMR (500 MHz, d-DMSO): 1.45 (d, 3H, J=6.7, CH₃CH₂N); 3.66 (s, 3H, CH₃N); 4.57 (br, 2H, CH₃CH₂N); 5.88 (s, 1H, C═CH); 7.47-7.49 (m, 1H, Ar—H); 7.51 (t, 1H, J=7.3, Ar—H); 7.62 (d, 2H, J=8.0, Ar—H); 7.68 (d, 2H, J=8.7, Ar—H); 7.82 (t, 2H, J=9.4, Ar—H); 7.92 (d, 1H, J=8.0, Ar—H); 7.96 (d, 1H, J=8.7, Ar—H); 8.22 (d, 1H, J=9.4, Ar—H). ¹³C-NMR (125 MHz, d-DMSO): 11.51 (CH₃CH₂N); 36.82 (CH₃N); 43.10 (CH₃CH₂N); 84.65 (C═CH); 108.09, 116.11, 118.19, 123.34, 124.64, 127.91, 129.16, 129.41, 131.48, 133.09, 135.12, 138.47, 138.82, 143.37, 150.16, 163.89 (Arom. C).

### Synthesis 48

### 3-Aminothiophene

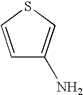

A mixture of NaOH (2M aq, 40 mL) and methyl-3-amino-2-thiophenecarboxylate (6.40 g, 40.7 mmol) was stirred at reflux for 30 minutes and then cooled to room temperature. Concentrated H₂SO₄ (8 mL) was added to the mixture until the pH was −1. The precipitate was filtered off, and pressed dry on filter paper to remove excess water. The yellow precipitate was dissolved in acetone (50 mL), dried (MgSO₄), and evaporated at 20° C. to give a yellow solid, which was treated with 1-propanol (12 mL) and anhydrous oxalic acid (4.0 g, 44.4 mmol). The suspension was stirred at 40° C. for 50 minutes, and then cooled to room temperature. To the mixture was added Et₂O (100 mL), and the precipitate was collected by filtration, to give the oxalate salt as a stable, white semi-crystalline solid (4.6 g, 60%). For characterisation, and immediately prior to use in subsequent reactions, the free base was prepared in the following manner. The oxalate salt (220 mg, 1.16 mmol) was suspended in H₂O (5 mL), and to this mixture was added NH₃ (30% aq., 0.2 mL) until the pH was >9. The solution was extracted with CH₂Cl₂ (3×5 mL). The combined organic extracts were washed with brine (5 mL), dried (MgSO₄), and evaporated to give the title compound as a brownish liquid (115 mg, quant. from oxalate salt); ¹H-NMR (500 MHz, CDCl₃): 3.60 (br, 2H, NH₂); 6.17 (dd, J=3.2, 1.6, 1H, Ar—H); 6.65 (dd, J=5.0, 1.6, 1H, Ar—H); 7.13 (dd, J=5.0, 3.2, 1H, Ar—H); ¹³C-NMR (125 MHz, CDCl₃): 99.24, 120.14, 124.29, 144.13 (Arom. C).

### Synthesis 49

### N-(Thiophen-3-yl)acetamide

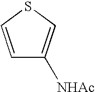

3-Aminothiophene (1.08, 10.9 mmol) was dissolved in Ac₂O (20 mL) and the mixture was stirred at room temperature for 3 hours. H₂O (50 mL) was added, and the mixture stirred for a further 30 minutes. NaOH (4M aq., 150 mL) was added until the pH was >9. The mixture was extracted with CH₂Cl₂ (3×100 mL). The combined organic extracts were washed with brine (30 mL), dried (MgSO₄), and evaporated. The product was crystallised from the residue using CH₂Cl₂/petroleum ether, to give the title compound as a white crystalline solid (994 mg, 65%); ¹H-NMR (500 MHz, CDCl₃): 2.17 (s, 3H, CH₃); 7.00 (dd, J=5.0, 1.3, 1H, Ar—H); 7.23 (dd, J=5.0, 3.2, 1H, Ar—H); 7.45 (br, 1H, N—H); 7.55 (dd, J=3.2, 1.3, 1H, Ar—H); ¹³C-NMR (125 MHz, CDCl₃): 18.64 (CH₃); 105.03, 115.64, 119.26, 130.27 (Arom. C); 162.11 (C═O).

### Synthesis 50

### N-(2-Thiocyanatothiophen-3-yl)acetamide

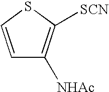

To a solution of N-(thiophen-3-yl)acetamide (1.03 g, 7.33 mmol) in MeOH (90 mL) was added solid NH₄SCN (2.23 g, 29.3 mmol). To this stirred solution at room temperature under argon was added dropwise over 15 minutes, Br₂ (414 μL, 8.07 mmol). The purple solution was stirred at room temperature for 30 minutes, and then H₂O (400 mL) was added. Solid NaOAc was added until the pH was −5, and then the solution was stirred for 3 hours. The solution was extracted with Et₂O (4×100 mL), and the combined organic extracts were washed with H₂O (2×100 mL), brine (30 mL), dried (MgSO₄), and evaporated. The product was crystallised from the residue with CHCl₃/petroleum ether, to give the title compound as yellowish rod-like crystals (990 mg, 68%); ¹H-NMR (500 MHz, CDCl₃): 2.30 (s, 3H, CH₃); 7.60 (d, J=6.0, 1H, Ar—H); 7.68 (br, 1H N—H); 7.96 (d, J=6.0, 1H, Ar—H); ¹³C-NMR (125 MHz, CDCl₃): 24.27 (CH₃); 109.21 (CN); 123.11, 132.70, 143.06, 167.20 (Arom. C).

### Synthesis 51

### S-3-Acetamidothiophen-2-yl ethanethioate

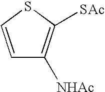

To a solution of Na₂S.9H₂O (2.00 g, 8.30 mmol) in H₂O (25 mL) was added gradually N-(2-thiocyanatothiophen-3-yl)acetamide (950 mg, 4.80 mmol) and the resultant yellow solution was stirred at room temperature for 2 hours. The solution was filtered and cooled to 0° C., and Ac₂O (4 mL, excess) was added. The formation of a precipitate was observed. The mixture was stirred at 0° C. for 30 minutes, and then filtered, and the solid collected was washed with H₂O (2×5 mL), to give the title compound as yellowish crystalline solid (1.03 g, quant.); ¹H-NMR (500 MHz, CDCl₃): 2.19 (s, 3H, CH₃); 2.46 (s, 3H, CH₃); 7.42 (br, 1H N—H); 7.54 (d, J=5.7, 1H, Ar—H); 7.97 (d, J=5.7, 1H, Ar—H); ¹³C-NMR (125 MHz, CDCl₃): 24.21 (CH₃); 29.66 (CH₃); 122.96, 130.53, 141.23, 167.05 (Arom. C).

### Synthesis 52

### 2-Methylthieno[3,2-d]thiazole

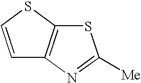

To a solution of S-3-acetamidothiophen-2-yl ethanethioate (570 mg, 2.65 mmol) in EtOH (30 mL) was added TsOH.H₂O (306 mg, 1.16 mmol), and the solution was stirred at reflux for 2 hours, and then cooled to room temperature. The solution was concentrated in vacuo, and NaHCO₃ (sat. aq., 100 mL) was added to the residue. The mixture was extracted with CH₂Cl₂ (2×40 mL). The combined organic extracts were washed with H₂O (20 mL), brine (5 mL), dried (MgSO₄), and evaporated to give a brown oil. Flash chromatography (eluent: petroleum ether/Et₂O, 10:1) yielded the title compound as an orange liquid (212 mg, 52%); ¹H-NMR (500 MHz, CDCl₃): 2.83 (s, 3H, CH₃); 7.40 (s, 2H, Ar—H); ¹³C-NMR (125 MHz, CDCl₃): 20.25 (CH₃); 118.57, 127.91, 129.43, 160.63, 169.10 (Arom. C).

### Synthesis 53

### 2-Methylthieno[3,2-d]thiazole methiodide (062)

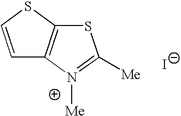

Reaction of 2-methylthieno[3,2-d]thiazole (200 mg, 1.29 mmol) and iodomethane (240 μL, 3.87 mmol) in acetone at reflux for 24 hours gave the title compound as a brownish solid (300 mg, 78%); ¹H-NMR (500 MHz, d-DMSO): 3.02 (s, 3H, CCH₃); 4.16 (s, 3H, NCH₃); 7.77 (d, J=5.7, 1H, Ar—H); 8.10 (d, J=5.7, 1H, Ar—H); ¹³C-NMR (125 MHz, d-DMSO): 16.66 (CCH₃); 37.62 (NCH₃); 114.14, 128.65, 135.73, 148.37 (Arom. C).

### Synthesis 54

### Cyanine Dye (MMR218)

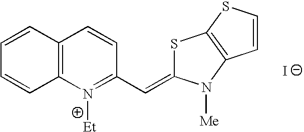

The cyanine dye (MMR218) was synthesised from 2-methylthieno[3,2-d]thiazole methiodide (062) and 2-iodoquinoline ethiodide (009) in a manner analogous to the preparation of the compound (027); ¹H-NMR (500 MHz, d-DMSO): 1.46 (t, 3H, J=7.1, CH₃CH₂N); 4.00 (s, 3H, NCH₃); 4.57 (q, J=7.2, 2H, CH₃CH₂N); 5.87 (s, 1H, C═CH); 7.51 (t, J=7.4, 1H, Ar—H); 7.54 (d, J=5.5, 1H, Ar—H); 7.81 (d, J=9.5, 1H, Ar—H); 7.82 (t, J=7.4, 1H, Ar—H); 7.88 (d, J=5.5, 1H, Ar—H); 7.91 (d, J=7.4, 1H, Ar—H); 7.95 (d, J=9.2, 1H, Ar—H); 8.24 (d, J=9.2, 1H, Ar—H); ¹³C-NMR (125 MHz, d-DMSO): 11.59 (CH₃CH₂N); 36.15 (CH₃N); 43.19 (CH₃CH₂N); 84.81 (C═CH); 113.56, 116.14, 118.35, 118.56, 123.48, 124.72, 129.38, 132.83, 133.12, 138.48, 146.49, 150.10, 162.26, 167.25 (Arom. C); UV-vis λₘₐₓ=484 nm (MeOH); ESI-MS (pos., MeOH): 325 (100, [M-I]⁺). HR-ESI-MS (pos.) (C₁₈H₁₇S₂N₂): 325.0836 (calc. 325.0833).

### Synthesis 55

### 1-Ethyl-2-fluoropyridinium tetrafluoroborate

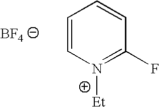

A solution of 2-fluoropyridine (3.00 mL, 3.39 g, 34.92 mmol) in 1,2-dichloroethane (10 mL) was added dropwise to a solution of triethyloxonium tetrafluoroborate (6.63 g, 34.92 mmol) in 1,2-dichloroethane (30 mL) and the resulting solution was heated at reflux for 24 hours. The resulting solution was concentrated and the residue taken up in dichloromethane (30 mL) and hexane (30 mL) and the resulting solid filtered to give the title compound as a white solid (1.94 g, 26%); 5H (500 MHz; d₆-Acetone) 1.70 (3H, t, J 7.3, CH₃), 4.84 (2H, qd, J 7.3 & 2.5, CH₂), 8.07 (2H, m, Ar—H), 8.82-8.87 (1H, m, Ar—H), 8.98 (1H, m, Ar—H).

### Synthesis 56

### Cyanine Dye (MMR307)

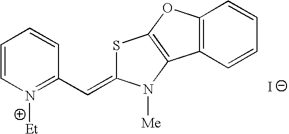

A suspension of 1-ethyl-2-fluoropyridinium tetrafluoroborate (0.213 g, 1.00 mmol) and 1,2-dimethyl-benzo[4,5]furo[3,2-d]thiazol-1-ium iodide (0.331 g, 1.00 mmol) in acetonitrile (5 mL) was stirred for five minutes before addition of N,N-diisopropylethylamine (0.35 mL, 0.26 g, 2.00 mmol). The resulting solution/suspension was stirred for 2 hours, acetone (15 mL) added, and the reaction filtered to give the title compound as a red solid (0.120 g, 28%); δ_{H} (500 MHz; d₆-DMSO) 1.45 (3H, t, J 7.2, CH₂CH₃), 4.00 (3H, s, NCH₃), 4.47 (2H, q, J 7.2, CH₂CH₃), 5.70 (1H, s, HC═C), 7.13 (1H, td, J 7.0 & 1.1, Ar—H), 7.46 (2H, m, Ar—H), 7.64 (1H, d, J 8.7, Ar—H), 7.79 (1H, m, Ar—H), 8.05-8.11 (2H, m, Ar—H), 8.44 (1H, d, J 5.9, Ar—H); 5c(125 MHz; d₆-DMSO) 13.55 (NCH₂CH₃), 35.52 (NCH₃), 51.13 (NCH₂CH₃), 80.36 (HC═C), 112.81 (Ar—CH), 115.85 (Ar—CH), 117.03 (quaternary C), 118.01 (Ar—CH), 120.24 (Ar—CH), 124.19 (Ar—CH), 125.35 (Ar—CH), 128.53 (quaternary C), 138.58 (quaternary C), 141.33 (Ar—CH), 143.34 (Ar—CH), 148.99 (quaternary C), 159.14 (quaternary C), 159.64 (quaternary C). Found [M-I]+309.1059, C₁₈H₁₇N₂OS requires 309.1056.

### Synthesis 57

### N-(4-Methoxy-phenyl)-3-phenyl-acrylamide

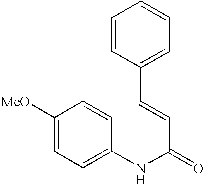

A solution of cinnamoyl chloride (4.58 g, 27.49 mmol) in dichloromethane (30 mL) was added dropwise to a solution of 4-N,N-dimethylaminopyridine (0.37 g, 3.05 mmol) and pyridine (3.20 mL, 3.14 g, 39.70 mmol) in dichloromethane (100 mL) at 0° C. After 15 minutes, a solution of p-anisidine (3.76 g, 30.54 mmol) in dichloromethane (30 mL) was added and the resulting solution stirred for 48 hours. After this time, the reaction mixture was washed with dilute aqueous hydrochloric acid (3×100 mL), dried (MgSO₄), filtered, and the solvent removed under reduced pressure to give the title compound as a colourless solid (6.64 g, 95%); δ_{H} (500 MHz; CDCl₃) 3.75 (3H, s, OCH₃), 6.69 (1H, d, J 15.5, HC═CHCO), 6.83 (2H, d, J 8.8, Ar—H), 7.28-7.32 (3H, m, Ar—H), 7.42 (2H, m, Ar—H), 7.58 (1H, d, J 8.8, Ar—H), 7.73 (1H, d, J 15.5, HC═CHCO); 5c(125 MHz; CDCl₃) δ5.36 (OCH₃), 114.12 (Ar—C—H), 121.22 (Ar—C—H), 122.08 (Ar—C—H), 127.83 (Ar—C—H), 128.70 (Ar—C—H), 129.66 (Ar—C—H), 131.29 (quaternary C), 134.68 (Ar—C—H), 141.62 (quaternary C), 156.45 (quaternary C), 164.38 (quaternary C). Found [M+Na]⁺ 276.09939, C₁₆H₁₅NaNO₂ requires 276.09950.

### Synthesis 58

### N-Ethyl-N-(4-methoxy-phenyl)-3-phenyl-acrylamide

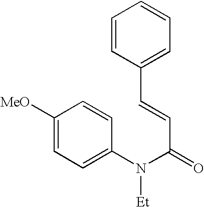

Sodium hydride (1.07 g of a 60% dispersion in mineral oil, 26.82 mmol) was added portionwise to a solution of N-(4-methoxy-phenyl)-3-phenyl-acrylamide (6.47 g, 25.54 mmol) in DMF (100 mL) at room temperature and the resulting mixture stirred for 1 hour, cooled to 0° C. and ethyl iodide (6.20 mL, 12.00 g, 76.70 mmol) added. Stirring was continued for 24 hours and saturated aqueous ammonium chloride (50 mL) added. Solvent was removed under reduced pressure and the residue partitioned between dichloromethane (200 mL) and water (200 mL). The aqueous phase was extracted with dichloromethane (2×200 mL) and the combined extracts were dried (MgSO₄), filtered, and the solvent removed under reduced pressure. Purification by flash column chromatography (2:1, petroleum spirit: ethyl acetate) gave the title compound as a colourless oil (7.00 g, 97%); δ_{H} (500 MHz; CDCl₃) 1.16 (3H, t, J 6.6, NCH₂CH₃), 3.84 (5H, m, OCH₃ & NCH₂CH₃), 6.29 (1H, d, J 15.5, HC═CHCO), 6.94 (2H, d, J 7.7, Ar—H), 7.11 (2H, d, J 7.7, Ar—H), 7.27 (5H, m, Ar—H), 7.77 (1H, d, J 15.5, HC═CHCO); 5c(125 MHz; CDCl₃) 12.86 (NCH₂CH₃), 44.27 (NCH₂CH₃), 55.33 (OCH₃), 114.57 (Ar—C—H), 119.11 (Ar—C—H), 127.62 (Ar—C—H), 128.48 (Ar—C—H), 129.22 (Ar—C—H), 129.34 (Ar—C—H), 134.44 (quaternary C), 135.17 (quaternary C), 141.30 (Ar—C—H), 158.79 (quaternary C), 165.66 (quaternary C).

### Synthesis 59

### 1-Ethyl-6-hydroxyquinolin-2(1H)-one

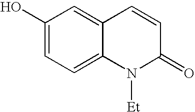

Aluminium chloride (13.27 g, 99.52 mmol) was added in one portion to a solution of N-ethyl-N-(4-methoxy-phenyl)-3-phenyl-acrylamide (7.00 g, 24.88 mmol) in chlorobenzene (100 mL) and the resulting mixture rapidly heated to 120° C. and maintained at this temperature for 2 hours. The mixture was cooled and poured onto crushed ice water (100 mL) and stirred for 1 hour, concentrated (−50%) and filtered. The crude compound thus obtained was purified by column chromatography through a plug of silica (1:1 petroleum spirit:ethyl acetate→ethyl acetate) to give the title compound as a colourless solid that was purified by recrystallisation (MeOH) to give colourless crystals (3.53 g, 75%); δ_{H} (500 MHz; d₆-DMSO) 1.18 (3H, t, J 6.9, NCH₂CH₃), 4.21 (2H, q, J 6.9, NCH₂CH₃), 6.53 (1H, d, J 9.5, Ar—H), 7.04 (1H, d, J 2.6, Ar—H), 7.10 (1H, dd, J 8.9 & 2.6, Ar—H), 7.39 (1H, d, J 8.9, Ar—H), 7.75 (1H, d, J 9.5, Ar—H), 9.46 (1H, br s, OH); 5c(125 MHz; d₆-DMSO) 12.90 (NCH₂CH₃), 36.53 (NCH₂CH₃), 112.99 (Ar—C—H), 115.62 (Ar—C—H), 119.87 (Ar—C—H), 121.50 (Ar—C—H), 121.52 (quaternary C), 132.13 (quaternary C), 138.73 (Ar—C—H), 152.15 (quaternary C), 160.27 (quaternary C). Found [M+Na]⁺ 212.06816, C₁₁H₁₁NaNO₂ requires 212.06820.

### Synthesis 60

### 1-Ethyl-6-methoxyquinolin-2(1H)-one

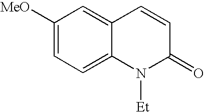

Methyl iodide (1.10 mL, 2.45 g, 17.28 mmol) was added to a mixture of 1-ethyl-6-hydroxyquinolin-2(1H)-one (0.818 g, 4.32 mmol) and potassium carbonate (1.49 g, 10.81 mmol) in DMF (15 mL) and the resulting mixture stirred for 4 hours. Solvent and excess methyl iodide were removed under reduced pressure and the residue partitioned between dichloromethane (50 mL) and water (50 mL). The aqueous phase was extracted with dichloromethane (2×50 mL) and the combined extracts were dried (MgSO₄), filtered, and the solvent removed under reduced pressure. Purification by flash column chromatography (1:1. petroleum spirit:ethyl acetate) gave the title compound as a colourless solid (0.829 g, 94%); δ_{H} (500 MHz; CDCl₃) 1.34 (3H, t, J 6.6, NCH₂CH₃), 3.85 (3H, s, OCH₃), 4.33 (2H, q, J 6.6, NCH₂CH₃), 6.69 (1H, d, J 9.5, Ar—H), 6.99 (1H, d, J 3.2, Ar—H), 7.17 (1H, dd, J 9.5 & 3.2, Ar—H), 7.30 (1H, d, J 9.5, Ar—H), 7.58 (1H, d, J 9.5, Ar—H); δ_{C} (125 MHz; CDCl₃) 12.78 (NCH₂CH₃), 37.28 (NCH₂CH₃), 55.64 (OCH₃), 110.61 (Ar—C—H), 115.26 (Ar—C—H), 119.23 (Ar—C—H), 121.66 (quaternary C), 122.39 (Ar—C—H), 133.52 (quaternary C), 138.28 (Ar—C—H), 154.45 (quaternary C), 161.35 (quaternary C). Found [M+H]⁺ 204.10187, C₁₂H₁₄NO₂ requires 204.10187.

### Synthesis 61

### 2-Chloro-1-ethyl-6-methoxyquinolinium iodide

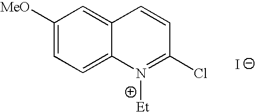

A suspension of 1-ethyl-6-methoxyquinolin-2(1H)-one (0.79 g, 3.88 mmol) in phosphorus oxychloride (3.60 mL, 5.94 g, 38.77 mmol) was heated at 120° C. for 24 hours, cooled to room temperature and evaporated to dryness under reduced pressure. The dark residue was taken up in water (10 mL) and washed with toluene (50 mL). Saturated aqueous potassium iodide was added (˜30 mL), whereupon the product precipitated and was collected by filtration to give the title compound as a yellow solid (0.41 g, 30%); δ_{H} (500 MHz; d₆-DMSO) 1.55 (3H, t, J 7.3, NCH₂CH₃), 4.00 (3H, s, OCH₃), 5.16 (2H, q, J 7.3, NCH₂CH₃), 7.90 (1H, dd, J 9.8 & 2.8, Ar—H), 7.93 (1H, m, Ar—H), 8.31 (1H, d, J 8.8, Ar—H), 8.59 (1H, d, J 9.8, Ar—H), 9.07 (1H, d, J 8.8, Ar—H).

### Synthesis 62

### Cyanine Dye (MMR216)

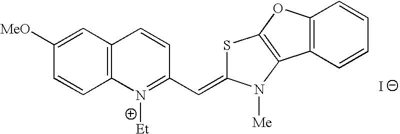

A suspension of 2-chloro-1-ethyl-6-methoxyquinolinium iodide (0.18 g, 0.42 mmol) and 1,2-dimethyl-benzo[4,5]furo[3,2-d]thiazol-1-ium iodide (0.17 g, 0.38 mmol) in DMF (10 mL) was stirred for five minutes before addition of N,N-diisopropylethylamine (0.14 mL, 0.11 g, 0.83 mmol). The resulting solution/suspension was stirred for 2 hours, acetone (15 mL) added, and the reaction filtered to give the title compound as a red solid (0.16 g, 28%); δ_{H} (500 MHz; d₆-DMSO) 1.47 (3H, t, J 7.1, NCH₂CH₃), 3.88 (3H, s, NCH₃), 4.11 (3H, s, OCH₃), 4.62 (2H, q, J 7.1, NCH₂CH₃), 5.85 (1H, s, HC═C), 7.45-7.50 (4H, m, Ar—H), 7.80-7.83 (2H, m, Ar—H), 7.97 (1H, d, J 9.1, Ar—H), 8.13 (1H, m, Ar—H), 8.25 (1H, d, J 9.1, Ar—H). Found [M-I]+389.1316, C₂₃H₂₁N₂O₂S requires 389.1318.

### Biological Methods

### In Vitro—Modified MTT Assay

Relative drug sensitivities (Resistance Factor) and IC₅₀ values were determined by a modified MTT assay (see, e.g., Mosmann, 1983; Plumb et al., 1989), as described below:
(1) Trypsinise a sub-confluent monolayer culture and collect cells in growth medium containing serum. Centrifuge the suspension (200 g, 5 minutes) to pellet cells, re-suspend in growth medium, and count cells. Dilute cells to a density of 5×10³ cells/mL allowing 60 mL of cell suspension for 3 microtitre plates. Transfer cell suspension to a 10 cm petri dish, and with a multichannel pipette add 200 μL to each well of the central 10 columns of a flat bottomed 96-well plate (80 wells per plate) starting with column 2 and ending with column 11. Add 200 μL of growth medium to the 8 wells in columns 1 and 12. Put the plates in a plastic box and incubate in a humidified atmosphere at 37° C. for 2-3 days so that cells are in the exponential phase of growth for drug addition.
(2) Prepare a serial 5-fold dilution of the cytotoxic drug in growth medium to give 8 concentrations. Pipette 6 mL of growth medium in to each of 7 universal containers (30 mL). Prepare 10 mL of the highest concentration of the drug and transfer 1.5 mL of this solution to the first universal container. Mix and then transfer 1.5 mL to the next universal container. Continue until the seventh universal container is reached. The concentrations should be chosen so that the highest concentration kills most of the cells and the lowest kills none of the cells. Normally, three plates are used for each drug to give triplicate determinations within one experiment.
(3) The medium is removed from all the wells in columns 2 to 11. This can be achieved with a hypodermic needle attached to a suction line. Cells in the 8 wells in columns 2 and 11 are fed with 200 μL of fresh growth medium. The drug solutions are transferred to 10 cm petri dishes and 200 μL added to all 8 wells of a column. For ease of analysis arrange the drug solutions in order so that the highest is in column 3 down to the lowest in column 10. Plates are returned to the plastic box and incubated for 24 hours.
(4) At the end of the drug exposure period, the medium is removed from all wells containing cells and the cells are fed with 200 μL of fresh medium.
(5) The plates are fed daily for two more days.
(6) The plates are fed with 200 μL of fresh medium and 50 μL of the MTT solution (5 mg/mL in PBS) is added to all wells in columns 1 to 11. Plates are wrapped in aluminium foil and incubated for 4 hours in a humidified atmosphere at 37° C.
(7) The medium and MTT are then removed from the wells and the purple MTT-formazan crystals dissolved by addition of 200 μL of DMSO to all wells in columns 1 to 11. Glycine buffer is added (25 μL per well) to all wells containing DMSO.
(8) Absorbance is recorded at 570 nm with the wells in column 1, which contained medium and MTT but no cells used as a blank.
(9) A graph is plotted of absorbance (y-axis) against drug concentration (x-axis). The mean absorbance reading from the wells in columns 2 and 11 is used as the control absorbance and the IC₅₀ concentration is determined as the drug concentration required to reduce the absorbance to half that of the control. IC₁₀ or IC₉₀ values can be determined in the same manner.

Cell lines used for the MTT assays are:
(1) Cisplatin resistant ovarian cell lines A2780/cp70 (deficient in MMR with epigenetically silenced MLH1 gene) and A2780/mcp1 (deficient in MMR with epigenetically silenced MLH1 gene) and the matched parental A2780 line (proficient in MMR with expressed MLH1 gene) (see, e.g., Strathdee et al., 1999; Anthoney et al., 1996).
(2) A2780/cp70 with chromosome 3 reintroduced which either express MLH1 (A1) or do not express MLH1 (A2) (see, e.g., Durant et al., 1999).
(3) A panel of colon lines that have different MMR status.

### In Vivo—Xenograft Studies

For xenograft experiments, tumour cells were injected into the flanks of athymic nude mice and allowed to establish until palpable tumour could be detected. Following single bolus injection of drug, tumour growth was monitored using calliper measurement of tumour size and calculation of tumour volume.

Cells in exponential phase of growth were harvested with trypsin and re-suspended in phosphate buffered saline. About 10⁷ cells in a total volume of 200 μL of PBS were injected subcutaneously in the right flank of CD1 nude mice. They were used for experiments after about 7 to 10 days by which time the mean tumour diameter was about 0.5 cm.

Mice were weighed and tumour volumes measured. The mice were randomised in groups of 6 and treated either with the drug or with the solvent alone. The drug was dissolved in DMSO and diluted in sterile PBS just before injection to give a final DMSO concentration of 1%. The drugs solution was prepared such that the injection volume was 5 μL per gram body weight. Cages were placed on a heated surface (70° C.) for 15 minutes in order to dilate the tail veins of the mice. The mice were restrained and drug was administered through a 26-gauge needle as a single intravenous bolus dose via a tail vein.

Mice were then weighed daily and tumour volumes estimated from two measurements of the diameter taken at a 90° angle. The volume was determined by the formula: v=D³/6×π, where v=volume and D is the mean diameter. Results (Mean and SEM) are expressed as the relative tumour volume, which is the tumour volume on any given day divided by the initial tumour volume calculated for each mouse.

### Biological Data

A2780/cp70 and A2780/mcp1 cell lines are cisplatin resistant derivatives of the human ovarian tumour cell line A2780. A2780/cp70 and A2780/mcp1 have lost expression of MLH1, have lost MMR activity, and have microsatellite instability compared to A2780. The CpG island at the MLH1 gene is methylated in A2780/cp70 and A2780/mcp1, but not A2780.

IC₅₀ values (μM) for A2780, A2780/cp70, and A2780/mcp1 and Resistance Factors (RF) (the fold difference in resistance from parental A2780) were determined based on the MTT assay described above for several compounds of the present invention (including, e.g., MMR201). Compounds showing increased activity against cisplatin MMR deficient cells have RF values less than 1.

Compound MMR201 and analogues show increased growth inhibitory activity against cisplatin resistant A2780 derivatives that have lost MMR due to epigenetic silencing of MLH1.

Data for the reference compound, Cisplatin, and several compounds of the present invention (including, e.g., MMR201) are shown in the table below.

TABLE 1
IC₅₀ and RF for a 24 hour drug exposure

| | A2780 | A2780/cp70 | | A2780/mcp1 | |
|---|---|---|---|---|---|
| Compound | IC50 (μM) | IC50 (μM) | RF | IC50 (μM) | RF |
| Cisplatin | 0.215 ± 0.037 | 1.74 ± 0.07 | 8.1 | 0.547 ± 0.076 | 2.5 |
| | 0.244 ± 0.075 | 1.68 ± 0.14 | 6.9 | 0.672 ± 0.111 | 2.8 |
| MMR201 | 0.058 ± 0.005 | 0.017 ± 0.001 | 0.29 | 0.014 ± 0.001 | 0.24 |
| MMR211 | 0.069 ± 0.007 | 0.040 ± 0.004 | 0.58 | 0.038 ± 0.006 | 0.55 |
| MMR212 | 1.67 ± 0.37 | 0.435 ± 0.022 | 0.26 | 0.481 ± 0.048 | 0.29 |
| MMR213 | 1.23 ± 0.051 | 0.325 ± 0.044 | 0.26 | 0.358 ± 0.103 | 0.29 |
| MMR302 | 13.59 ± 1.55 | 7.10 ± 0.37 | 0.52 | 12.94 ± 1.00 | 0.95 |
| MMR304 | 64.50 ± 3.91 | 18.02 ± 1.30 | 0.28 | 35.62 ± 1.96 | 0.55 |
| MMR307 | 0.752 ± 0.073 | 0.932 ± 0.095 | 1.24 | 0.716 ± 0.041 | 0.95 |

A1, E1, and A2 are derived by transfer of human chromosome 3 into A2780/cp70. MLH1 is located on chromosome 3. The A1 and E1 line re-expresses MLH1 (and is MMR proficient), while the A2 line does not express MLH1 (and remains mismatch deficient).

IC₅₀ values (μM) for A1 (MLH1+ve, MMR proficient) or E1 (MLH1+ve, MMR proficient) and A2 (MLH1−ve, MMR deficient) lines and Resistance Factors (RF) (the fold difference in resistance between the A2 line, MMR deficient, and the A1 line, MMR proficient) were determined based on the MTT assay described above for several compounds of the present invention (including, e.g., MMR201). Compounds showing increased activity against the MLH1 deficient A2 line compared to the MLH1 proficient A1 or E1 line have RF values less than 1.

Compound MMR201 and analogues show increased growth inhibitory activity against A27801 cp70 chromosome 3 transferrants that do not express MLH1 compared to a matched line which does express MLH1.

Data for the reference compound, Cisplatin, and several compounds of the present invention (including, e.g., MMR201) are shown in the table below.

TABLE 2

| | | A1 or E1 | A2 | |
|---|---|---|---|---|
| | | MLH1 +ve | MLH1 −ve | |
| | A1 or | MMR proficient | MMR deficient | |
| Compound | E1 | IC50 (μM) | IC50 (μM) | RF |
| Cisplatin | A1 | 0.715 ± 0.069 | 1.905 ± 0.087 | 2.7 |
| MMR201 | A1 | 0.121 ± 0.018 | 0.023 ± 0.001 | 0.19 |
| MMR211 | A1 | 0.144 ± 0.009 | 0.027 ± 0.003 | 0.19 |
| MMR212 | A1 | 4.10 ± 0.45 | 0.681 ± 0.076 | 0.17 |
| MMR213 | A1 | 2.55 ± 0.18 | 0.530 ± 0.018 | 0.21 |
| MMR302 | A1 | 2.83 ± 0.46 | 4.15 ± 0.80 | 1.5 |
| MMR304 | A1 | 54.17 ± 10.84 | 16.11 ± 2.54 | 0.30 |
| MMR307 | E1 | 0.937 ± 0.066 | 0.648 ± 0.085 | 0.69 |

The table below shows additional data for MMR201 in regard to a panel of colon tumour cell lines of differing MMR status. CACO₂, COLO320DM, HT29 and T84 are proficient for MMR, while HCT116, SW48, DLD1, HCT15 and LOVO are deficient for MMR. The lines most sensitive to MMR201 are the cell lines that are deficient in MLH1 (HCT116 and SW48).

TABLE 3

| | | | |
|---|---|---|---|
| | | Cisplatin | MMR201 |
| Colon cell line | p53 | IC50 (μM) | IC50 (nM) |

MMR proficient

| | | | |
|---|---|---|---|
| CACO2 | mut | 1.83 ± 0.20 | 473.6 ± 16.0 |
| COLO320DM | mut | 2.90 ± 0.29 | 593.8 ± 28.5 |
| HT29 | mut | 5.67 ± 0.64 | 118.1 ± 9.1 |
| T84 | ? | 4.23 ± 0.86 | 1474 ± 175 |

MMR deficient

| | | | |
|---|---|---|---|
| HCT116 (MLH1) | wt | 2.13 ± 0.03 | 37.9 ± 3.1 |
| SW48 (MLH1) | wt | 1.82 ± 0.07 | 76.9 ± 7.3 |
| RKO (MLH1) | | 4.01 ± 0.10 | 27.2 ± 0.7 |
| DLD1 (MSH6) | mut | 1.67 ± 0.03 | 186.0 ± 8.3 |
| HCT15 (MSH6) | wt/mut | 2.92 ± 0.02 | 583.8 ± 55.1 |
| LOVO (MSH2) | wt | 1.83 ± 0.09 | 430.9 ± 11.2 |

Unknown

| | | | |
|---|---|---|---|
| BE | ? | 8.67 ± 1.42 | 125.3 ± 17.3 |

Additional data for the reference compound, Cisplatin, and MMR201 in a range of cancer cell lines are shown in the table below. (Non-small cell lung cancer is denoted NSCLC.)

TABLE 4

| | Cell line | Cisplatin (μM) | MMR201 (nM) |
|---|---|---|---|
| Ovary | OVCAR3 | 0.239 ± 0.020 | 252.8 ± 22.0 |
| | OVCAR5 | 1.78 ± 0.10 | 699.4 ± 54.6 |
| | OVCAR4 | 1.64 ± 0.15 | 733.1 ± 41.2 |
| Breast | MDAMB231 | 11.2 ± 0.7 | 234.6 ± 22.5 |
| | T47D | 5.42 ± 0.22 | 319.3 ± 26.0 |
| | MCF7 | 1.78 ± 0.17 | 869.6 ± 54.8 |
| NSCLC | LDAN | 1.23 ± 0.07 | 114.4 ± 0.07 |
| | A549 | 1.13 ± 0.04 | 119.1 ± 10.6 |
| | H125 | 3.69 ± 0.43 | 129.5 ± 15.0 |
| | WIL | 3.94 ± 0.22 | 152.9 ± 13.7 |
| | CALU | 7.25 ± 0.58 | 379.8 ± 7.5 |
| | SKMES | 5.16 ± 0.08 | 398.0 ± 61.9 |
| Melanoma | HS852 | 1.60 ± 0.09 | 75.3 ± 7.1 |
| | HS294 | 1.49 ± 0.08 | 104.8 ± 5.1 |
| | A375 | 0.613 ± 0.051 | 117.8 ± 17.7 |
| | C8161 | 3.46 ± 0.16 | 254.2 ± 0.4 |
| Prostate | DU145 | 1.68 ± 0.04 | 328.0 ± 7.8 |
| | PC3 | 2.27 ± 0.27 | 530.9 ± 50.3 |

The efficacy of MMR201 to inhibit growth of human tumour cells grown as a xenograft in nude mice has also been examined.

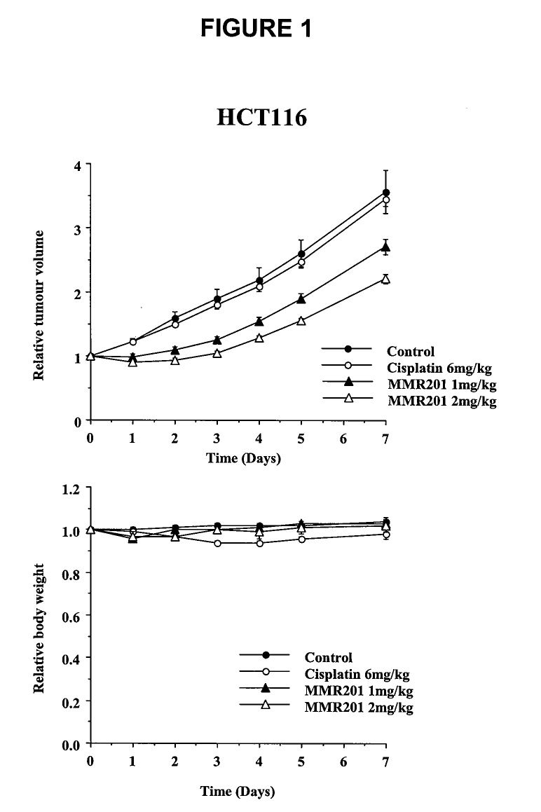

FIG. 1 shows two graphs, one of relative tumour volume versus time (days), and one of relative body weight versus time (days), for HCT116 human colon tumour cell line grown as xenograft in nude mice. Mice were treated with a single bolus injection on day 0 with (a) control, (b) cisplatin (6 mg/kg) i.p., (c) MMR201 (1 mg/kg) i.v., and (d) MMR201 (2 mg/kg) i.v. Error bars represent standard error of the mean.

These data show that MMR201 inhibits growth of MLH1 deficient HCT116 colon cells at tolerated doses in mice.

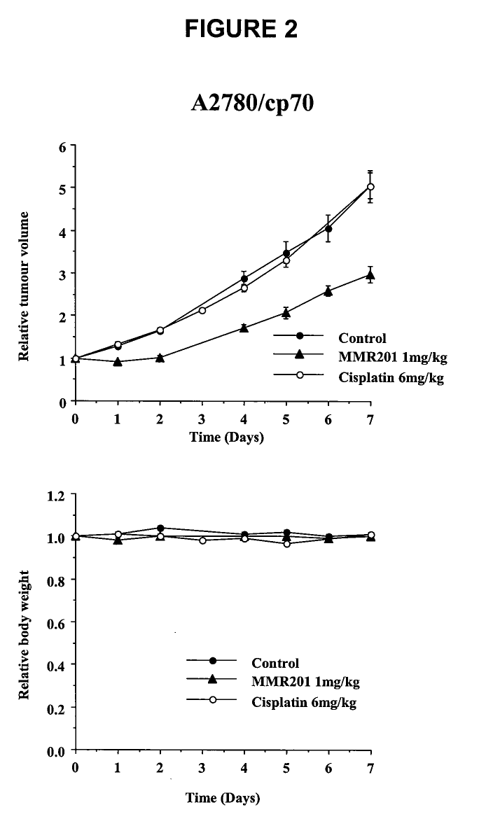

FIG. 2 shows two graphs, one of relative tumour volume versus time (days), and one of relative body weight versus time (days), for A2780/cp70 human ovarian, cisplatin resistant, cell line grown as xenograft in nude mice. Mice were treated with a single bolus injection on day 0 with (a) control, (b) MMR201 (1 mg/kg) i.v., and (c) cisplatin (6 mg/kg), i.p. Error bars represent standard error of the mean.

These data show that MMR201 can inhibit growth of MLH1 deficient, cisplatin resistant A2780/cp70 colon cells at tolerated doses in mice.

Cisplatin at maximum tolerated dose does not inhibit growth of these xenograft models.

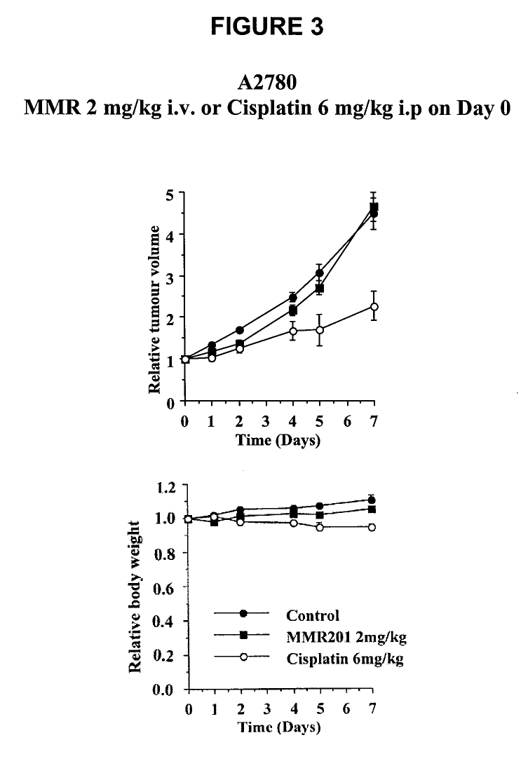

FIG. 3 shows two graphs, one of relative tumour volume versus time (days), and one of relative body weight versus time (days), for A2780 human ovarian tumour cell line grown as xenograft in nude mice. Mice were treated with a single bolus injection on day 0 with (a) control, (b) MMR201 (2 mg/kg) i.v., and (c) cisplatin (6 mg/kg), i.p. Error bars represent standard error of the mean.

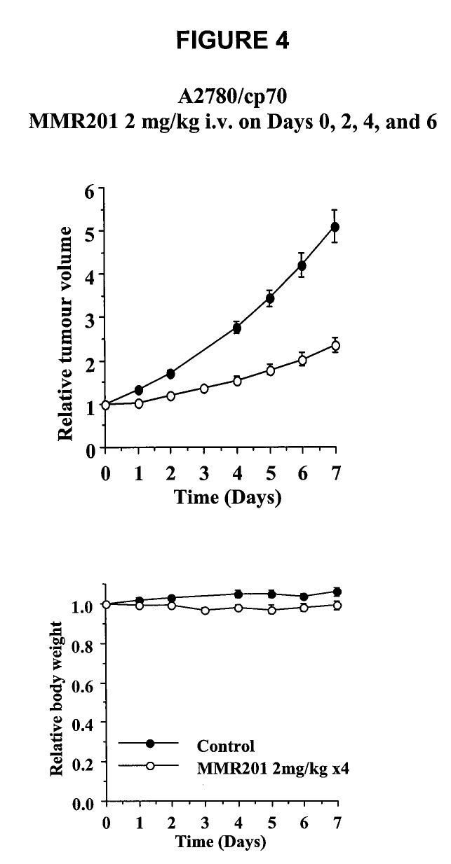

FIG. 4 shows two graphs, one of relative tumour volume versus time (days), and one of relative body weight versus time (days), for A2780/cp70 human ovarian, cisplatin resistant, cell line grown as xenograft in nude mice. Mice were treated with a bolus injection of MMR201 (2 mg/kg) i.v. on each of days 0, 2, 4, and 6. Error bars represent standard error of the mean.

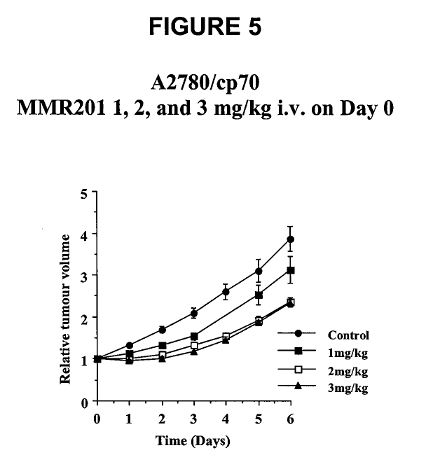

FIG. 5 shows one graphs of relative tumour volume versus time (days) for A2780/cp70 human ovarian, cisplatin resistant, cell line grown as xenograft in nude mice. Mice were treated with a single bolus injection of MMR201 (1 mg/kg, 2 mg/kg or 3 mg/kg) i.v. on day 0. Error bars represent standard error of the mean.

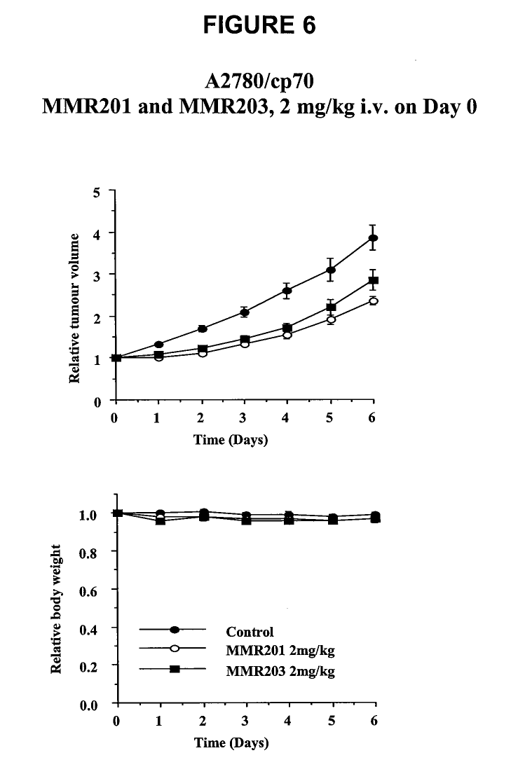

FIG. 6 shows two graphs, one of relative tumour volume versus time (days), and one of relative body weight versus time (days), for A2780/cp70 human ovarian, cisplatin resistant, cell line grown as xenograft in nude mice. Mice were treated with a single bolus injection of MMR201 (2 mg/kg) i.v. or MMR203 (2 mg/kg) i.v. on day 0. Error bars represent standard error of the mean.

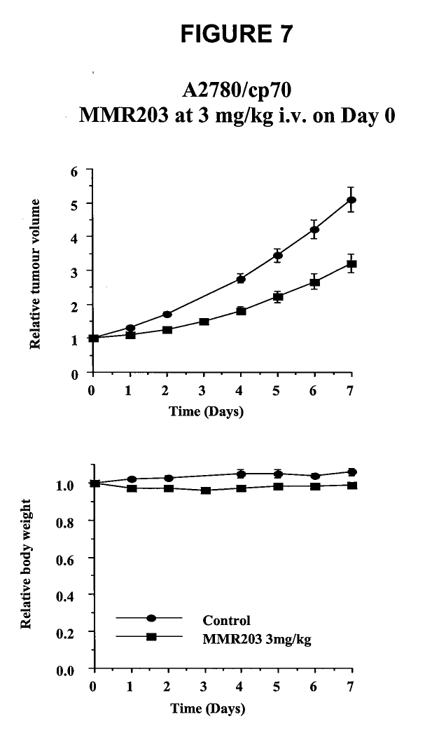

FIG. 7 shows two graphs, one of relative tumour volume versus time (days), and one of relative body weight versus time (days), for A2780/cp70 human ovarian, cisplatin resistant, cell line grown as xenograft in nude mice. Mice were treated with a single bolus injection of MMR203 (3 mg/kg) i.v. on day 0. Error bars represent standard error of the mean.

In additional studies, it was shown that MMR201 induces apoptosis but does not induce expression of p53. Apoptosis as determined by PARP cleavage was apparent after incubation with MMR201 for 24 hours and MLH1 negative cells showed increased apoptosis at lower drug concentrations. MMR201 does not induce p53 at doses that were shown to induce apoptosis.

The foregoing has described the principles, preferred embodiments, and modes of operation of the present invention. However, the invention should not be construed as limited to the particular embodiments discussed. Instead, the above-described embodiments should be regarded as illustrative rather than restrictive, and it should be appreciated that variations may be made in those embodiments by workers skilled in the art without departing from the scope of the present invention.

### REFERENCES

A number of patents and publications are cited herein in order to more fully describe and disclose the invention and the state of the art to which the invention pertains. Full citations for these references are provided herein. Each of these references is incorporated herein by reference in its entirety into the present disclosure.
Abramenko, P. I. and Priklonskikh, G. I., 1984, “Thienopyrido-, furopyrido- and thienothiazoloquino-2-monomethinecyanines and their properties,” Zhurnal Vsesoyuznogo Khimicheskogo Obshchestva im. D. I. Mendeleeva, Vol. 29, No. 1, pp. 110-111.
Abramenko, P. I. and Zhiryakov, V. G., 1977, “Synthesis of methyl-substituted thiazole bases with a condensed furan, thiophene, or selenophene ring”, Chemistry of Heterocyclic Compounds, Vol. 13, No. 11, pp. 1194-1197.
Aggarwal, R., et al., 2004, “A facile Synthesis of Thiazole-2(3H)-thiones Through [Hydroxy(tosyloxy)iodo]benzene”, Synth. Comm., Vol. 34, p. 2659.
Anthoney, D. A., et al., 1996, “Microsatellite instability, apoptosis and loss of p53 function in drug resistant tumour cells,” Cancer Research, Vol. 56, pp. 1374-1381.
Beachy, P. A., et al., 2005, “Hedgehog Pathway Antagonists,” published international (PCT) patent application publication number WO 2005/033288 A1, published 14 Apr. 2005.
Bogolyubskaya, L. T., Al'perovich, M. A., 1964, Zhurnal Obshchei Khimii, pp. 3119-3120.
Bogolyubskaya, L. T. et al., 1964, “The action of thioacetamide on 2-bromo-3-coumaranone”, J. Gen. Chem. (USSR) (Engl. Transl.), Vol. 34, pp. 3159-3160.
Boyer, J. C., et al., 1998, “Mutation rate of a microsatellite sequence in normal human fibroblasts,” Cancer Research, Vol. 58, pp. 3946-3949.
Brandsma, L. et al., 1985, “An Efficient Synthesis of 1,3-Thiazole”, Synthesis, p. 948.
Brown, R., 1999, Apoptosis and Cancer Chemotherapy (J. A. Hickman and C. Dive, Eds.) (Humana Press, Totowa, N.J.), p. 69.
Buchman, E. R. et al., 1941, “Mercaptothiazoles: Oxidation and Alkylation Studies” J. Org. Chem., Vol. 6, p. 764.
Dent, S. G., et al., 1951, “Process for Preparing Polymethine Dyes,” U.S. Pat. No. 2,537,880 published 9 Jan. 1951.
Duckett, D. R., et al., 1999, “hMutSalpha and MutLalpha dependent phosphorylation of p53 in response to DNA methylator damage,” Proc. Natl. Acad. Sci. USA, Vol. 96, pp. 12384-12388.
Durant, S. T., Morris, M. M., Illand, M., McKay, H. J., McCormick, C., Hirst, G. L., Borts, R. H., Brown, R., 1999, “Dependence on RAD52 and RAD1 for anticancer drug resistance mediated by inactivation of mismatch repair genes,” Current Biology, Vol. 9, pp. 51-54.
Eshleman, J. R., et al., 1995, “Microsatellite instability in inherited and sporadic neoplasms,” Current. Opin. Oncol., Vol. 7, pp. 83-89.
Fink, D., et al., 1998, “The role of DNA mismatch repair in drug resistance,” Clinical Cancer Research, Vol. 4, pp. 1-6.
Frey, L. F. et al., 2003, “Practical synthesis of a highly functionalized thiazole,” Tetrahedron, Vol. 59, pp. 6363-6373.
Frey, L. F., et al., 2003, “Practical synthesis of a highly functionalised thiazole ketone”, Tetrahedron, Vol. 59, p. 6363.
Fujikawa, K., et al., 1956, “A study on the action of chemotherapeutic agents on the Candida”, Igaku Kenkvu, Vol. 26, pp. 2106-2147.
Giordano, T., et al., 2003, “Inhibitors of R^{NA}SE P proteins as antibacterial compounds”, published international (PCT) patent application publication number WO 03/024407 A2, published 27 Mar. 2003.
Gong, J., et al., 1999, “The tyrosine kinase c-Abl regulates p73 in apoptotic response to cisplatin-induced DNA damage,” Nature, Vol. 399, pp. 806-809.
Hamer, F. M., 1928, “The ψ-cyanine condensation,” J. Chem. Soc., pp. 206-215.
Hamer, F. M., 1964, Cyanine Dyes and Related Compounds, Interscience Publishers, John Wiley and Sons: New York, London.
Hojo, M. et al., 1988, “Isolation and reaction of new aromatic dications. 4-Thioniapyridinium dications: 2-aryl-(or 2-alkyl-)-4-methyl-3-oxo-2H-1,4-thiazin-2-ylium tetrafluoroborates,” J. Org. Chem., Vol. 53, pp. 2209-2213.
Ihara, M., et al., 2006, “Anti-Leishmania Agent,” published European patent application publication number EP 1 623 981 A1, published 8 Feb. 2006.
Inglis, S. R., et al., 2004, “Identification and Specificity Studies of Small-Molecule Ligands for SH3 Protein Domains”, J. Med. Chem., Vol. 47, p. 5405.
Joule, J., Mills, K., Smith, G. F., 1995, Heterocyclic Chemistry, 3^{rd} Edition, Stanley Thorn Publishers.
Kane, M. F., et al., 1997, “Methylation of the hMLH1 promoter correlates with lack of expression of hMLH1 in sporadic colon tumors and mismatch repair-defective tumor cell lines,” Cancer Research, Vol. 57, pp. 808-811.
Kawakami, M., 1997, “Synthesis and Evaluation of Novel Rhodacyanine Dyes That Exhibit Antitumor Activity,” J. Med. Chem., Vol. 40, pp. 3151-3160.
Kendall, J. D., et al., 1949, “The reactivity of alkylthio-group in nitrogen ring compounds. Part 1. A general method for the preparation of symmetrical and unsymmetrical thiacyanines,” J. Chem. Soc., pp. 1503-1509.
Lee, S.-J., et al., 2006, “Benzothiazolium compounds,” published US patent application publication number US 2006/0035926 A1, published 16 Feb. 2006.
Li, P., Xu, J., 2000, “1-Ethyl 2-Halopyridinium Salts, Highly Efficient Coupling Reagents for Hindered Peptide Synthesis both in Solution and the Solid-Phase”, Tetrahedron, Vol. 56, No. 41, pp. 8119-8131.
Lynch, H. T., 1993, “Genetics, Natural history, tumor spectrum and pathology of hereditary nonpolyposis colorectal cancer: An updated review,” Gasteroenterology, Vol. 104, pp. 1535-1549.
Mackay, H. J., et al., 2000, “Reduced MLH1 expression in breast tumours after primary chemotherapy predicts disease free survival,” J. Clinical Oncology, Vol. 18, pp. 87-93.
Miroshnichenko, Z. I. et al., 1964a, “Polymethine Dyes from Isomeric 2-Methylthionaphthenonthiazoles. I. Cyanine Dyes, Derivatives of 2-Methyl 4,5-(2′,3′-Thionaphtheno)thiazole,” J. Gen. Chem. (USSR) (Engl. Transl.), Vol. 34, pp. 239-244.
Miroshnichenko, Z. I., et al., 1964b, “Polymethine Dyes from Isomeric 2-Methylthionaphthenothiazoles. II. Cyanine Dyes, Derivatives of 2-methyl-4,5-(3′,2′-thionaphtheno)thiazoles,” J. Gen. Chem. (USSR) (Engl. Transl.), Vol. 34, pp. 245-248.
Mosman, T., 1983, “Rapid colorimetric assay for cellular growth and survival: application to proliferation and cytotoxicity assays,” Journal of Immunological Methods, Vol. 65, pp. 55-63.
Nieland, T. J. F., et al., 2004, “Compounds for Modulation of Cholesterol Transport,” published international (PCT) patent application publication number WO 2004/032716 A2, published 22 Apr. 2004.
Nomura, H. T., et al., 1979, “Tumorhemmendes Mittel,” German patent publication number DE 29 19 447, published 22 Nov. 1979.
Nomura, Hiroaki, et al., 1979, “Compositions Containing Cyanine Compounds,” published United Kingdom patent application number 2 020 975 A, published 28 Nov. 1979.
Plumb, J. A., Milroy, R., Kaye, S. B., 1989, “Effects of the pH dependence of 3-(4,5-dimethylthiazol-2-yl)-2,5-diphenyltetrazolium bromide-formazan absorption on chemosensitivity determined by a novel tetrazolium-based assay,” Cancer Research, Vol. 49, pp. 4435-4440.
Shumelyak, G. P. et al., 1964 “Some Polymethine Dyes with Radical of 4,5-(2′-Methylthiazolo-5′,4′)thaizole,” J. Gen. Chem. USSR (Engl. Transl.), Vol. 34, pp. 249-251.
Strathdee, G., et al., 1999, “A role for methylation of the hMLH1 promoter in loss of hMLH1 expression and drug resistance in ovarian cancer,” Oncogene, Vol. 18, pp. 1335-1341.
Tseng, H.-Y., et al., 2005, “Benzothiazolium compounds: novel classes of inhibitors that suppress the nitric oxide production in RAW264.7 cells stimulated by LPS/IFNγ,” Bioorganic & Medicinal Chemistry Letters, Vol. 15, pp. 2027-2032.

1. A compound selected from compounds of the following formula: 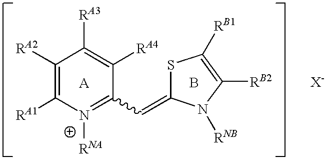
wherein:
R^{NA} and X⁻ may be present, or R^{NA} and X⁻ may be absent;
if R^{NA} and X⁻ are present, then:
the nitrogen atom of the A-ring bears a positive charge;
if R^{NA} and X⁻ are absent, then:
the nitrogen atom of the A-ring is electrically neutral;
and wherein:
(1-a) each of R^{A1}, R^{A2}, R^{A3}, and R^{A4} is independently an A-ring monovalent monodentate substituent; or:
(1-b) each of R^{A3} and R^{A4} is independently an A-ring monovalent monodentate substituent, and R^{A1} and R^{A2}, together with the carbon ring atoms of the A-ring to which they are attached, form a D-ring that is fused to the A-ring and which is:
(1-b)(i) a 6-membered carboaromatic ring, or
(1-b)(ii) a 5-membered heteroaromatic ring having exactly 1 heteroatom ring atom, which heteroatom is selected from N, O, and S, or
(1-b)(iii) a 5-membered heteroaromatic ring having exactly 2 heteroatom ring atoms, which heteroatoms are selected from N, O, and S;
and wherein the D-ring is unsubstituted, or is substituted with one or more D-ring substituents, provided that D-ring substituents do not form a further ring; or:
(1-c) each of R^{A1} and R^{A4} is independently an A-ring monovalent monodentate substituent, and R^{A2} and R^{A3}, together with the carbon ring atoms of the A-ring to which they are attached, form an E-ring that is fused to the A-ring and which is:
(1-c)(i) a 6-membered carboaromatic ring, or
(1-c)(ii) a 5-membered heteroaromatic ring having exactly 1 heteroatom ring atom, which heteroatom is selected from N, O, and S, or
(1-c)(iii) a 5-membered heteroaromatic ring having exactly 2 heteroatom ring atoms, which heteroatoms are selected from N, O, and S;
and wherein the E-ring is unsubstituted, or is substituted with one or more E-ring substituents, provided that E-ring substituents do not form a further ring; or:
(1-d) each of R^{A1} and Re is independently an A-ring monovalent monodentate substituent, and R^{A3} and R^{A4}, together with the carbon ring atoms of the A-ring to which they are attached, form a C-ring that is fused to the A-ring and which is:
(1-d)(i) a 6-membered carboaromatic ring, or
(1-d)(ii) a 5-membered heteroaromatic ring having exactly 1 heteroatom ring atom, which heteroatom is selected from N, O, and S, or
(1-d)(iii) a 5-membered heteroaromatic ring having exactly 2 heteroatom ring atoms, which heteroatoms are selected from N, O, and S;
and wherein the C-ring is unsubstituted, or is substituted with one or more C-ring substituents, provided that C-ring substituents do not form a further ring;
and wherein:
(2-a) each of R^{B1} and R^{B2} is independently a B-ring monovalent monodentate substituent; or:
(2-b) R^{B1} and R^{B2}, together with the carbon ring atoms of the B-ring to which they are attached, form an F-ring that is fused to the B-ring and which is:
(i) a 5-membered heteroaromatic ring having exactly 1 heteroatom ring atom, which heteroatom is selected from N, O, and S, or
(ii) a 5-membered heteroaromatic ring having exactly 2 heteroatom ring atoms, which heteroatoms are selected from N, O, and S;
and wherein the F-ring is unsubstituted, or is substituted with one or more F-ring substituents, provided that F-ring substituents do t not form a further ring; or:
(2-c) R^{B1} and R^{B2}, together with the carbon ring atoms of the B-ring to which they are attached, form an F-ring that is fused to the B-ring and which is a 5-membered heteroaromatic ring having exactly 1 heteroatom ring atom, which heteroatom is selected from N, O, and S,
wherein the F-ring is substituted with two adjacent F-ring substituents which, together with the carbon ring atoms of the F-ring to which they are attached, form a G-ring that is fused to the F-ring and which is:
(2-c)(i) a 6-membered carboaromatic ring, or
(2-c)(ii) a 5-membered heteroaromatic ring having exactly 1 heteroatom ring atom, which heteroatom is selected from N, O, and S, or
(2-c)(iii) a 5-membered heteroaromatic ring having exactly 2 heteroatom ring atoms, which heteroatoms are selected from N, O, and S;
and wherein the G-ring is unsubstituted, or is substituted with one or more G-ring substituents, provided that G-ring substituents do not form a further ring;
and wherein:
R^{NA}, if present, is independently an A-ring nitrogen substituent;
R^{NB} is independently a B-ring nitrogen substituent;
the linkage joining the A-ring and the B-ring is cis or trans;
and pharmaceutically acceptable salts, solvates, amides, esters, ethers, N-oxides, chemically protected forms, and prodrugs thereof;
with the proviso that the compound is not one of the compounds denoted (P-01) to (P-15) and (Q-01) to (Q-09);
and wherein:
each D-ring substituent, if present, is independently a monovalent monodentate substituent or oxo;
each E-ring substituent, if present, is independently a monovalent monodentate substituent or oxo;
each C-ring substituent, if present, is independently a monovalent monodentate substituent or oxo;
each F-ring substituent, if present, is independently a monovalent monodentate substituent or oxo;
each G-ring substituent, if present, is independently a monovalent monodentate substituent or oxo;
and wherein each monovalent monodentate substituent is independently selected from:
(1) —H;
(2) —F; —Cl; —Br; —I;
(3) —OH; —OR;
(4) —SH; —SR;
(5) —NO₂;
(6) —C(═O)R;
(7) —C(═O)OH; —C(═O)OR;
(8) —C(═O)NH₂; —C(═O)NHR; —C(═O)NR₂; —C(═O)NR^{N1}R^{N2};
(9) —NH₂; —NHR; —NR₂; —NR^{N1}R^{N2};
(10) —NHC(═O)H; —NRC(═O)H; —NHC(═O)R; —NRC(═O)R;
(11) —R;
(12) —R^{Alk}—OH; —R^{Alk}—OR
(13) —R^{Alk}—NH₂; —R^{Alk}—NHR; —R^{Alk}—NR₂; —R^{Alk} NR^{N1}R^{N2};
(14) —R^{Alk}—C(═O)OH; —R^{Alk}—C(═O)OR;
(15) —R^{Alk}C(═O)NH₂; —R^{Alk}—C(═O)NHR; —R^{Alk}—C(═O)NR₂; —R^{Alk}—C(═O)NR^{N1}R^{N2};
(16) -J-R^{Alk}—OH; -J-R^{Alk}—OR
(17) -J-R^{Alk}—NH₂; -J-R^{Alk}—NHR; -J-R^{Alk}—NR₂; -J-R^{Alk}—NR^{N1}NR^{N2};
(18) -J-R^{Alk}—C(═O)OH; -J-R^{Alk}—C(═O)OR;
(19) -J-R^{Alk}—C(═O)NH₂; -J-R^{Alk}—C(═O)NHR;
(20) -J-R^{Alk}—C(═O)NR; -J-R^{Alk}—C(═O)NR^{N1}R^{N2};
wherein each J is independently selected from —O—, —NH—, or —NR—;
wherein each R is independently selected from:
(a) unsubstituted aliphatic C₁₋₆alkyl; substituted aliphatic C₁₋₆alkyl;
(b) unsubstituted aliphatic C₂₋₆alkenyl; substituted aliphatic C₂₋₆alkenyl;
(c) unsubstituted C₃₋₆cycloalkyl; substituted C₃₋₆cycloalkyl;
(d) unsubstituted C₆₋₁₀carboaryl; substituted C₆₋₁₀carboaryl;
(e) unsubstituted C₅₋₁₀heteroaryl; substituted C₅₋₁₀heteroaryl;
(f) unsubstituted C₆₋₁₀carboaryl-aliphatic C₁₋₄alkyl;
(g) substituted C₁₋₁₀carboaryl-aliphatic C₁₋₄alkyl;
(h) unsubstituted C₅₋₁₀heteroaryl-aliphatic C₁₋₄alkyl;
(i) substituted C₅₋₁₀heteroaryl-aliphatic C₁₋₄alkyl;
wherein each R^{Alk} is independently selected from:
(I) unsubstituted aliphatic C1-6alkylene; substituted aliphatic C1-6alkylene;
(II) unsubstituted aliphatic C2-6alkenylene; substituted aliphatic C2-6alkenylene;
(III) unsubstituted C3-6cycloalkylene; substituted C3-6cycloalkylene;
wherein in each group —NR^{N1}R^{N2}, independently, R^{N1} and R^{N2} taken together with the nitrogen atom to which they are attached form a ring having from 3 to 7 ring atoms;
and wherein R^{NA}, if present, is independently selected from:
(1) —H;
(11) —R;
(12) —R^{Alk}—OH; —R^{Alk}—OR
(13) —R^{Alk}—NH₂; —R^{Alk}—NHR; —R^{Alk}—NR₂; —R^{Alk}—NR^{N1}R^{N2};
(14) —R^{Alk}—C(═O)OH; —R^{Alk}—C(═O)OR;
(15) —R^{Alk}—C(═O)NH₂; R^{Alk}—C(═O)NHR; —R^{Alk}—C(═O)NR₂; —R^{Alk}—C(═O)NR^{N1}R^{N2};
wherein each R is independently selected from:
(a) unsubstituted aliphatic C₁₋₆alkyl; substituted aliphatic C₁₋₆alkyl;
(b) unsubstituted aliphatic C₂₋₆alkenyl; substituted aliphatic C₂₋₆alkenyl;
(c) unsubstituted C₃₋₆cycloalkyl; substituted C₃₋₆cycloalkyl;
(d) unsubstituted C₆₋₁₀carboaryl; substituted C₆₋₁₀carboaryl;
(e) unsubstituted C₅₋₁₀heteroaryl; substituted C₅₋₁₀heteroaryl;
(f) unsubstituted C₆₋₁₀carboaryl-aliphatic C₁₋₄alkyl;
(g) substituted C₆₋₁₀carboaryl-aliphatic C₁₋₄alkyl;
(h) unsubstituted C₆₋₁₀heteroaryl-aliphatic C₁₋₄alkyl;
(i) substituted C₅₋₁₀heteroaryl-aliphatic C₁₋₄alkyl;
wherein each R^{Alk} is independently selected from:
(I) unsubstituted aliphatic C₁₋₆alkylene; substituted aliphatic C₁₋₆alkylene;
(II) unsubstituted aliphatic C₂₋₆alkenylene; substituted aliphatic C₂₋₆alkenylene;
(III) unsubstituted C₃₋₆cycloalkylene; substituted C₃₋₆cycloalkylene;
wherein in each group —NR^{N1}R^{N2}, independently, R^{N1} and R^{N2} taken together with the nitrogen atom to which they are attached form a ring having from 3 to 7 ring atoms;
and wherein R^{NB} if present, is independently selected from:
(1) —H;
(11) —R;
(12) —R^{Alk}—OH; —R^{Alk}—OR
(13) —R^{Alk}—NH₂; —R^{Alk}—NHR; —R^{Alk}—NR₂; —R^{Alk}—NR^{N1}R^{N2};
(14) R^{Alk}—C(═O)OH; —R^{Alk}—C(═O)OR;
(15) —R^{Alk}—C(═O)NH₂; —R^{Alk}—C(═O)NHR; —R^{Alk}—C(═O)NR₂; —R^{Alk}—C(═O)NR^{N1}R^{N2};
wherein each R is independently selected from:
(a) unsubstituted aliphatic C₁₋₆alkyl; substituted aliphatic C₁₋₆alkyl;
(b) unsubstituted aliphatic C₂₋₆alkenyl; substituted aliphatic C₂₋₆alkenyl;
(c) unsubstituted C₃₋₆cycloalkyl; substituted C₃₋₆cycloalkyl;
(d) unsubstituted C₆₋₁₀carboaryl; substituted C₆₋₁₀carboaryl;
(e) unsubstituted C₅₋₁₀heteroaryl; substituted C₅₋₁₀heteroaryl;
(f) unsubstituted C₆₋₁₀carboaryl-aliphatic C₁₋₄alkyl;
(g) substituted C₆₋₁₀carboaryl-aliphatic C₁₋₄alkyl;
(h) unsubstituted C₅₋₁₀heteroaryl-aliphatic C₁₋₄alkyl;
wherein each R^{Alk} is independently selected from:
(I) unsubstituted aliphatic C₁₋₆alkylene; substituted aliphatic C₁₋₆alkylene;
(II) unsubstituted aliphatic C₂₋₆alkenylene; substituted aliphatic C₂₋₆alkenylene;
(III) unsubstituted C₃₋₆cycloalkylene; substituted C₃₋₆cycloalkylene;
wherein in each group —NR^{N1}R^{N2}, independently, R^{N1} and R^{N2} taken together with the nitrogen atom to which they are attached form a ring having from 3 to 7 ring atoms.

2. A compound according to claim 1, wherein the linkage joining the A-ring and the B-ring is cis.

3. A compound according to claim 1, wherein the linkage joining the A-ring and the B-ring is trans.

4. A compound according to claim 1, wherein R^{NA} and X⁻ are present, the nitrogen atom of the A-ring bears a positive charge.

5. (canceled)

6. A compound according to claim 1, wherein:
(1-a) each of R^{A1}, R^{A2}, R^{A3}, and R^{A4} is independently an A-ring monovalent monodentate substituent.

7-9. (canceled)

10. A compound according to claim 1, wherein the D-ring is as depicted in the following formula, wherein w is 0, 1, 2, 3, or 4, and each R^{D} is independently a D-ring substituent:

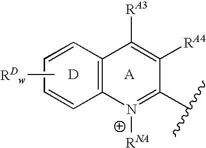

11-68. (canceled)

69. A compound according to claim 1, wherein the F-ring and G-ring are as depicted in one of the following formulae, wherein w is 0, 1, 2, 3, or 4 and each R^{G} is independently a G-ring substituent:

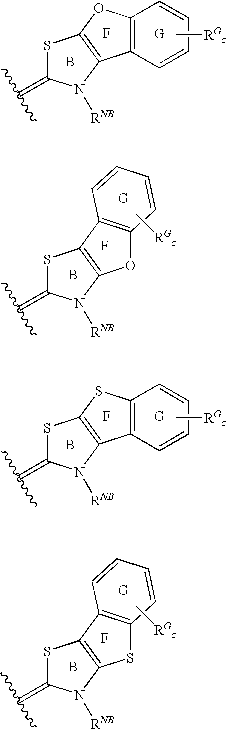

70-89. (canceled)

90. A compound according to claim 1, wherein each monovalent monodentate substituent is independently selected from:
—H;
—F; —Cl; —Br; —I;
—OH;
—OMe, —OEt, —O(nPr), —O(iPr);
—SH;
—SMe, —SEt, —S(nPr), —S(iPr);
—NO₂;
—C(═O)Me, —C(═O)Et, —C(═O)(nPr), —C(═O)(iPr);
—C(═O)OH;
—C(═O)OMe, —C(═O)OEt, —C(═O)O(nPr), —C(═O)O(iPr);
—C(═O)NH₂;
—C(═O)NHMe, —C(═O)NHEt, —C(═O)NH(nPr), —C(═O)NH(iPr);
—C(═O)NMe₂, —C(═O)NEt₂, —C(═O)N(nPr)₂, —C(═O)N(iPr)₂;
—C(═O)-pyrrolidino, —C(═O)-piperidino, —C(═O)-piperazino, —C(═O)-morpholino;
—NH₂;
—NHMe, —NHEt, —NH(nPr), —NH(iPr); —NMe₂, —NEt₂, —N(nPr)₂, —N(iPr)₂;
pyrrolidino, piperidino, piperazino, morpholino;
—NHC(═O)H;
—NMeC(═O)H, —NEtC(═O)H, —N(nPr)C(═O)H, —N(iPr)C(═O)H;
—NHC(═O)Me, —NHC(═O)Et, —NHC(═O)(nPr), —NHC(═O)(iPr);
—NMeC(═O)Me, —NEtC(═O)Me, —N(nPr)C(═O)Me, —N(iPr)C(═O)Me;
-Me, -Et, -nPr, -iPr;
phenyl, pyrrolyl, thienyl, furyl, imidazolyl, oxazolyl, isoxazolyl, thiazolyl, isothiazolyl, pyrazolyl, pyridyl, pyrazinyl, pyrimidinyl, pyridazinyl, benzyl, phenylethyl, pyridyl-methyl, isothiazolyl-methyl, each optionally substituted with one or more of —F, —Cl, —Br, —I, —OH, —OMe, —OEt, —O(nPr), —O(iPr), —NH₂, —NHMe, —NHEt, —NH(nPr), —NH(iPr), —NMe₂, —NEt₂, —N(nPr)₂, —N(iPr)₂, pyrrolidino, piperidino, piperazino, morpholino, —COOH, —COMe, —COEt, —CO(nPr), —CO(iPr), —CONH₂, —CONHMe, —CONHEt, —CONH(nPr), —CONH(iPr), —CONMe₂, —CONEt₂, —CON(nPr)₂, —CON(iPr)₂, -Me, -Et, -nPr, -iPr;
—(CH₂)ₘ—OH;
—(CH₂)ₘ—OMe, —(CH₂)ₘ—OEt, —(CH₂)ₘ—O(nPr), —(CH₂)ₘ—O(iPr);
—(CH₂)ₘ—COOH;
—(CH₂)ₘ—COOMe, —(CH₂)ₘ—COOEt, —(CH₂)ₘ—COO(nPr), —(CH₂)ₘ—COO(iPr);
—(CH₂)ₘ—NH₂;
—(CH₂)ₘ—NHMe, —(CH₂)ₘ—NMe₂, —(CH₂)ₘ—NHEt, —(CH₂)ₘ—NEt₂, —(CH₂)ₘ—NH(nPr), —(CH₂)ₘ—N(nPr)₂, —(CH₂)ₘ—NH(iPr), —(CH₂)ₘ—N(iPr)₂, —(CH₂)ₘ-pyrrolidino, —(CH₂)ₘ-piperidino, —(CH₂)ₘ-piperazino, —(CH₂)ₘ-morpholino;
—(CH₂)ₘ—CONH Me, —(CH₂)ₘ—CONMe₂, —(CH₂)ₘ—CONHEt, —(CH₂)ₘ—CONEt₂, —(CH₂)ₘ—CONH(nPr), —(CH₂)ₘ—CON(nPr)₂, —(CH₂)ₘ—CONH(iPr), —(CH₂)ₘ—CON(iPr)₂, —(CH₂)ₘ—CO-pyrrolidino, —(CH₂)ₘ—CO-piperidino, —(CH₂)ₘ—CO-piperazino, —(CH₂)ₘ—CO-morpholino;
—O—(CH₂)ₘ—OH; —O—(CH₂)ₘ—OMe, —O—(CH₂)ₘ—OEt, —O—(CH₂)ₘ—O(nPr), —O—(CH₂)ₘ—O(iPr);
—O—(CH₂)ₘ—COOH;
—O—(CH₂)ₘ—COOMe, —O—(CH₂)ₘ—COOEt, —O—(CH₂)ₘ—COO(nPr), —O—(CH₂)ₘ—COO(iPr);
—O—(CH₂)ₘ—NH₂;
—O—(CH₂)ₘ—NHMe, —O—(CH₂)ₘ—NMe₂, —O—(CH₂)ₘ—NHEt, —O—(CH₂)ₘ—NEt₂, —O—(CH₂)ₘ—NH(nPr), —O—(CH₂)ₘ—N(nPr)₂, —O—(CH₂)ₘ—NH(iPr), —O—(CH₂)ₘ—N(iPr)₂, —O—(CH₂)ₘ-pyrrolidino, —O—(CH₂)ₘ-piperidino, —O—(CH₂)ₘ-piperazino, —O—(CH₂)ₘ-morpholino;
—O—(CH₂)ₘ—CONHMe, —O—(CH₂)ₘ—CONMe₂, —O—(CH₂)ₘ—CON H Et, —O—(CH₂)ₘ—CON Et₂, —O—(CH₂)ₘ—CONH(nPr), —O—(CH₂)ₘ—CON(nPr)₂, —O—(CH₂)ₘ—CONH(iPr), —O—(CH₂)ₘ—CON(iPr)₂, —O—(CH₂)ₘ—CO-pyrrolidino, —O—(CH₂)ₘ—CO-piperidino, —O—(CH₂)ₘ—CO-piperazino, —O—(CH₂)ₘ—CO-morpholino;
—NH—(CH₂)ₘ—OH;
—NH—(CH₂)ₘ—OMe, —NH—(CH₂)ₘ—OEt, —NH—(CH₂)ₘ—O(nPr), —NH—(CH₂)ₘ—O(iPr);
—NH—(CH₂)ₘ—COOH;
—NH—(CH₂)ₘ—COOMe, —NH—(CH₂)ₘ—COOEt, —NH—(CH₂)ₘ—COO(nPr), —NH—(CH₂)ₘ—COO(iPr);
—NH—(CH₂)ₘ—NH₂;
—NH—(CH₂)ₘ—NHMe, —NH—(CH₂)ₘ—NMe₂, —NH—(CH₂)ₘ—NHEt, —NH—(CH₂)ₘ, —NEt₂, —NH—(CH₂)ₘ—NH(nPr), —NH—(CH₂)ₘ—N(nPr)₂, —NH—(CH₂)ₘ—NH(iPr), —NH—(CH₂)ₘ—N(iPr)₂, —NH—(CH₂)ₘ-pyrrolidino, —NH—(CH₂)ₘ-piperidino, —NH—(CH₂)ₘ-piperazino, —NH—(CH₂)ₘ-morpholino;
—NH—(CH₂)ₘ—CONHMe, —NH—(CH₂)ₘ—CONMe₂, —NH—(CH₂)ₘ—CONHEt, —NH—(CH₂)ₘ—CONEt₂, —NH—(CH₂)ₘ—CONH(nPr), —NH—(CH₂)ₘ—CON(nPr)₂, —NH—(CH₂)ₘ—CONH(iPr), —NH—(CH₂)ₘ—CON(iPr)₂, —NH—(CH₂)ₘ—CO-pyrrolidino, —NH—(CH₂)ₘ—CO-piperidino, —NH—(CH₂)ₘ—CO-piperazino, —NH—(CH₂)ₘ—CO-morpholino;
wherein each m is independently 1, 2, 3, or 4.

91. A compound according to claim 1, wherein each monovalent monodentate substituent is independently selected from:
—H;
—Cl;
—OH;
—OMe;
—C(═O)NH₂;
—C(═O)NHMe, —C(═O)NHEt;
-Me, -Et, -iPr;
phenyl, pyridyl, each optionally substituted with one or more of —F, —Cl, —OH, —OMe, —CONH₂, —CONHMe, —CONHEt, -Me, -Et, and -iPr;
—(CH₂)ₘ—OH;
—(CH₂)ₘ—OMe;
—(CH₂)ₘ—NH₂;
—(CH₂)ₘ—NMe₂, —(CH₂)ₘ—NEt₂, —(CH₂)ₘ-morpholino;
—(CH₂)ₘ—CONHMe, —(CH₂)ₘ—CONHEt;
—O—(CH₂)ₘ—OH;
—O—(CH₂)ₘ—NH₂;
—O—(CH₂)ₘ—NMe₂, —O—(CH₂)ₘ—NEt₂, —O—(CH₂)ₘ-morpholino;
wherein each m is independently 1, 2, or 3.

92. (canceled)

93. A compound according to claim 1, wherein R^{NA}, if present, is independently selected from:
—H;
-Me, -Et, -nPr, -iPr;
—(CH₂)ₘ—OH;
—(CH₂)ₘ—O Me, —(CH₂)ₘ—OEt, —(CH₂)ₘ—O(nPr), —(CH₂)ₘ—O(iPr);
—(CH₂)ₘ—COOH;
—(CH₂)ₘ—COOMe, —(CH₂)ₘ—COOEt, —(CH₂)ₘ—COO(nPr), —(CH₂)ₘ—COO(iPr);
—(CH₂)ₘ—NH₂;
—(CH₂)ₘ—NHMe, —(CH₂)ₘ—NMe₂, —(CH₂)ₘ—NHEt, —(CH₂)ₘ—NEt₂, —(CH₂)ₘ—NH(nPr), —(CH₂)ₘ—N(nPr)₂, —(CH₂)ₘ—NH(iPr), —(CH₂)ₘ—N(iPr)₂, —(CH₂)ₘ-pyrrolidino, —(CH₂)ₘ-piperidino, —(CH₂)ₘ-piperazino, —(CH₂)ₘ-morpholino;
—(CH₂)ₘ—CONHMe, —(CH₂)ₘ—CONMe₂, —(CH₂)ₘ—CONHEt, —(CH₂)ₘ—CON Et₂, —(CH₂)ₘ—CONH(nPr), —(CH₂)ₘ—CON(nPr)₂, —(CH₂)ₘ—CONH(iPr), —(CH₂)ₘ—CON(iPr)₂, —(CH₂)ₘ—CO-pyrrolidino, —(CH₂)ₘ—CO-piperidino, —(CH₂)ₘ—CO-piperazino, —(CH₂)M-CO-morpholino;
wherein each m is independently 1, 2, 3, or 4.

94. A compound according to claim 1, wherein R^{NA}, if present, is independently selected from:
—H;
-Me, -Et, -iPr;
—(CH₂)ₘ—OH;
—(CH₂)ₘ—NH₂;
—(CH₂)ₘ—NMe₂, —(CH₂)ₘ—NEt₂, and —(CH₂)ₘ-morpholino;
wherein each m is independently 2 or 3.

95. A compound according to claim 1, wherein R^{NA}, if present, is independently selected from: —H, -Me, and -Et.

96. (canceled)

97. (canceled)

98. A compound according to claim 1, wherein R^{NB}, if present, is independently selected from:
—H;
-Me, -Et, -nPr, -iPr;
—(CH₂)ₘ—OH;
—(CH₂)ₘ—OMe, —(CH₂)ₘ, —OEt, —(CH₂)ₘ—O(nPr), —(CH₂)ₘ—O(iPr);
—(CH₂)ₘ—COOH;
—(CH₂)ₘ—COOMe, —(CH₂)ₘ—COOEt, —(CH₂)ₘ—COO(nPr), —(CH₂)ₘ—COO(iPr);
—(CH₂)ₘ—NH₂;
—(CH₂)ₘ—NHMe, —(CH₂)ₘ—NMe₂, —(CH₂)ₘ—NHEt, —(CH₂)ₘ—NEt₂, —(CH₂)ₘ—NH(nPr), —(CH₂)ₘ—N(nPr)₂, —(CH₂)ₘ—NH(iPr), —(CH₂)ₘ—N(iPr)₂, —(CH₂)ₘ-pyrrolidino, —(CH₂)ₘ-piperidino, —(CH₂)ₘ-piperazino, —(CH₂)ₘ-morpholino;
—(CH₂)ₘ—CONHMe, —(CH₂)ₘ—CONMe₂, —(CH₂)ₘ—CONHEt, —(CH₂)ₘ—CONEt₂, —(CH₂)ₘ—CONH(nPr), —(CH₂)ₘ—CON(nPr)₂, —(CH₂)ₘ—CONH(iPr), —(CH₂)ₘ—CON(iPr)₂, —(CH₂)ₘ—CO-pyrrolidino, —(CH₂)ₘ—CO-piperidino, —(CH₂)ₘ—CO-piperazino, —(CH₂)ₘ—CO-morpholino;
wherein each m is independently 1, 2, 3, or 4.

99. A compound according to claim 1, wherein R^{NB}, if present, is independently selected from:
—H;
-Me, -Et, -iPr;
—(CH₂)ₘ—OH;
—(CH₂)ₘ—NH₂;
—(CH₂)ₘ—NMe₂, —(CH₂)ₘ—NEt₂, and —(CH₂)ₘ-morpholino;
wherein each m is independently 2 or 3.

100. A compound according to claim 1, wherein R^{NB}, if present, is independently selected from: —H, -Me, and -Et.

101. (canceled)

102. (canceled)

103. A composition comprising a compound according to claim 1, and a pharmaceutically acceptable carrier or diluent.

104. A method of inhibiting cell proliferation, in vitro or in vivo, comprising contacting the cell with an effective amount of a compound of the following formula: 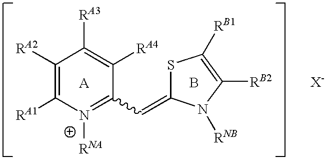
wherein:
R^{NA} and X⁻ may be present, or R^{NA} and X⁻ may be absent;
if R^{NA} and X⁻ are present, then:
the nitrogen atom of the A-ring bears a positive charge;
if R^{NA} and X⁻ are absent, then:
the nitrogen atom of the A-ring is electrically neutral;
and wherein:
(1-a) each of R^{A1}, R^{A2}, R^{A3}, and R^{A4} is independently an A-ring monovalent monodentate substituent; or:
(1-b) each of R^{A3} and R^{A4} is independently an A-ring monovalent monodentate substituent, and R^{A1} and R^{A2}, together with the carbon ring atoms of the A-ring to which they are attached, form a D-ring that is fused to the A-ring and which is:
(1-b)(i) a 6-membered carboaromatic ring, or
(1-b)(ii) a 5-membered heteroaromatic ring having exactly 1 heteroatom ring atom, which heteroatom is selected from N, O, and S, or
(1-b)(iii) a 5-membered heteroaromatic ring having exactly 2 heteroatom ring atoms, which heteroatoms are selected from N, O, and S;
and wherein the D-ring is unsubstituted, or is substituted with one or more D-ring substituents, provided that D-ring substituents do not form a further ring; or:
(1-c) each of R^{A1} and R^{A4} is independently an A-ring monovalent monodentate substituent, and R^{A2} and R^{A3}, together with the carbon ring atoms of the A-ring to which they are attached, form an E-ring that is fused to the A-ring and which is:
(1-c)(i) a 6-membered carboaromatic ring, or
(1-c)(ii) a 5-membered heteroaromatic ring having exactly 1 heteroatom ring atom, which heteroatom is selected from N, O, and S, or
(1-c)(iii) a 5-membered heteroaromatic ring having exactly 2 heteroatom ring atoms, which heteroatoms are selected from N, O, and S;
and wherein the E-ring is unsubstituted, or is substituted with one or more E-ring substituents, provided that E-ring substituents do not form a further ring; or:
(1-d) each of R^{A1} and R^{A2} is independently an A-ring monovalent monodentate substituent, and R^{A3} and R^{A4}, together with the carbon ring atoms of the A-ring to which they are attached, form a C-ring that is fused to the A-ring and which is:
(1-d)(i) a 6-membered carboaromatic ring, or
(1-d)(ii) a 5-membered heteroaromatic ring having exactly 1 heteroatom ring atom, which heteroatom is selected from N, O, and S, or
(1-d)(iii) a 5-membered heteroaromatic ring having exactly 2 heteroatom ring atoms, which heteroatoms are selected from N, O, and S;
and wherein the C-ring is unsubstituted, or is substituted with one or more C-ring substituents, provided that C-ring substituents do not form a further ring;
and wherein:
(2-a) each of R^{B1} and R^{B2} is independently a B-ring monovalent monodentate substituent; or:
(2-b) R^{B1} and R^{B2}, together with the carbon ring atoms of the B-ring to which they are attached, form an F-ring that is fused to the B-ring and which is:
(i) a 5-membered heteroaromatic ring having exactly 1 heteroatom ring atom, which heteroatom is selected from N, O, and S, or
(ii) a 5-membered heteroaromatic ring having exactly 2 heteroatom ring atoms, which heteroatoms are selected from N, O, and S;
and wherein the F-ring is unsubstituted, or is substituted with one or more F-ring substituents, provided that F-ring substituents do not form a further ring; or:
(2-c) R^{B1} and R^{B2}, together with the carbon ring atoms of the B-ring to which they are attached, form an F-ring that is fused to the B-ring and which is a 5-membered heteroaromatic ring having exactly 1 heteroatom ring atom, which heteroatom is selected from N, O, and S.
wherein the F-ring is substituted with two adjacent F-ring substituents which, together with the carbon ring atoms of the F-ring to which they are attached, form a G-ring that is fused to the F-ring and which is:
(2-c)(i) a 6-membered carboaromatic ring, or
(2-c)(ii) a 5-membered heteroaromatic ring having exactly 1 heteroatom ring atom, which heteroatom is selected from N, O, and S, or
(2-c)(iii) a 5-membered heteroaromatic ring having exactly 2 heteroatom ring atoms, which heteroatoms are selected from N, O, and S;
and wherein the G-ring is unsubstituted, or is substituted with one or more G-ring substituents, provided that G-ring substituents do not form a further ring;
and wherein:
R^{NA}, if present, is independently an A-ring nitrogen substituent;
R^{NB} is independently a B-ring nitrogen substituent;
the linkage joining the A-ring and the B-ring is cis or trans;
and pharmaceutically acceptable salts, solvates, amides, esters, ethers, N-oxides, chemically protected forms, and prodrugs thereof;
and wherein:
each D-ring substituent, if present, is independently a monovalent monodentate substituent or oxo;
each E-ring substituent, if present, is independently a monovalent monodentate substituent or oxo;
each C-ring substituent, if present, is independently a monovalent monodentate substituent or oxo;
each F-ring substituent, if present, is independently a monovalent monodentate substituent or oxo;
each G-ring substituent, if present, is independently a monovalent monodentate substituent or oxo;
and wherein each monovalent monodentate substituent is independently selected from:
(1) —H;
(2) —F; —Cl; —Br; —I;
(3) —OH; —OR;
(4) —SH; —SR;
(5) —NO₂;
(6) —C(═O)R;
(7) —C(═O)OH; —C(═O)OR;
(8) —C(═O)NH₂; —C(═O)NHR; —C(═O)NR₂; —C(═O)NR^{N1}R^{N2};
(9) —NH₂; —NHR; —NR₂; —NR^{N1}R^{N2};
(10) —NHC(═O)H; —NRC(═O)H; —NHC(═O)R; —NRC(═O)R;
(11) —R;
(12) —R^{Alk}—OH; —R^{Alk}—OR
(13) —R^{Alk}NH₂; —R^{Alk}NHR; —R^{Alk}—NR; —R^{Alk}—NR^{N1}R^{N2};
(14) —R^{Alk}—C(═O)OH; —R^{Alk}—C(═O)OR;
(15) —R^{Alk}—C(═O)NH₂, —R^{Alk}—C(═O)NHR; —R^{Alk}—C(═O)NR₂; —R^{Alk}—C(═O)NR^{N1}R^{N2};
(17) -J-R^{Alk}—NH₂; -J-R^{Alk}—NHR; -J-R^{Alk}NR₂; -J-R^{Alk}NR^{N1}R^{N2};
(18) -J-R^{Alk}—C(═O)OH; -J-R^{Alk}—C(O)OR;
(19) -J-R^{Alk}—C(═O)NH₂; -J-R^{Alk}—C(═O)NHR;
(20) -J-R_{Alk}—C(═O)NR₂; -J-R^{Alk}—C(═O)NR^{N1}R^{N2};
wherein each J is independently selected from —O—, —NH—, or —NR—;
wherein each R is independently selected from:
(a) unsubstituted aliphatic C₁₋₆alkyl; substituted aliphatic C₁₋₆alkyl;
(b) unsubstituted aliphatic C₂₋₆alkenyl; substituted aliphatic C₂₋₆alkenyl;
(c) unsubstituted C₃₋₆cycloalkyl; substituted C₃₋₆cycloalkyl;
(d) unsubstituted C₆₋₁₀carboaryl; substituted C₆₋₁₀carboaryl;
(e) unsubstituted C₅₋₁₀heteroaryl; substituted C₅₋₁₀heteroaryl;
(f) unsubstituted C₆₋₁₀carboaryl-aliphatic C₁₋₄alkyl;
(g) substituted C₆₋₁₀carboaryl-aliphatic C₁₋₄alkyl;
(h) unsubstituted C₅₋₁₀heteroaryl-aliphatic C₁₋₄alkyl;
(i) substituted C₅₋₁₀heteroaryl-aliphatic C₁₋₄alkyl;
wherein each R^{Alk} is independently selected from:
(I) unsubstituted aliphatic C1-6alkylene; substituted aliphatic C1-6alkylene;
(II) unsubstituted aliphatic C2-6alkenylene; substituted aliphatic C2-6alkenylene;
(III) unsubstituted C3-6cycloalkylene; substituted C3-6cycloalkylene;
wherein in each group —NR^{N1}R^{N2}, independently, R^{N1} and R^{N2} taken together with the nitrogen atom to which they are attached form a ring having from 3 to 7 ring atoms:
and wherein R^{NA}, if present, is independently selected from:
(1) —H;
(11) —R;
(12) —R^{Alk}—OH; —R^{Alk}—OR
(13) —R^{Alk}—NH₂; —R^{Alk}—NHR; —R^{Alk}—NR₂; —R^{Alk}—NR^{N1}R^{N2};
(14) —R^{Alk}—C(═O)OH; —R^{Alk}—C(═O)OR;
(15) —R^{Alk}—C(═O)NH₂; R^{Alk}—C(═O)NHR; —R^{Alk}—C(═O)NR₂; —R^{Alk}—C(═O)NR^{N1}R^{N2};
wherein each R is independently selected from:
(a) unsubstituted aliphatic C₁₋₆alkyl; substituted aliphatic C₁₋₆alkyl;
(b) unsubstituted aliphatic C₂₋₆alkenyl; substituted aliphatic C₂₋₆alkenyl;
(c) unsubstituted C₃₋₆cycloalkyl; substituted C₃₋₆cycloalkyl;
(d) unsubstituted C₆₋₁₀carboaryl; substituted C₆₋₁₀carboaryl;
(e) unsubstituted C₅₋₁₀heteroaryl; substituted C₅₋₁₀heteroaryl;
(f) unsubstituted C₆₋₁₀carboaryl-aliphatic C₁₋₄alkyl;
(g) substituted C₆₋₁₀carboaryl-aliphatic C₁₋₄alkyl;
(h) unsubstituted C₆₋₁₀heteroaryl-aliphatic C₁₋₄alkyl;
(i) substituted C₅₋₁₀heteroaryl-aliphatic C₁₋₄alkyl;
wherein each R^{Alk} is independently selected from:
(I) unsubstituted aliphatic C₁₋₆alkylene; substituted aliphatic C₁₋₆alkylene;
(II) unsubstituted aliphatic C₂₋₆alkenylene; substituted aliphatic C₂₋₆alkenylene;
(III) unsubstituted C₃₋₆cycloalkylene; substituted C₃₋₆cycloalkylene;
wherein in each group —NR^{N1}R^{N2}, independently, R^{N1} and R^{N2} taken together with the nitrogen atom to which they are attached form a ring having from 3 to 7 ring atoms;
and wherein R^{NB} if present, is independently selected from:
(1) —H;
(11) —R;
(12) —R^{Alk}—OH; —R^{Alk}—OR
(13) —R^{Alk}—NH₂; —R^{Alk}—NHR; —R^{Alk}—NR₂; —R^{Alk}—NR^{N1}R^{N2};
(14) R^{Alk}—C(═O)OH; —R^{Alk}—C(═O)OR;
(15) —R^{Alk}—C(═O)NH₂; —R^{Alk}—C(═O)NHR; —R^{Alk}—C(═O)NR₂; —R^{Alk}—C(═O)NR^{N1}R^{N2};
wherein each R is independently selected from:
(a) unsubstituted aliphatic C₁₋₆alkyl; substituted aliphatic C₁₋₆alkyl;
(b) unsubstituted aliphatic C₂₋₆alkenyl; substituted aliphatic C₂₋₆alkenyl;
(c) unsubstituted C₃₋₆cycloalkyl; substituted C₃₋₆cycloalkyl;
(d) unsubstituted C₆₋₁₀carboaryl; substituted C₆₋₁₀carboaryl;
(e) unsubstituted C₅₋₁₀heteroaryl; substituted C₅₋₁₀heteroaryl;
(f) unsubstituted C₆₋₁₀carboaryl-aliphatic C₁₋₄alkyl;
(g) substituted C₆₋₁₀carboaryl-aliphatic C₁₋₄alkyl;
(h) unsubstituted C₅₋₁₀heteroaryl-aliphatic C₁₋₄alkyl;
wherein each R^{Alk} is independently selected from:
(I) unsubstituted aliphatic C₁₋₆alkylene; substituted aliphatic C₁₋₆alkylene;
(II) unsubstituted aliphatic C₂₋₆alkenylene; substituted aliphatic C₂₋₆alkenylene;
(III) unsubstituted C₃₋₆cycloalkylene; substituted C₃₋₆cycloalkylene;
wherein in each group —NR^{N1}R^{N2}, independently, R^{N1} and R^{N2} taken together with the nitrogen atom to which they are attached form a ring having from 3 to 7 ring atoms.

105-109. (canceled)

110. A method of treatment of a proliferative condition comprising administering to a patient in need of treatment a therapeutically effective amount a compound of the following formula: 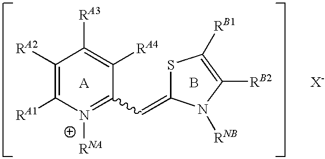
wherein:
R^{NA} and X⁻ may be present, or R^{NA} and X⁻ may be absent;
if R^{NA} and X⁻ are present, then:
the nitrogen atom of the A-ring bears a positive charge;
if R^{NA} and X⁻ are absent, then:
the nitrogen atom of the A-ring is electrically neutral;
and wherein:
(1-a) each of R^{A1}, R^{A2}, R^{A3}, and R^{A4} is independently an A-ring monovalent monodentate substituent; or:
(1-b) each of R^{A3} and R^{A4} is independently an A-ring monovalent monodentate substituent, and R^{A1} and R^{A2}, together with the carbon ring atoms of the A-ring to which they are attached, form a D-ring that is fused to the A-ring and which is:
(1-b)(i) a 6-membered carboaromatic ring, or
(1-b)(ii) a 5-membered heteroaromatic ring having exactly 1 heteroatom ring atom, which heteroatom is selected from N, O, and S, or
(1-b)(iii) a 5-membered heteroaromatic ring having exactly 2 heteroatom ring atoms, which heteroatoms are selected from N, O, and S;
and wherein the D-ring is unsubstituted, or is substituted with one or more D-ring substituents, provided that D-ring substituents do not form a further ring; or:
(1-c) each of R^{A1} and R^{A4} is independently an A-ring monovalent monodentate substituent, and R^{A2} and R^{A3}, together with the carbon ring atoms of the A-ring to which they are attached, form an E-ring that is fused to the A-ring and which is:
(1-c)(i) a 6-membered carboaromatic ring, or
(1-c)(ii) a 5-membered heteroaromatic ring having exactly 1 heteroatom ring atom, which heteroatom is selected from N, O, and S, or
(1-c)(iii) a 5-membered heteroaromatic ring having exactly 2 heteroatom ring atoms, which heteroatoms are selected from N, O, and S;
and wherein the E-ring is unsubstituted, or is substituted with one or more E-ring substituents, provided that E-ring substituents do not form a further ring; or:
(1-d) each of R^{A1} and R^{A2} is independently an A-ring monovalent monodentate substituent, and R^{A3} and R^{A4}, together with the carbon ring atoms of the A-ring to which they are attached, form a C-ring that is fused to the A-ring and which is:
(1-d)(i) a 6-membered carboaromatic ring, or
(1-d)(ii) a 5-membered heteroaromatic ring having exactly 1 heteroatom ring atom, which heteroatom is selected from N, O, and S, or
(1-d)(iii) a 5-membered heteroaromatic ring having exactly 2 heteroatom ring atoms, which heteroatoms are selected from N, O, and S;
and wherein the C-ring is unsubstituted, or is substituted with one or more C-ring substituents, provided that C-ring substituents do not form a further ring;
and wherein:
(2-a) each of R^{B1} and R^{B2} is independently a B-ring monovalent monodentate substituent; or:
(2-b) R^{B1} and R^{B2}, together with the carbon ring atoms of the B-ring to which they are attached, form an F-ring that is fused to the B-ring and which is:
(i) a 5-membered heteroaromatic ring having exactly 1 heteroatom ring atom, which heteroatom is selected from N, O, and S, or
(ii) a 5-membered heteroaromatic ring having exactly 2 heteroatom ring atoms, which heteroatoms are selected from N, O, and S;
and wherein the F-ring is unsubstituted, or is substituted with one or more F-ring substituents, provided that F-ring substituents do not form a further ring; or:
(2-c) R^{B1} and R^{B2}, together with the carbon ring atoms of the B-ring to which they are attached, form an F-ring that is fused to the B-ring and which is a 5-membered heteroaromatic ring having exactly 1 heteroatom ring atom, which heteroatom is selected from N, O, and S.
wherein the F-ring is substituted with two adjacent F-ring substituents which, together with the carbon ring atoms of the F-ring to which they are attached, form a G-ring that is fused to the F-ring and which is:
(2-c)(i) a 6-membered carboaromatic ring, or
(2-c)(ii) a 5-membered heteroaromatic ring having exactly 1 heteroatom ring atom, which heteroatom is selected from N, O, and S, or
(2-c)(iii) a 5-membered heteroaromatic ring having exactly 2 heteroatom ring atoms, which heteroatoms are selected from N, O, and S;
and wherein the G-ring is unsubstituted, or is substituted with one or more G-ring substituents, provided that G-ring substituents do not form a further ring;
and wherein:
R^{NA}, if present, is independently an A-ring nitrogen substituent;
R^{NB} is independently a B-ring nitrogen substituent;
the linkage joining the A-ring and the B-ring is cis or trans;
and pharmaceutically acceptable salts, solvates, amides, esters, ethers, N-oxides, chemically protected forms, and prodrugs thereof;
and wherein:
each D-ring substituent, if present, is independently a monovalent monodentate substituent or oxo;
each E-ring substituent, if present, is independently a monovalent monodentate substituent or oxo;
each C-ring substituent, if present, is independently a monovalent monodentate substituent or oxo;
each F-ring substituent, if present, is independently a monovalent monodentate substituent or oxo;
each G-ring substituent, if present, is independently a monovalent monodentate substituent or oxo;
and wherein each monovalent monodentate substituent is independently selected from:
(1) —H;
(2) —F; —Cl; —Br; —I;
(3) —OH; —OR;
(4) —SH; —SR;
(5) —NO₂;
(6) —C(═O)R;
(7) —C(═O)OH; —C(═O)OR;
(8) —C(═O)NH₂; —C(═O)NHR; —C(═O)NR₂; —C(═O)NR^{N1}R^{N2};
(9) —NH₂; —NHR; —NR₂; —NR^{N1}R^{N2};
(10) —NHC(═O)H; —NRC(═O)H; —NHC(═O)R; —NRC(═O)R;
(11) —R;
(12) —R^{Alk}—OH; —R^{Alk}—OR
(13) —R^{Alk}NH₂; —R^{Alk}NHR; —R^{Alk}—NR; —R^{Alk}—NR^{N1}R^{N2};
(14) —R^{Alk}—C(═O)OH; —R^{Alk}—C(═O)OR;
(15) —R^{Alk}—C(═O)NH₂, —R^{Alk}—C(═O)NHR; —R^{Alk}—C(═O)NR₂; —R^{Alk}—C(═O)NR^{N1}R^{N2};
(17) -J-R^{Alk}—NH₂; -J-R^{Alk}—NHR; -J-R^{Alk}NR₂; -J-R^{Alk}NR^{N1}R^{N2};
(18) -J-R^{Alk}—C(═O)OH; -J-R^{Alk}—C(O)OR;
(19) -J-R^{Alk}—C(═O)NH₂; -J-R^{Alk}—C(═O)NHR;
(20) -J-R_{Alk}—C(═O)NR₂; -J-R^{Alk}—C(═O)NR^{N1}R^{N2};
wherein each J is independently selected from —O—, —NH—, or —NR—;
wherein each R is independently selected from:
(a) unsubstituted aliphatic C₁₋₆alkyl; substituted aliphatic C₁₋₆alkyl;
(b) unsubstituted aliphatic C₂₋₆alkenyl; substituted aliphatic C₂₋₆alkenyl;
(c) unsubstituted C₃₋₆cycloalkyl; substituted C₃₋₆cycloalkyl;
(d) unsubstituted C₆₋₁₀carboaryl; substituted C₆₋₁₀carboaryl;
(e) unsubstituted C₅₋₁₀heteroaryl; substituted C₅₋₁₀heteroaryl;
(f) unsubstituted C₆₋₁₀carboaryl-aliphatic C₁₋₄alkyl;
(g) substituted C₆₋₁₀carboaryl-aliphatic C₁₋₄alkyl;
(h) unsubstituted C₅₋₁₀heteroaryl-aliphatic C₁₋₄alkyl;
(i) substituted C₅₋₁₀heteroaryl-aliphatic C₁₋₄alkyl;
wherein each R^{Alk} is independently selected from:
(I) unsubstituted aliphatic C1-6alkylene; substituted aliphatic C1-6alkylene;
(II) unsubstituted aliphatic C2-6alkenylene; substituted aliphatic C2-6alkenylene;
(III) unsubstituted C3-6cycloalkylene; substituted C3-6cycloalkylene;
wherein in each group —NR^{N1}R^{N2}, independently, R^{N1} and R^{N2} taken together with the nitrogen atom to which they are attached form a ring having from 3 to 7 ring atoms:
and wherein R^{NA}, if present, is independently selected from:
(1) —H;
(11) —R;
(12) —R^{Alk}—OH; —R^{Alk}—OR
(13) —R^{Alk}—NH₂; —R^{Alk}—NHR; —R^{Alk}—NR₂; —R^{Alk}—NR^{N1}R^{N2};
(14) —R^{Alk}—C(═O)OH; —R^{Alk}—C(═O)OR;
(15) —R^{Alk}—C(═O)NH₂; R^{Alk}—C(═O)NHR; —R^{Alk}—C(═O)NR₂; —R^{Alk}—C(═O)NR^{N1}R^{N2};
wherein each R is independently selected from:
(a) unsubstituted aliphatic C₁₋₆alkyl; substituted aliphatic C₁₋₆alkyl;
(b) unsubstituted aliphatic C₂₋₆alkenyl; substituted aliphatic C₂₋₆alkenyl;
(c) unsubstituted C₃₋₆cycloalkyl; substituted C₃₋₆cycloalkyl;
(d) unsubstituted C₆₋₁₀carboaryl; substituted C₆₋₁₀carboaryl;
(e) unsubstituted C₅₋₁₀heteroaryl; substituted C₅₋₁₀heteroaryl;
(f) unsubstituted C₆₋₁₀carboaryl-aliphatic C₁₋₄alkyl;
(g) substituted C₆₋₁₀carboaryl-aliphatic C₁₋₄alkyl;
(h) unsubstituted C₆₋₁₀heteroaryl-aliphatic C₁₋₄alkyl;
(i) substituted C₅₋₁₀heteroaryl-aliphatic C₁₋₄alkyl;
wherein each R^{Alk} is independently selected from:
(I) unsubstituted aliphatic C₁₋₆alkylene; substituted aliphatic C₁₋₆alkylene;
(II) unsubstituted aliphatic C₂₋₆alkenylene; substituted aliphatic C₂₋₆alkenylene;
(III) unsubstituted C₃₋₆cycloalkylene; substituted C₃₋₆cycloalkylene;
wherein in each group —NR^{N1}R^{N2}, independently, R^{N1} and R^{N2} taken together with the nitrogen atom to which they are attached form a ring having from 3 to 7 ring atoms;
and wherein R^{NB} if present, is independently selected from:
(1) —H;
(11) —R;
(12) —R^{Alk}—OH; —R^{Alk}—OR
(13) —R^{Alk}—NH₂; —R^{Alk}—NHR; —R^{Alk}—NR₂; —R^{Alk}—NR^{N1}R^{N2};
(14) R^{Alk}—C(═O)OH; —R^{Alk}—C(═O)OR;
(15) —R^{Alk}—C(═O)NH₂; —R^{Alk}—C(═O)NHR; —R^{Alk}—C(═O)NR₂; —R^{Alk}—C(═O)NR^{N1}R^{N2};
wherein each R is independently selected from:
(a) unsubstituted aliphatic C₁₋₆alkyl; substituted aliphatic C₁₋₆alkyl;
(b) unsubstituted aliphatic C₂₋₆alkenyl; substituted aliphatic C₂₋₆alkenyl;
(c) unsubstituted C₃₋₆cycloalkyl; substituted C₃₋₆cycloalkyl;
(d) unsubstituted C₆₋₁₀carboaryl; substituted C₆₋₁₀carboaryl;
(e) unsubstituted C₅₋₁₀heteroaryl; substituted C₅₋₁₀heteroaryl;
(f) unsubstituted C₆₋₁₀carboaryl-aliphatic C₁₋₄alkyl;
(g) substituted C₆₋₁₀carboaryl-aliphatic C₁₋₄alkyl;
(h) unsubstituted C₅₋₁₀heteroaryl-aliphatic C₁₋₄alkyl; (i) substituted C₅₋₁₀heteroaryl-aliphatic C₁₋₄alkyl;
wherein each R^{Alk} is independently selected from:
(I) unsubstituted aliphatic C₁₋₆alkylene; substituted aliphatic C₁₋₆alkylene;
(II) unsubstituted aliphatic C₂₋₆alkenylene; substituted aliphatic C₂₋₆alkenylene;
(III) unsubstituted C₃₋₆cycloalkylene; substituted C₃₋₆cycloalkylene;
wherein in each group —NR^{N1}R^{N2}, independently, R^{N1} and R^{N2} taken together with the nitrogen atom to which they are attached form a ring having from 3 to 7 ring atoms.

111. A method of treatment of cancer comprising administering to a patient in need of treatment a therapeutically effective amount a compound of the following formula: 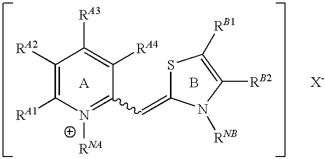
wherein:
R^{NA} and X⁻ may be present, or R^{NA} and X⁻ may be absent;
if R^{NA} and X⁻ are present, then:
the nitrogen atom of the A-ring bears a positive charge;
if R^{NA} and X⁻ are absent, then:
the nitrogen atom of the A-ring is electrically neutral;
and wherein:
(1-a) each of R^{A1}, R^{A2}, R^{A3}, and R^{A4} is independently an A-ring monovalent monodentate substituent; or:
(1-b) each of R^{A3} and R^{A4} is independently an A-ring monovalent monodentate substituent, and R^{A1} and R^{A2}, together with the carbon ring atoms of the A-ring to which they are attached, form a D-ring that is fused to the A-ring and which is:
(1-b)(i) a 6-membered carboaromatic ring, or
(1-b)(ii) a 5-membered heteroaromatic ring having exactly 1 heteroatom ring atom, which heteroatom is selected from N, O, and S, or
(1-b)(iii) a 5-membered heteroaromatic ring having exactly 2 heteroatom ring atoms, which heteroatoms are selected from N, O, and S;
and wherein the D-ring is unsubstituted, or is substituted with one or more D-ring substituents, provided that D-ring substituents do not form a further ring; or:
(1-c) each of R^{A1} and R^{A4} is independently an A-ring monovalent monodentate substituent, and R^{A2} and R^{A3}, together with the carbon ring atoms of the A-ring to which they are attached, form an E-ring that is fused to the A-ring and which is:
(1-c)(i) a 6-membered carboaromatic ring, or
(1-c)(ii) a 5-membered heteroaromatic ring having exactly 1 heteroatom ring atom, which heteroatom is selected from N, O, and S, or
(1-c)(iii) a 5-membered heteroaromatic ring having exactly 2 heteroatom ring atoms, which heteroatoms are selected from N, O, and S;
and wherein the E-ring is unsubstituted, or is substituted with one or more E-ring substituents, provided that E-ring substituents do not form a further ring; or:
(1-d) each of R^{A1} and R^{A2} is independently an A-ring monovalent monodentate substituent, and R^{A3} and R^{A4}, together with the carbon ring atoms of the A-ring to which they are attached, form a C-ring that is fused to the A-ring and which is:
(1-d)(i) a 6-membered carboaromatic ring, or
(1-d)(ii) a 5-membered heteroaromatic ring having exactly 1 heteroatom ring atom, which heteroatom is selected from N, O, and S, or
(1-d)(iii) a 5-membered heteroaromatic ring having exactly 2 heteroatom ring atoms, which heteroatoms are selected from N, O, and S;
and wherein the C-ring is unsubstituted, or is substituted with one or more C-ring substituents, provided that C-ring substituents do not form a further ring;
and wherein:
(2-a) each of R^{B1} and R^{B2} is independently a B-ring monovalent monodentate substituent; or:
(2-b) R^{B1} and R^{B2}, together with the carbon ring atoms of the B-ring to which they are attached, form an F-ring that is fused to the B-ring and which is:
(i) a 5-membered heteroaromatic ring having exactly 1 heteroatom ring atom, which heteroatom is selected from N, O, and S, or
(ii) a 5-membered heteroaromatic ring having exactly 2 heteroatom ring atoms, which heteroatoms are selected from N, O, and S;
and wherein the F-ring is unsubstituted, or is substituted with one or more F-ring substituents, provided that F-ring substituents do not form a further ring; or:
(2-c) RBI and R², together with the carbon ring atoms of the B-ring to which they are attached, form an F-ring that is fused to the B-ring and which is a 5-membered heteroaromatic ring having exactly 1 heteroatom ring atom, which heteroatom is selected from N, O, and S,
wherein the F-ring is substituted with two adjacent F-ring substituents which, together with the carbon ring atoms of the F-ring to which they are attached, form a G-ring that is fused to the F-ring and which is:
(2-c)(i) a 6-membered carboaromatic ring, or
(2-c)(ii) a 5-membered heteroaromatic ring having exactly 1 heteroatom ring atom, which heteroatom is selected from N, O, and S, or
(2-c)(iii) a 5-membered heteroaromatic ring having exactly 2 heteroatom ring atoms, which heteroatoms are selected from N, O, and S;
and wherein the G-ring is unsubstituted, or is substituted with one or more G-ring substituents, provided that G-ring substituents do not form a further ring;
and wherein:
R^{NA}, if present, is independently an A-ring nitrogen substituent;
R^{NB} is independently a B-ring nitrogen substituent;
the linkage joining the A-ring and the B-ring is cis or trans;
and pharmaceutically acceptable salts, solvates, amides, esters, ethers, N-oxides, chemically protected forms, and prodrugs thereof;
and wherein:
each D-ring substituent, if present, is independently a monovalent monodentate substituent or oxo;
each E-ring substituent, if present, is independently a monovalent monodentate substituent or oxo;
each C-ring substituent, if present, is independently a monovalent monodentate substituent or oxo;
each F-ring substituent, if present, is independently a monovalent monodentate substituent or oxo;
each G-ring substituent, if present, is independently a monovalent monodentate substituent or oxo;
and wherein each monovalent monodentate substituent is independently selected from:
(1) —H;
(2) —F; —Cl; —Br; —I;
(3) —OH; —OR;
(4) —SH; —SR;
(5) —NO₂;
(6) —C(═O)R;
(7) —C(═O)OH; —C(═O)OR;
(8) —C(═O)NH₂; —C(═O)NHR; —C(═O)NR₂; —C(═O)NR^{N1}R^{N2};
(9) —NH₂; —NHR; —NR₂; —NR^{N1}R^{N2};
(10) —NHC(═O)H; —NRC(═O)H; —NHC(═O)R; —NRC(═O)R;
(11) —R;
(12) —R^{Alk}—OH; —R^{Alk}—OR
(13) —R^{Alk}—NH₂; —R^{Alk}—NHR; —R^{Alk}—NR₂; —R^{Alk} NR^{N1}R^{N2};
(14) —R^{Alk}—C(═O)OH; —R^{Alk}—C(═O)OR;
(15) —R^{Alk}C(═O)NH₂; —R^{Alk}—C(═O)NHR; —R^{Alk}—C(═O)NR₂; —R^{Alk}—C(═O)NR^{N1}R^{N2};
(16) -J-R^{Alk}—OH; -J-R^{Alk}—OR
(17) -J-R^{Alk}—NH₂; -J-R^{Alk}—NHR; -J-R^{Alk}—NR₂; -J-R^{Alk}—NR^{N1}NR^{N2};
(18) -J-R^{Alk}—C(═O)OH; -J-R^{Alk}—C(═O)OR;
(19) -J-R^{Alk}—C(═O)NH₂; -J-R^{Alk}—C(═O)NHR;
(20) -J-R^{Alk}—C(═O)NR; -J-R^{Alk}—C(═O)NR^{N1}R^{N2};
wherein each J is independently selected from —O—, —NH—, or —NR—;
wherein each R is independently selected from:
(a) unsubstituted aliphatic C₁₋₆alkyl; substituted aliphatic C₁₋₆alkyl;
(b) unsubstituted aliphatic C₂₋₆alkenyl; substituted aliphatic C₂₋₆alkenyl;
(c) unsubstituted C₃₋₆cycloalkyl; substituted C₃₋₆cycloalkyl;
(d) unsubstituted C₆₋₁₀carboaryl; substituted C₆₋₁₀carboaryl;
(e) unsubstituted C₅₋₁₀heteroaryl; substituted C₅₋₁₀heteroaryl;
(f) unsubstituted C₆₋₁₀carboaryl-aliphatic C₁₋₄alkyl;
(g) substituted C₁₋₁₀carboaryl-aliphatic C₁₋₄alkyl;
(h) unsubstituted C₅₋₁₀heteroaryl-aliphatic C₁₋₄alkyl;
(i) substituted C₅₋₁₀heteroaryl-aliphatic C₁₋₄alkyl;
wherein each R^{Alk} is independently selected from:
(I) unsubstituted aliphatic C1-6alkylene; substituted aliphatic C1-6alkylene;
(II) unsubstituted aliphatic C2-6alkenylene; substituted aliphatic C2-6alkenylene;
(III) unsubstituted C3-6cycloalkylene; substituted C3-6cycloalkylene;
wherein in each group —NR^{N1}R^{N2}, independently, R^{N1} and R^{N2} taken together with the nitrogen atom to which they are attached form a ring having from 3 to 7 ring atoms;
and wherein R^{NA}, if present, is independently selected from:
(1) —H;
(11) —R;
(12) —R^{Alk}—OH; —R^{Alk}—OR
(13) —R^{Alk}—NH₂; —R^{Alk}—NHR; —R^{Alk}—NR₂; —R^{Alk}—NR^{N1}R^{N2};
(14) —R^{Alk}—C(═O)OH; —R^{Alk}—C(═O)OR;
(15) —R^{Alk}—C(═O)NH₂; R^{Alk}—C(═O)NHR; —R^{Alk}—C(═O)NR₂; —R^{Alk}—C(═O)NR^{N1}R^{N2};
wherein each R is independently selected from:
(a) unsubstituted aliphatic C₁₋₆alkyl; substituted aliphatic C₁₋₆alkyl;
(b) unsubstituted aliphatic C₂₋₆alkenyl; substituted aliphatic C₂₋₆alkenyl;
(c) unsubstituted C₃₋₆cycloalkyl; substituted C₃₋₆cycloalkyl;
(d) unsubstituted C₆₋₁₀carboaryl; substituted C₆₋₁₀carboaryl;
(e) unsubstituted C₅₋₁₀heteroaryl; substituted C₅₋₁₀heteroaryl;
(f) unsubstituted C₆₋₁₀carboaryl-aliphatic C₁₋₄alkyl;
(g) substituted C₆₋₁₀carboaryl-aliphatic C₁₋₄alkyl;
(h) unsubstituted C₆₋₁₀heteroaryl-aliphatic C₁₋₄alkyl;
(i) substituted C₅₋₁₀heteroaryl-aliphatic C₁₋₄alkyl;
wherein each R^{Alk} is independently selected from:
(I) unsubstituted aliphatic C₁₋₆alkylene; substituted aliphatic C₁₋₆alkylene;
(II) unsubstituted aliphatic C₂₋₆alkenylene; substituted aliphatic C₂₋₆alkenylene;
(III) unsubstituted C₃₋₆cycloalkylene; substituted C₃₋₆cycloalkylene;
wherein in each group —NR^{N1}R^{N2}, independently, R^{N1} and R^{N2} taken together with the nitrogen atom to which they are attached form a ring having from 3 to 7 ring atoms;
and wherein R^{NB} if present, is independently selected from:
(1) —H;
(11) —R;
(12) —R^{Alk}—OH; —R^{Alk}—OR
(13) —R^{Alk}—NH₂; —R^{Alk}—NHR; —R^{Alk}—NR₂; —R^{Alk}—NR^{N1}R^{N2};
(14) R^{Alk}—C(═O)OH; —R^{Alk}—C(═O)OR;
(15) —R^{Alk}—C(═O)NH₂; —R^{Alk}—C(═O)NHR; —R^{Alk}—C(═O)NR₂; —R^{Alk}—C(═O)NR^{N1}R^{N2};
wherein each R is independently selected from:
(a) unsubstituted aliphatic C₁₋₆alkyl; substituted aliphatic C₁₋₆alkyl;
(b) unsubstituted aliphatic C₂₋₆alkenyl; substituted aliphatic C₂₋₆alkenyl;
(c) unsubstituted C₃₋₆cycloalkyl; substituted C₃₋₆cycloalkyl;
(d) unsubstituted C₆₋₁₀carboaryl; substituted C₆₋₁₀carboaryl;
(e) unsubstituted C₅₋₁₀heteroaryl; substituted C₅₋₁₀heteroaryl;
(f) unsubstituted C₆₋₁₀carboaryl-aliphatic C₁₋₄alkyl;
(g) substituted C₆₋₁₀carboaryl-aliphatic C₁₋₄alkyl;
(h) unsubstituted C₅₋₁₀heteroaryl-aliphatic C₁₋₄alkyl; (i) substituted C₅₋₁₀heteroaryl-aliphatic C₁₋₄alkyl;
wherein each R^{Alk} is independently selected from:
(I) unsubstituted aliphatic C₁₋₆alkylene; substituted aliphatic C₁₋₆alkylene;
(II) unsubstituted aliphatic C₂₋₆alkenylene; substituted aliphatic C₂₋₆alkenylene;
(III) unsubstituted C₃₋₆cycloalkylene; substituted C₃₋₆cycloalkylene;
wherein in each group —NR^{N1}R^{N2}, independently, R^{N1} and R^{N2} taken together with the nitrogen atom to which they are attached form a ring having from 3 to 7 ring atoms.

112. A method according to claim 111, wherein the cancer is additionally characterised by one or more or all of the following:
(a) cells and/or tumours that have defective DNA mismatch repair (MMR);
(b) cells and/or tumours that have acquired defective DNA MMR following chemotherapy;
(c) cells and/or tumours that have microsatellite instability (MSI), and/or increased frameshift mutation frequency, and/or another measure of replication errors;
(d) the patient also having HNPCC syndrome;
(e) cells and/or tumours that have epigenetic and/or genetic changes/mutations/abnormalities at DNA MMR loci, such as hMSH2, hMSH6, hPMS2, hMSH3, hMLH1, hMLH3, and especially hMLH1;
(f) cells and/or tumours that acquire epigenetic and/or genetic changes/mutations/abnormalities at DNA MMR loci, such as hMSH2, hMSH6, hPMS2, hMSH3, hMLH1, hMLH3, and especially hMLH1, following chemotherapy;
(g) cells and/or tumours with reduced expression of DNA MMR genes and proteins;
(h) cells and/or tumours with defects in MMR signalling pathways, such as defects in p73, p53, JNK, ATM, CHK1, and CHK2 dependent cell cycle and apoptosis control, especially p53;
(i) cells and/or tumours that have acquired resistance to chemotherapy;
(j) cells and/or tumours that have relapsed following prior treatment, especially following treatment with cytotoxic chemotherapeutics, such as platinum based chemotherapeutics, such as platinum coordination complexes, such as cisplatin or carboplatin; monofunctional alkylating agents such as temodol/temozolomide; purine analogues such as 6-thioguanine; and topoisomerase II inhibitors such as doxorubicin;
(k) cells and/or tumours that have acquired resistance to treatment, especially following treatment with cytotoxic chemotherapeutics, such as platinum based chemotherapeutics, such as platinum coordination complexes, such as cisplatin or carboplatin; monofunctional alkylating agents such as temodol/temozolomide; purine analogues such as 6-thioguanine; and topoisomerase II inhibitors such as doxorubicin;
(l) cells and/or tumours that fail to respond to treatment with cytotoxic chemotherapeutics, such as platinum based chemotherapeutics, such as platinum coordination complexes, such as cisplatin or carboplatin; monofunctional alkylating agents such as temodol/temozolomide; purine analogues such as 6-thioguanine; and topoisomerase II inhibitors such as doxorubicin, after previously responding;
(m) cells and/or tumours that are therapy-related, such as therapy-related leukaemias arising following chemotherapy.

113. A method according to claim 111, wherein the cancer is additionally characterised by: loss of DNA mismatch repair (MMR).

114. A method according to claim 111, wherein the cancer is additionally characterised by proliferative cells characterised by loss of DNA mismatch repair (MMR).

115. A method according to claim 111, wherein the cancer is additionally characterised by acquisition of resistance to chemotherapy.

116. A method according to claim 111, wherein the cancer is additionally characterised by proliferative cells characterised by acquired cisplatin or carboplatin resistance.
